SEQUENCE LISTING (348 sequences)

SEQ ID NO: 1 - DNA, 65 bp, Artificial sequence
  Synthetic
cagctcactttcattttccgtaacactgagtttcaaaggaacgtccgtccgagatccgactacgc

SEQ ID NO: 2 - DNA, 65 bp, Artificial sequence
  Synthetic
gtcaacgcttagatttagcgccaaaaggaattaccaccacccgtgagctggagatccgactacgc

SEQ ID NO: 3 - DNA, 65 bp, Artificial sequence
  Synthetic
ggacggacttaactaaagatctccaaaaaaaaggcttttgcggtggacacgagatccgactacgc

SEQ ID NO: 4 - DNA, 65 bp, Artificial sequence
  Synthetic
gtgtccacttggatcgtcgggtagcaacggctacaagtacaaggtgccacgagatccgactacgc

SEQ ID NO: 5 - DNA, 65 bp, Artificial sequence
  Synthetic
gtggcaccttcggagattcgcgacctgctccatgacgtaacaggcaatgcgagatccgactacgc

SEQ ID NO: 6 - DNA, 65 bp, Artificial sequence
  Synthetic
gcattgccttaagctgctacaccagaacgagtagatcagttggcgttgacgagatccgactacgc

SEQ ID NO: 7 - DNA, 65 bp, Artificial sequence
  Synthetic
gcaactcgtttacaaactccgccaccctcagagcgaggcataccgtccacgagatccgactacgc

SEQ ID NO: 8 - DNA, 65 bp, Artificial sequence
  Synthetic
gtggacggttgtaagagcacaggtagaaagattctaaattgggcgaacacgagatccgactacgc

SEQ ID NO: 9 - DNA, 65 bp, Artificial sequence
  Synthetic
gctgcaccttggagccttagcggagtgagaataggtcaccagcgagttgcgagatccgactacgc

SEQ ID NO: 10 - DNA, 65 bp, Artificial sequence
  Synthetic
ggctcgactttgaggactagggagttaaaggccgctccaaaaggtgcagcgagatccgactacgc

SEQ ID NO: 11 - DNA, 65 bp, Artificial sequence
  Synthetic
gtgtcagcttccggaacgtaccaagcgcgaaacaagaggcttgtcgagccgagatccgactacgc

SEQ ID NO: 12 - DNA, 65 bp, Artificial sequence
  Synthetic
gtgttcgcttgcttgagattcattacccaaatcattacttaggctgacacgagatccgactacgc

SEQ ID NO: 13 - DNA, 65 bp, Artificial sequence
  Synthetic
cagccgagttcctcagaaacaacgcctgtagcataactttcaccacacctgagatccgactacgc

SEQ ID NO: 14 - DNA, 65 bp, Artificial sequence
  Synthetic
aggactgcttacattattaacactatcataacccaccgccacctcggctggagatccgactacgc

SEQ ID NO: 15 - DNA, 65 bp, Artificial sequence
  Synthetic
aggtgtggttacagtttctaattgtatcggtttaggtcgctggcgacatggagatccgactacgc

SEQ ID NO: 16 - DNA, 65 bp, Artificial sequence
  Synthetic
catgtcgcttaggcttgcaaagactttttcatgatgacccccgaacgatggagatccgactacgc

SEQ ID NO: 17 - DNA, 65 bp, Artificial sequence
  Synthetic
catcgttcttagcgattaaggcgcagacggtcaatgacaagagcctcacagagatccgactacgc

SEQ ID NO: 18 - DNA, 65 bp, Artificial sequence
  Synthetic
tgtgaggcttaccggatatggtttaatttcaactacggaacagcagtcctgagatccgactacgc

SEQ ID NO: 19 - DNA, 65 bp, Artificial sequence
  Synthetic
tgtcacggttcagccctctaccgccaccctcagatcgtttacaccgcatcgagatccgactacgc

SEQ ID NO: 20 - DNA, 65 bp, Artificial sequence
  Synthetic
gatgcggtttcagacgacttaataaaacgaactattaatcatgccagacagagatccgactacgc

SEQ ID NO: 21 - DNA, 65 bp, Artificial sequence
  Synthetic
cagcacagttctttcgagtgggattttgctaaactccacagaccgtgacagagatccgactacgc

SEQ ID NO: 22 - DNA, 65 bp, Artificial sequence
  Synthetic
acaccgagttccattaaacataaccgatatattctcagcttgctgtgctggagatccgactacgc

SEQ ID NO: 23 - DNA, 65 bp, Artificial sequence
  Synthetic
ggacaaggttgaaccgaactaaaacactcatcttggaagtttctcggtgtgagatccgactacgc

SEQ ID NO: 24 - DNA, 65 bp, Artificial sequence
  Synthetic
tgtctggctttgtgaatttcatcaagagtaatcttcataaggccttgtccgagatccgactacgc

SEQ ID NO: 25 - DNA, 65 bp, Artificial sequence
  Synthetic
gcaccgctttaggtttagatagttagcgtaacgaaaatgaatctgccaacgagatccgactacgc

SEQ ID NO: 26 - DNA, 65 bp, Artificial sequence
  Synthetic
gtcatggcttaatctacggataaaaaccaaaatatactcaggagcggtgcgagatccgactacgc

SEQ ID NO: 27 - DNA, 65 bp, Artificial sequence
  Synthetic
gttggcagtttttctgtagtgaatttcttaaacaacaaccatgctcagacgagatccgactacgc

SEQ ID NO: 28 - DNA, 65 bp, Artificial sequence
  Synthetic
gtctgagcttcgcccacgcgggtaaaatacgtaagaggcaaagtgctgtcgagatccgactacgc

SEQ ID NO: 29 - DNA, 65 bp, Artificial sequence
  Synthetic
gacagcacttagaatacactgaccaactttgaaaataggctgctgcatccgagatccgactacgc

SEQ ID NO: 30 - DNA, 65 bp, Artificial sequence
  Synthetic
ggatgcagttgctgacctaccttatgcgattttaggaagaaagccatgacgagatccgactacgc

SEQ ID NO: 31 - DNA, 65 bp, Artificial sequence
  Synthetic
ggcagacgttcttttgcaatcctgaatcttaccatataagtacggtcagcgagatccgactacgc

SEQ ID NO: 32 - DNA, 65 bp, Artificial sequence
  Synthetic
gctgaccgtttagcccggaataggtgtatcaccggcgagaggcgtctgccgagatccgactacgc

SEQ ID NO: 33 - DNA, 65 bp, Artificial sequence
  Synthetic
caggtggattgtaattgaaccagtcaggacgttgagaactggcgtcggtcgagatccgactacgc

SEQ ID NO: 34 - DNA, 65 bp, Artificial sequence
  Synthetic
gaccgacgttctcattatgcgctaatatcagagagtcagaggtccacctggagatccgactacgc

SEQ ID NO: 35 - DNA, 65 bp, Artificial sequence
  Synthetic
ggatgccattcttttgatctttccagacgttagttctaaagtcgtccagcgagatccgactacgc

SEQ ID NO: 36 - DNA, 65 bp, Artificial sequence
  Synthetic
gctggacgtttttgtcgtgatacaggagtgtactatacatggtggcatccgagatccgactacgc

SEQ ID NO: 37 - DNA, 65 bp, Artificial sequence
  Synthetic
caggacacttggaaccgctgcgccgacaatgacagcttgatacggagtacgagatccgactacgc

SEQ ID NO: 38 - DNA, 65 bp, Artificial sequence
  Synthetic
gtactccgttccgatagtctccctcagagccgccccaccaccgtgtcctggagatccgactacgc

SEQ ID NO: 39 - DNA, 65 bp, Artificial sequence
  Synthetic
gcctgcggtttagcaaactgtacagaccaggcgcgaggacagagtgcaacgagatccgactacgc

SEQ ID NO: 40 - DNA, 65 bp, Artificial sequence
  Synthetic
gttgcactttatgaacgggtagaaaatacatacacagtatgtccgcaggcgagatccgactacgc

SEQ ID NO: 41 - DNA, 65 bp, Artificial sequence
  Synthetic
gcctgacgttgaattagaccaacctaaaacgaaatgccactaccgtaagcgagatccgactacgc

SEQ ID NO: 42 - DNA, 65 bp, Artificial sequence
  Synthetic
gcttacggttcgaaggcagccagcaaaatcaccaccatttggcgtcaggcgagatccgactacgc

SEQ ID NO: 43 - DNA, 65 bp, Artificial sequence
  Synthetic
aggagcacttgttttaacccgtcgagagggttgaacgctaacagcgaaccgagatccgactacgc

SEQ ID NO: 44 - DNA, 65 bp, Artificial sequence
  Synthetic
ggttcgctttgagcgtctgaacaccctgaacaaagataacccgtttggacgagatccgactacgc

SEQ ID NO: 45 - DNA, 65 bp, Artificial sequence
  Synthetic
cagccgtgttaaccgccacgttccagtaagcgtcggtaataagtgctcctgagatccgactacgc

SEQ ID NO: 46 - DNA, 65 bp, Artificial sequence
  Synthetic
gtgatggcttattaccatatcaccggaaccagagaccctcagcacggctggagatccgactacgc

SEQ ID NO: 47 - DNA, 65 bp, Artificial sequence
  Synthetic
tggagtgcttgcaacataccgtcaccgacttgaggtagcaccgccatcacgagatccgactacgc

SEQ ID NO: 48 - DNA, 65 bp, Artificial sequence
  Synthetic
gtccaaacttacaagaataagactccttattacgtaaaggtggcactccagagatccgactacgc

SEQ ID NO: 49 - DNA, 65 bp, Artificial sequence
  Synthetic
gatggactttgataagtgggggtcagtgccttgacagtctctagcgtcctgagatccgactacgc

SEQ ID NO: 50 - DNA, 65 bp, Artificial sequence
  Synthetic
ggcaaccgttaattaactttccagagcctaatttaccaggcgagtccatcgagatccgactacgc

SEQ ID NO: 51 - DNA, 65 bp, Artificial sequence
  Synthetic
aggacgctttgaatttacccctcagagccaccactcttttcacggaggcagagatccgactacgc

SEQ ID NO: 52 - DNA, 65 bp, Artificial sequence
  Synthetic
tgcctccgtttaatcaaatagcaaggccggaaactaaaggtgcgagcacagagatccgactacgc

SEQ ID NO: 53 - DNA, 65 bp, Artificial sequence
  Synthetic
tgtgctcgttaattatcataaaagaaacgcaaagaagaactgctggtgccgagatccgactacgc

SEQ ID NO: 54 - DNA, 65 bp, Artificial sequence
  Synthetic
ggcaccagttgcatgatttgagttaagcccaatagacgggagcggttgccgagatccgactacgc

SEQ ID NO: 55 - DNA, 65 bp, Artificial sequence
  Synthetic
gatggtccttgcccgtatcggggttttgctcagtgccagttagcaggagcgagatccgactacgc

SEQ ID NO: 56 - DNA, 65 bp, Artificial sequence
  Synthetic
gctcctgcttcaaaataaacagggaagcgcattaataagagccggtcgcagagatccgactacgc

SEQ ID NO: 57 - DNA, 65 bp, Artificial sequence
  Synthetic
gaacagcgttccgccaccgccagaatggaaagcggtaacagtggaccatcgagatccgactacgc

SEQ ID NO: 58 - DNA, 65 bp, Artificial sequence
  Synthetic
gctaggcatttgaaaccattattagcgtttgccacctcagagcgctgttcgagatccgactacgc

SEQ ID NO: 59 - DNA, 65 bp, Artificial sequence
  Synthetic
gtgagccattgaataagtgacggaaattattcatgtcaccaatgcctagcgagatccgactacgc

SEQ ID NO: 60 - DNA, 65 bp, Artificial sequence
  Synthetic
tgcgaccgttaagaaacaataacggaatacccaaacaccacgtggctcacgagatccgactacgc

SEQ ID NO: 61 - DNA, 65 bp, Artificial sequence
  Synthetic
gcaagcggttaggattagaaacagttaatgccccataaatcctgccttacgagatccgactacgc

SEQ ID NO: 62 - DNA, 65 bp, Artificial sequence
  Synthetic
gaaggtcgttacataaaaacagccatattatttagaaggattccgcttgcgagatccgactacgc

SEQ ID NO: 63 - DNA, 65 bp, Artificial sequence
  Synthetic
gtaaggcatttcattaaaagaaccaccaccagagttcggtcacgctcatcgagatccgactacgc

SEQ ID NO: 64 - DNA, 65 bp, Artificial sequence
  Synthetic
gatgagcgtttagccccctcgatagcagcaccgtagggaaggcgacaccagagatccgactacgc

SEQ ID NO: 65 - DNA, 65 bp, Artificial sequence
  Synthetic
tggtgtcgtttaaatattttattttgtcacaatcccgaggaaccagtgcggagatccgactacgc

SEQ ID NO: 66 - DNA, 65 bp, Artificial sequence
  Synthetic
cgcactggttacgcaataatgaaatagcaatagcagagaatacgaccttcgagatccgactacgc

SEQ ID NO: 67 - DNA, 65 bp, Artificial sequence
  Synthetic
ggaccgtgttttcggaacctgagactcctcaagatcccaatccggacacagagatccgactacgc

SEQ ID NO: 68 - DNA, 65 bp, Artificial sequence
  Synthetic
tgtgtccgttcaaataagatagcagcctttacagtatcttaccctgttgcgagatccgactacgc

SEQ ID NO: 69 - DNA, 65 bp, Artificial sequence
  Synthetic
gtggctcgttagcattgatgatattcacaaacaactgcctatcacggtccgagatccgactacgc

SEQ ID NO: 70 - DNA, 65 bp, Artificial sequence
  Synthetic
catcgctcttgcgacagacgttttcatcggcattccgccgcccgagccacgagatccgactacgc

SEQ ID NO: 71 - DNA, 65 bp, Artificial sequence
  Synthetic
caggtgacttattcatatttcaaccgattgagggaatcagtagagcgatggagatccgactacgc

SEQ ID NO: 72 - DNA, 65 bp, Artificial sequence
  Synthetic
gcaacaggttcgaagcccagttaccagaaggaaaaatagaaagtcacctggagatccgactacgc

SEQ ID NO: 73 - DNA, 44 bp, Artificial sequence
  Synthetic
aaattcttaccagtataaagccaactttttggtgaggtggatag

SEQ ID NO: 74 - DNA, 44 bp, Artificial sequence
  Synthetic
gcctgtttagtatcatatgcgttattttttggtgaggtggatag

SEQ ID NO: 75 - DNA, 44 bp, Artificial sequence
  Synthetic
acaccggaatcataattactagaaatttttggtgaggtggatag

SEQ ID NO: 76 - DNA, 44 bp, Artificial sequence
  Synthetic
gataaataaggcgttaaataagaattttttggtgaggtggatag

SEQ ID NO: 77 - DNA, 44 bp, Artificial sequence
  Synthetic
tttaatggtttgaaataccgaccgttttttggtgaggtggatag

SEQ ID NO: 78 - DNA, 44 bp, Artificial sequence
  Synthetic
ttagttaatttcatcttctgacctatttttggtgaggtggatag

SEQ ID NO: 79 - DNA, 44 bp, Artificial sequence
  Synthetic
acgcgagaaaactttttcaaatatatttttggtgaggtggatag

SEQ ID NO: 80 - DNA, 44 bp, Artificial sequence
  Synthetic
gatgcaaatccaatcgcaagacaaatttttggtgaggtggatag

SEQ ID NO: 81 - DNA, 44 bp, Artificial sequence
  Synthetic
tgggttatataactatatgtaaatgtttttggtgaggtggatag

SEQ ID NO: 82 - DNA, 44 bp, Artificial sequence
  Synthetic
actacctttttaacctccggcttagtttttggtgaggtggatag

SEQ ID NO: 83 - DNA, 44 bp, Artificial sequence
  Synthetic
aatttatcaaaatcataggtctgagtttttggtgaggtggatag

SEQ ID NO: 84 - DNA, 44 bp, Artificial sequence
  Synthetic
ttaagacgctgagaagagtcaatagtttttggtgaggtggatag

SEQ ID NO: 85 - DNA, 44 bp, Artificial sequence
  Synthetic
tccttgaaaacatagcgatagcttatttttggtgaggtggatag

SEQ ID NO: 86 - DNA, 44 bp, Artificial sequence
  Synthetic
tcgctattaattaattttcccttagtttttggtgaggtggatag

SEQ ID NO: 87 - DNA, 44 bp, Artificial sequence
  Synthetic
agtgaataaccttgcttctgtaaattttttggtgaggtggatag

SEQ ID NO: 88 - DNA, 44 bp, Artificial sequence
  Synthetic
gaaacagtacataaatcaatatatgtttttggtgaggtggatag

SEQ ID NO: 89 - DNA, 44 bp, Artificial sequence
  Synthetic
atttcatttgaattaccttttttaatttttggtgaggtggatag

SEQ ID NO: 90 - DNA, 44 bp, Artificial sequence
  Synthetic
agaaaacaaaattaattacatttaatttttggtgaggtggatag

SEQ ID NO: 91 - DNA, 44 bp, Artificial sequence
  Synthetic
caaaagaagatgatgaaacaaacattttttggtgaggtggatag

SEQ ID NO: 92 - DNA, 44 bp, Artificial sequence
  Synthetic
gcgaattattcatttcaattacctgtttttggtgaggtggatag

SEQ ID NO: 93 - DNA, 44 bp, Artificial sequence
  Synthetic
aataccaagttacaaaatcgcgcagtttttggtgaggtggatag

SEQ ID NO: 94 - DNA, 44 bp, Artificial sequence
  Synthetic
caataacggattcgcctgattgctttttttggtgaggtggatag

SEQ ID NO: 95 - DNA, 44 bp, Artificial sequence
  Synthetic
taacagtaccttttacatcgggagatttttggtgaggtggatag

SEQ ID NO: 96 - DNA, 44 bp, Artificial sequence
  Synthetic
caggtttaacgtcagatgaatatactttttggtgaggtggatag

SEQ ID NO: 97 - DNA, 44 bp, Artificial sequence
  Synthetic
cagaaataaagaaattgcgtagatttttttggtgaggtggatag

SEQ ID NO: 98 - DNA, 44 bp, Artificial sequence
  Synthetic
ccatatcaaaattatttgcacgtaatttttggtgaggtggatag

SEQ ID NO: 99 - DNA, 44 bp, Artificial sequence
  Synthetic
tctgaataatggaagggttagaacctttttggtgaggtggatag

SEQ ID NO: 100 - DNA, 44 bp, Artificial sequence
  Synthetic
tataatcctgattgtttggattatatttttggtgaggtggatag

SEQ ID NO: 101 - DNA, 44 bp, Artificial sequence
  Synthetic
gattatcagatgatggcaattcatctttttggtgaggtggatag

SEQ ID NO: 102 - DNA, 44 bp, Artificial sequence
  Synthetic
aaggagcggaattatcatcatattctttttggtgaggtggatag

SEQ ID NO: 103 - DNA, 44 bp, Artificial sequence
  Synthetic
cattttgcggaacaaagaaaccacctttttggtgaggtggatag

SEQ ID NO: 104 - DNA, 44 bp, Artificial sequence
  Synthetic
taattttaaaagtttgagtaacatttttttggtgaggtggatag

SEQ ID NO: 105 - DNA, 44 bp, Artificial sequence
  Synthetic
gtattaaatcctttgcccgaacgtttttttggtgaggtggatag

SEQ ID NO: 106 - DNA, 44 bp, Artificial sequence
  Synthetic
tagactttacaaacaattcgacaactttttggtgaggtggatag

SEQ ID NO: 107 - DNA, 44 bp, Artificial sequence
  Synthetic
ataatacatttgaggatttagaagttttttggtgaggtggatag

SEQ ID NO: 108 - DNA, 44 bp, Artificial sequence
  Synthetic
caactaatagattagagccgtcaattttttggtgaggtggatag

SEQ ID NO: 109 - DNA, 44 bp, Artificial sequence
  Synthetic
tatctaaaatatctttaggagcacttttttggtgaggtggatag

SEQ ID NO: 110 - DNA, 44 bp, Artificial sequence
  Synthetic
actgatagccctaaaacatcgccattttttggtgaggtggatag

SEQ ID NO: 111 - DNA, 44 bp, Artificial sequence
  Synthetic
gaatggctattagtctttaatgcgctttttggtgaggtggatag

SEQ ID NO: 112 - DNA, 44 bp, Artificial sequence
  Synthetic
agaatacgtggcacagacaatattttttttggtgaggtggatag

SEQ ID NO: 113 - DNA, 44 bp, Artificial sequence
  Synthetic
atagaacccttctgacctgaaagcgtttttggtgaggtggatag

SEQ ID NO: 114 - DNA, 44 bp, Artificial sequence
  Synthetic
ataaaagggacattctggccaacagtttttggtgaggtggatag

SEQ ID NO: 115 - DNA, 44 bp, Artificial sequence
  Synthetic
gcagattcaccagtcacacgaccagtttttggtgaggtggatag

SEQ ID NO: 116 - DNA, 44 bp, Artificial sequence
  Synthetic
atcgtctgaaatggattatttacattttttggtgaggtggatag

SEQ ID NO: 117 - DNA, 44 bp, Artificial sequence
  Synthetic
atggaaatacctacattttgacgcttttttggtgaggtggatag

SEQ ID NO: 118 - DNA, 44 bp, Artificial sequence
  Synthetic
ccagccattgcaacaggaaaaacgctttttggtgaggtggatag

SEQ ID NO: 119 - DNA, 44 bp, Artificial sequence
  Synthetic
ctggtaatatccagaacaatattactttttggtgaggtggatag

SEQ ID NO: 120 - DNA, 44 bp, Artificial sequence
  Synthetic
gtagaagaactcaaactatcggccttttttggtgaggtggatag

SEQ ID NO: 121 - DNA, 44 bp, Artificial sequence
  Synthetic
tgattagtaataacatcacttgccttttttggtgaggtggatag

SEQ ID NO: 122 - DNA, 44 bp, Artificial sequence
  Synthetic
aaattaaccgttgtagcaatacttctttttggtgaggtggatag

SEQ ID NO: 123 - DNA, 44 bp, Artificial sequence
  Synthetic
ccgagtaaaagagtctgtccatcactttttggtgaggtggatag

SEQ ID NO: 124 - DNA, 44 bp, Artificial sequence
  Synthetic
gaagtgtttttataatcagtgaggctttttggtgaggtggatag

SEQ ID NO: 125 - DNA, 44 bp, Artificial sequence
  Synthetic
gacaggaacggtacgccagaatccttttttggtgaggtggatag

SEQ ID NO: 126 - DNA, 44 bp, Artificial sequence
  Synthetic
aacaggaggccgattaaagggattttttttggtgaggtggatag

SEQ ID NO: 127 - DNA, 44 bp, Artificial sequence
  Synthetic
tcctcgttagaatcagagcgggagctttttggtgaggtggatag

SEQ ID NO: 128 - DNA, 44 bp, Artificial sequence
  Synthetic
gctttgacgagcacgtataacgtgctttttggtgaggtggatag

SEQ ID NO: 129 - DNA, 44 bp, Artificial sequence
  Synthetic
cgccgctacagggcgcgtactatggtttttggtgaggtggatag

SEQ ID NO: 130 - DNA, 44 bp, Artificial sequence
  Synthetic
taaccaccacacccgccgcgcttaatttttggtgaggtggatag

SEQ ID NO: 131 - DNA, 44 bp, Artificial sequence
  Synthetic
tggcaagtgtagcggtcacgctgcgtttttggtgaggtggatag

SEQ ID NO: 132 - DNA, 44 bp, Artificial sequence
  Synthetic
aagcgaaaggagcgggcgctagggctttttggtgaggtggatag

SEQ ID NO: 133 - DNA, 44 bp, Artificial sequence
  Synthetic
cgaacgtggcgagaaaggaagggaatttttggtgaggtggatag

SEQ ID NO: 134 - DNA, 44 bp, Artificial sequence
  Synthetic
gatttagagcttgacggggaaagcctttttggtgaggtggatag

SEQ ID NO: 135 - DNA, 44 bp, Artificial sequence
  Synthetic
taaatcggaaccctaaagggagccctttttggtgaggtggatag

SEQ ID NO: 136 - DNA, 44 bp, Artificial sequence
  Synthetic
ttttggggtcgaggtgccgtaaagctttttggtgaggtggatag

SEQ ID NO: 137 - DNA, 44 bp, Artificial sequence
  Synthetic
tacgtgaaccatcacccaaatcaagtttttggtgaggtggatag

SEQ ID NO: 138 - DNA, 44 bp, Artificial sequence
  Synthetic
aaaccgtctatcagggcgatggccctttttggtgaggtggatag

SEQ ID NO: 139 - DNA, 44 bp, Artificial sequence
  Synthetic
acgtggactccaacgtcaaagggcgtttttggtgaggtggatag

SEQ ID NO: 140 - DNA, 44 bp, Artificial sequence
  Synthetic
tttggaacaagagtccactattaaatttttggtgaggtggatag

SEQ ID NO: 141 - DNA, 44 bp, Artificial sequence
  Synthetic
ccgagatagggttgagtgttgttcctttttggtgaggtggatag

SEQ ID NO: 142 - DNA, 44 bp, Artificial sequence
  Synthetic
aaatcccttataaatcaaaagaatatttttggtgaggtggatag

SEQ ID NO: 143 - DNA, 44 bp, Artificial sequence
  Synthetic
tgtttgatggtggttccgaaatcggtttttggtgaggtggatag

SEQ ID NO: 144 - DNA, 44 bp, Artificial sequence
  Synthetic
ctggtttgccccagcaggcgaaaattttttggtgaggtggatag

SEQ ID NO: 145 - DNA, 44 bp, Artificial sequence
  Synthetic
tgagagagttgcagcaagcggtccatttttggtgaggtggatag

SEQ ID NO: 146 - DNA, 44 bp, Artificial sequence
  Synthetic
agctgattgcccttcaccgcctggctttttggtgaggtggatag

SEQ ID NO: 147 - DNA, 44 bp, Artificial sequence
  Synthetic
tttcttttcaccagtgagacgggcatttttggtgaggtggatag

SEQ ID NO: 148 - DNA, 44 bp, Artificial sequence
  Synthetic
gtttgcgtattgggcgccagggtggtttttggtgaggtggatag

SEQ ID NO: 149 - DNA, 44 bp, Artificial sequence
  Synthetic
gaatcggccaacgcgcggggagaggtttttggtgaggtggatag

SEQ ID NO: 150 - DNA, 44 bp, Artificial sequence
  Synthetic
gaaacctgtcgtgccagctgcattatttttggtgaggtggatag

SEQ ID NO: 151 - DNA, 44 bp, Artificial sequence
  Synthetic
tgcgctcactgcccgctttccagtctttttggtgaggtggatag

SEQ ID NO: 152 - DNA, 44 bp, Artificial sequence
  Synthetic
gagtgagctaactcacattaattgctttttggtgaggtggatag

SEQ ID NO: 153 - DNA, 44 bp, Artificial sequence
  Synthetic
taaagtgtaaagcctggggtgcctatttttggtgaggtggatag

SEQ ID NO: 154 - DNA, 44 bp, Artificial sequence
  Synthetic
ttccacacaacatacgagccggaagtttttggtgaggtggatag

SEQ ID NO: 155 - DNA, 44 bp, Artificial sequence
  Synthetic
ctgtgtgaaattgttatccgctcactttttggtgaggtggatag

SEQ ID NO: 156 - DNA, 44 bp, Artificial sequence
  Synthetic
attcgtaatcatggtcatagctgtttttttggtgaggtggatag

SEQ ID NO: 157 - DNA, 44 bp, Artificial sequence
  Synthetic
tagaggatccccgggtaccgagctctttttggtgaggtggatag

SEQ ID NO: 158 - DNA, 44 bp, Artificial sequence
  Synthetic
caagcttgcatgcctgcaggtcgactttttggtgaggtggatag

SEQ ID NO: 159 - DNA, 44 bp, Artificial sequence
  Synthetic
acgacgttgtaaaacgacggccagttttttggtgaggtggatag

SEQ ID NO: 160 - DNA, 44 bp, Artificial sequence
  Synthetic
ttgggtaacgccagggttttcccagtttttggtgaggtggatag

SEQ ID NO: 161 - DNA, 44 bp, Artificial sequence
  Synthetic
agggggatgtgctgcaaggcgattatttttggtgaggtggatag

SEQ ID NO: 162 - DNA, 44 bp, Artificial sequence
  Synthetic
ctcttcgctattacgccagctggcgtttttggtgaggtggatag

SEQ ID NO: 163 - DNA, 44 bp, Artificial sequence
  Synthetic
ctgttgggaagggcgatcggtgcggtttttggtgaggtggatag

SEQ ID NO: 164 - DNA, 44 bp, Artificial sequence
  Synthetic
gcgccattcgccattcaggctgcgctttttggtgaggtggatag

SEQ ID NO: 165 - DNA, 44 bp, Artificial sequence
  Synthetic
cgcttctggtgccggaaaccaggcatttttggtgaggtggatag

SEQ ID NO: 166 - DNA, 44 bp, Artificial sequence
  Synthetic
atcgcactccagccagctttccggctttttggtgaggtggatag

SEQ ID NO: 167 - DNA, 44 bp, Artificial sequence
  Synthetic
gacgacgacagtatcggcctcaggatttttggtgaggtggatag

SEQ ID NO: 168 - DNA, 44 bp, Artificial sequence
  Synthetic
gtaaccgtgcatctgccagtttgagtttttggtgaggtggatag

SEQ ID NO: 169 - DNA, 44 bp, Artificial sequence
  Synthetic
ccacggtaggagtgcttgtcggacgagttgaaagtattaagagg

SEQ ID NO: 170 - DNA, 42 bp, Artificial sequence
  Synthetic
gacaagcactcctgtcctgatgcgactgagctggactcgtcc

SEQ ID NO: 171 - DNA, 44 bp, Artificial sequence
  Synthetic
ctattattctgaaacaccagctcagtcgcatcaggacctccatg

SEQ ID NO: 172 - DNA, 26 bp, Artificial sequence
  Synthetic
gtctggcatggagaccgtgggaggta

SEQ ID NO: 173 - DNA, 44 bp, Artificial sequence
  Synthetic
acaaggtcttcagcgtcatgctcgctacgtcagacgattggcct

SEQ ID NO: 174 - DNA, 42 bp, Artificial sequence
  Synthetic
catgacgctgaagcgaacggaagcacggttgcctgtagcgag

SEQ ID NO: 175 - DNA, 44 bp, Artificial sequence
  Synthetic
caggaggttgaggcagaggcaaccgtgcttccgttcgagtcggc

SEQ ID NO: 176 - DNA, 26 bp, Artificial sequence
  Synthetic
gtctgggccgactaccttgtgaggta

SEQ ID NO: 177 - DNA, 44 bp, Artificial sequence
  Synthetic
aaggctgtaggttcgtgctcagagacctgcgtcagactgtagcg

SEQ ID NO: 178 - DNA, 42 bp, Artificial sequence
  Synthetic
gagcacgaacctagtggtgatcgctcgtcgccagaggtctct

SEQ ID NO: 179 - DNA, 44 bp, Artificial sequence
  Synthetic
atcaagtttgcctttactggcgacgagcgatcaccacactccaa

SEQ ID NO: 180 - DNA, 26 bp, Artificial sequence
  Synthetic
gtctggttggagtcagccttgaggta

SEQ ID NO: 181 - DNA, 44 bp, Artificial sequence
  Synthetic
cagcatgaggtcacgtctacgtccactcagacaaaagggcgaca

SEQ ID NO: 182 - DNA, 42 bp, Artificial sequence
  Synthetic
gtagacgtgacctggttcgatgcgagacttcgtggagtggac

SEQ ID NO: 183 - DNA, 45 bp, Artificial sequence
  Synthetic
ggtttaccagcgccaacacgaagtctcgcatcgaaccgctatccg

SEQ ID NO: 184 - DNA, 26 bp, Artificial sequence
  Synthetic
gtctggggatagccatgctggaggta

SEQ ID NO: 185 - DNA, 44 bp, Artificial sequence
  Synthetic
cgtgcctgccataggacaccaccatgaggcagatagccgaacaa

SEQ ID NO: 186 - DNA, 42 bp, Artificial sequence
  Synthetic
ggtgtcctatggcgatggaagtcgaggtgactcgctcatggt

SEQ ID NO: 187 - DNA, 44 bp, Artificial sequence
  Synthetic
tttttaagaaaagtaacgagtcacctcgacttccatcacgcctt

SEQ ID NO: 188 - DNA, 26 bp, Artificial sequence
  Synthetic
gtctggaaggcgtaggcacggaggta

SEQ ID NO: 189 - DNA, 44 bp, Artificial sequence
  Synthetic
agcatcctcacactgcgagtagcaaggcaacgtcaaaaatgaaa

SEQ ID NO: 190 - DNA, 42 bp, Artificial sequence
  Synthetic
actcgcagtgtgaccgtacctctgtcctagtcgtgccttgct

SEQ ID NO: 191 - DNA, 44 bp, Artificial sequence
  Synthetic
aaacgattttttgtttacgactaggacagaggtacggatcagcc

SEQ ID NO: 192 - DNA, 26 bp, Artificial sequence
  Synthetic
gtctggggctgatggatgctgaggta

SEQ ID NO: 193 - DNA, 45 bp, Artificial sequence
  Synthetic
agggatagcaagcccacaacgtgaggacacttggaggctgcactc

SEQ ID NO: 194 - DNA, 42 bp, Artificial sequence
  Synthetic
tgtcctcacgttgctggatgccgatcctacgacacctccaag

SEQ ID NO: 195 - DNA, 45 bp, Artificial sequence
  Synthetic
cgctgacttgtcgtaggatcggcatccagataggaacccatgtac

SEQ ID NO: 196 - DNA, 28 bp, Artificial sequence
  Synthetic
ccagacgagtgcagagtcagcgtacctc

SEQ ID NO: 197 - DNA, 45 bp, Artificial sequence
  Synthetic
gaattgcgaataataagtgaccttgctgtaccgtcgagatggagt

SEQ ID NO: 198 - DNA, 42 bp, Artificial sequence
  Synthetic
acagcaaggtcaccgcagttggcactaggcgacatcgacggt

SEQ ID NO: 199 - DNA, 45 bp, Artificial sequence
  Synthetic
ccacaacctgtcgcctagtgccaactgcgttttttcacgttgaaa

SEQ ID NO: 200 - DNA, 28 bp, Artificial sequence
  Synthetic
ccagacactccatcggttgtggtacctc

SEQ ID NO: 201 - DNA, 45 bp, Artificial sequence
  Synthetic
accctcagcagcgaaacgagtacggcaacacggtgagagcctacg

SEQ ID NO: 202 - DNA, 42 bp, Artificial sequence
  Synthetic
gttgccgtactcgactggtcacgaacgtctccaactcaccgt

SEQ ID NO: 203 - DNA, 45 bp, Artificial sequence
  Synthetic
gctctgccttggagacgttcgtgaccagtgacagcatcggaacga

SEQ ID NO: 204 - DNA, 28 bp, Artificial sequence
  Synthetic
ccagaccgtaggctggcagagctacctc

SEQ ID NO: 205 - DNA, 45 bp, Artificial sequence
  Synthetic
tgtatcatcgcctgatcaacggtacgagatgcgaagcacagagtg

SEQ ID NO: 206 - DNA, 42 bp, Artificial sequence
  Synthetic
tctcgtaccgttgccagtagacctagccgacgtggcttcgca

SEQ ID NO: 207 - DNA, 45 bp, Artificial sequence
  Synthetic
gtcacgctcacgtcggctaggtctactggaaattgtgtcgaaatc

SEQ ID NO: 208 - DNA, 28 bp, Artificial sequence
  Synthetic
ccagaccactctgtagcgtgactacctc

SEQ ID NO: 209 - DNA, 45 bp, Artificial sequence
  Synthetic
cattcagtgaataaggacgctatgcctatcgctctaggacctctg

SEQ ID NO: 210 - DNA, 42 bp, Artificial sequence
  Synthetic
ataggcatagcgttgctccagtctgctgctcaggctagagcg

SEQ ID NO: 211 - DNA, 45 bp, Artificial sequence
  Synthetic
ccacgactcctgagcagcagactggagcacttgccctgacgagaa

SEQ ID NO: 212 - DNA, 28 bp, Artificial sequence
  Synthetic
ccagaccagaggtcagtcgtggtacctc

SEQ ID NO: 213 - DNA, 45 bp, Artificial sequence
  Synthetic
gaataccacattcaacaccgatgaggatcacggcactcgacactg

SEQ ID NO: 214 - DNA, 42 bp, Artificial sequence
  Synthetic
gatcctcatcggtcaagcgaaggtgcgagcctgtagtgccgt

SEQ ID NO: 215 - DNA, 45 bp, Artificial sequence
  Synthetic
gcggactgacaggctcgcaccttcgcttgtaatgcagatacataa

SEQ ID NO: 216 - DNA, 28 bp, Artificial sequence
  Synthetic
ccagaccagtgtcgcagtccgctacctc

SEQ ID NO: 217 - DNA, 45 bp, Artificial sequence
  Synthetic
cagccaagacgcaggtagcgagacagagctgaaagtattaagagg

SEQ ID NO: 218 - DNA, 42 bp, Artificial sequence
  Synthetic
tcgctacctgcgttcgtcggatggtgaggtccacgctctgtc

SEQ ID NO: 219 - DNA, 45 bp, Artificial sequence
  Synthetic
ctattattctgaaacagtggacctcaccatccgacgacacgagca

SEQ ID NO: 220 - DNA, 24 bp, Artificial sequence
  Synthetic
tggttgctcgtgcttggctggcat

SEQ ID NO: 221 - DNA, 50 bp, Artificial sequence
  Synthetic
cacggagtcgaagcgtaggacggtagccagtcagacgattggcctgtcct

SEQ ID NO: 222 - DNA, 37 bp, Artificial sequence
  Synthetic
acgcttcggaccttggtgatgctggactgtggctacc

SEQ ID NO: 223 - DNA, 45 bp, Artificial sequence
  Synthetic
caggaggttgaggcagcagtccagcatcaccaaggtcgctcggca

SEQ ID NO: 224 - DNA, 24 bp, Artificial sequence
  Synthetic
tctgtgccgagcactccgtgaggt

SEQ ID NO: 225 - DNA, 45 bp, Artificial sequence
  Synthetic
cagagccacggcatggtcttgcgttggaggcgtcagactgtagcg

SEQ ID NO: 226 - DNA, 42 bp, Artificial sequence
  Synthetic
caagaccatgccgacctcatcctcgctttcggtgctccaacg

SEQ ID NO: 227 - DNA, 45 bp, Artificial sequence
  Synthetic
atcaagtttgcctttacaccgaaagcgaggatgaggtgcggacga

SEQ ID NO: 228 - DNA, 24 bp, Artificial sequence
  Synthetic
tggttgctcgtgcttggctggcat

SEQ ID NO: 229 - DNA, 45 bp, Artificial sequence
  Synthetic
cacggagtctacggcagtgaccgatctccagacaaaagggcgaca

SEQ ID NO: 230 - DNA, 42 bp, Artificial sequence
  Synthetic
gtcactgccgtagctcacgaggcacaaccacagcggagatcg

SEQ ID NO: 231 - DNA, 45 bp, Artificial sequence
  Synthetic
ggtttaccagcgccaagctgtggttgtgcctcgtgaggctcggca

SEQ ID NO: 232 - DNA, 24 bp, Artificial sequence
  Synthetic
tctgtgccgagcactccgtgaggt

SEQ ID NO: 233 - DNA, 45 bp, Artificial sequence
  Synthetic
caaccgtcgttccacaggactcgcacttcgcagatagccgaacaa

SEQ ID NO: 234 - DNA, 42 bp, Artificial sequence
  Synthetic
agtcctgtggaacaccacgagacgccatcgagcggaagtgcg

SEQ ID NO: 235 - DNA, 45 bp, Artificial sequence
  Synthetic
tttttaagaaaagtaacgctcgatggcgtctcgtggtaagcctga

SEQ ID NO: 236 - DNA, 24 bp, Artificial sequence
  Synthetic
tggttgctcgtgcttggctggcat

SEQ ID NO: 237 - DNA, 45 bp, Artificial sequence
  Synthetic
cacggagtcaaggctacgagtcagacaggaacgtcaaaaatgaaa

SEQ ID NO: 238 - DNA, 42 bp, Artificial sequence
  Synthetic
actcgtagccttggaccgcactcaccactgctcgcctgtctg

SEQ ID NO: 239 - DNA, 45 bp, Artificial sequence
  Synthetic
aaacgattttttgtttcgagcagtggtgagtgcggtcgctcggca

SEQ ID NO: 240 - DNA, 46 bp, Artificial sequence
  Synthetic
agggatagcaagcccacaacgtgaggacacttggaggctgcactcg

SEQ ID NO: 241 - DNA, 42 bp, Artificial sequence; identical to SEQ ID NO: 194
  Synthetic

SEQ ID NO: 242 - DNA, 46 bp, Artificial sequence
  Synthetic
tcgctgacttgtcgtaggatcggcatccagataggaacccatgtac

SEQ ID NO: 243 - DNA, 26 bp, Artificial sequence
  Synthetic
cagacgagtgcagagtcagcgaatgc

SEQ ID NO: 244 - DNA, 46 bp, Artificial sequence
  Synthetic
gaattgcgaataataagtgaccttgctgtaccgtcgagatggagtc

SEQ ID NO: 245 - DNA, 42 bp, Artificial sequence; identical to SEQ ID NO: 198
  Synthetic

SEQ ID NO: 246 - DNA, 46 bp, Artificial sequence
  Synthetic
accacaacctgtcgcctagtgccaactgcgttttttcacgttgaaa

SEQ ID NO: 247 - DNA, 26 bp, Artificial sequence
  Synthetic
accagactccatcggttgtggtacct

SEQ ID NO: 248 - DNA, 46 bp, Artificial sequence
  Synthetic
accctcagcagcgaaacgagtacggcaacacggtgagagcctacgg

SEQ ID NO: 249 - DNA, 42 bp, Artificial sequence; identical to SEQ ID NO: 202
  Synthetic

SEQ ID NO: 250 - DNA, 46 bp, Artificial sequence
  Synthetic
tgctctgccttggagacgttcgtgaccagtgacagcatcggaacga

SEQ ID NO: 251 - DNA, 26 bp, Artificial sequence
  Synthetic
cagaccgtaggctggcagagcaatgc

SEQ ID NO: 252 - DNA, 46 bp, Artificial sequence
  Synthetic
tgtatcatcgcctgatcaacggtacgagatgcgaagcacagagtgc

SEQ ID NO: 253 - DNA, 42 bp, Artificial sequence; identical to SEQ ID NO: 206
  Synthetic

SEQ ID NO: 254 - DNA, 46 bp, Artificial sequence
  Synthetic
agtcacgctcacgtcggctaggtctactggaaattgtgtcgaaatc

SEQ ID NO: 255 - DNA, 26 bp, Artificial sequence
  Synthetic
accagcactctgtagcgtgactacct

SEQ ID NO: 256 - DNA, 46 bp, Artificial sequence
  Synthetic
cattcagtgaataaggacgctatgcctatcgctctaggacctctgg

SEQ ID NO: 257 - DNA, 42 bp, Artificial sequence; identical to SEQ ID NO: 210
  Synthetic

SEQ ID NO: 258 - DNA, 26 bp, Artificial sequence
  Synthetic
cagaccagaggtcagtcgtggaatgc

SEQ ID NO: 259 - DNA, 46 bp, Artificial sequence
  Synthetic
tccacgactcctgagcagcagactggagcacttgccctgacgagaa

SEQ ID NO: 260 - DNA, 46 bp, Artificial sequence
  Synthetic
gaataccacattcaacaccgatgaggatcacggcactcgacactgc

SEQ ID NO: 261 - DNA, 42 bp, Artificial sequence; identical to SEQ ID NO: 214
  Synthetic

SEQ ID NO: 262 - DNA, 46 bp, Artificial sequence
  Synthetic
agcggactgacaggctcgcaccttcgcttgtaatgcagatacataa

SEQ ID NO: 263 - DNA, 26 bp, Artificial sequence
  Synthetic
accagcagtgtcgcagtccgctacct

SEQ ID NO: 264 - DNA, 35 bp, Artificial sequence
  Synthetic
agggatagcaagcccattttcacatcgtcactcct

SEQ ID NO: 265 - DNA, 35 bp, Artificial sequence
  Synthetic
gaattgcgaataataattttcacatcgtcactcct

SEQ ID NO: 266 - DNA, 35 bp, Artificial sequence
  Synthetic
accctcagcagcgaaattttcacatcgtcactcct

SEQ ID NO: 267 - DNA, 35 bp, Artificial sequence
  Synthetic
tgtatcatcgcctgatttttcacatcgtcactcct

SEQ ID NO: 268 - DNA, 35 bp, Artificial sequence
  Synthetic
cattcagtgaataaggttttcacatcgtcactcct

SEQ ID NO: 269 - DNA, 35 bp, Artificial sequence
  Synthetic
gaataccacattcaacttttcacatcgtcactcct

SEQ ID NO: 270 - DNA, 35 bp, Artificial sequence
  Synthetic
ctattattctgaaacattttcacatcgtcactcct

SEQ ID NO: 271 - DNA, 35 bp, Artificial sequence
  Synthetic
caggaggttgaggcagttttcacatcgtcactcct

SEQ ID NO: 272 - DNA, 35 bp, Artificial sequence
  Synthetic
atcaagtttgcctttattttcacatcgtcactcct

SEQ ID NO: 273 - DNA, 35 bp, Artificial sequence
  Synthetic
ggtttaccagcgccaattttcacatcgtcactcct

SEQ ID NO: 274 - DNA, 35 bp, Artificial sequence
  Synthetic
tttttaagaaaagtaattttcacatcgtcactcct

SEQ ID NO: 275 - DNA, 35 bp, Artificial sequence
  Synthetic
aaacgattttttgtttttttcacatcgtcactcct

SEQ ID NO: 276 - DNA, 16 bp, Artificial sequence
  Synthetic
gaggagtgacgatgtg

SEQ ID NO: 277 - DNA, 20 bp, Artificial sequence
  Synthetic
tttttcacatcgtcactcct

SEQ ID NO: 278 - DNA, 59 bp, Artificial sequence
  Synthetic
ctattattctgaaacattttttttttttttttttttttttttttcacatcgtcactcct

SEQ ID NO: 279 - DNA, 59 bp, Artificial sequence
  Synthetic
caggaggttgaggcagttttttttttttttttttttttttttttcacatcgtcactcct

SEQ ID NO: 280 - DNA, 59 bp, Artificial sequence
  Synthetic
atcaagtttgcctttattttttttttttttttttttttttttttcacatcgtcactcct

SEQ ID NO: 281 - DNA, 59 bp, Artificial sequence
  Synthetic
ggtttaccagcgccaattttttttttttttttttttttttttttcacatcgtcactcct

SEQ ID NO: 282 - DNA, 59 bp, Artificial sequence
  Synthetic
tttttaagaaaagtaattttttttttttttttttttttttttttcacatcgtcactcct

SEQ ID NO: 283 - DNA, 59 bp, Artificial sequence
  Synthetic
aaacgattttttgtttttttttttttttttttttttttttttttcacatcgtcactcct

SEQ ID NO: 284 - DNA, 44 bp, Artificial sequence
  Synthetic
caactaatagattagagccgtcaatttttcacatcgtcactcct

SEQ ID NO: 285 - DNA, 44 bp, Artificial sequence
  Synthetic
tatctaaaatatctttaggagcactttttcacatcgtcactcct

SEQ ID NO: 286 - DNA, 44 bp, Artificial sequence
  Synthetic
actgatagccctaaaacatcgccatttttcacatcgtcactcct

SEQ ID NO: 287 - DNA, 44 bp, Artificial sequence
  Synthetic
gaatggctattagtctttaatgcgcttttcacatcgtcactcct

SEQ ID NO: 288 - DNA, 44 bp, Artificial sequence
  Synthetic
agaatacgtggcacagacaatatttttttcacatcgtcactcct

SEQ ID NO: 289 - DNA, 44 bp, Artificial sequence
  Synthetic
atagaacccttctgacctgaaagcgttttcacatcgtcactcct

SEQ ID NO: 290 - DNA, 44 bp, Artificial sequence
  Synthetic
ataaaagggacattctggccaacagttttcacatcgtcactcct

SEQ ID NO: 291 - DNA, 44 bp, Artificial sequence
  Synthetic
gcagattcaccagtcacacgaccagttttcacatcgtcactcct

SEQ ID NO: 292 - DNA, 44 bp, Artificial sequence
  Synthetic
atcgtctgaaatggattatttacatttttcacatcgtcactcct

SEQ ID NO: 293 - DNA, 44 bp, Artificial sequence
  Synthetic
atggaaatacctacattttgacgctttttcacatcgtcactcct

SEQ ID NO: 294 - DNA, 44 bp, Artificial sequence
  Synthetic
ccagccattgcaacaggaaaaacgcttttcacatcgtcactcct

SEQ ID NO: 295 - DNA, 44 bp, Artificial sequence
  Synthetic
ctggtaatatccagaacaatattacttttcacatcgtcactcct

SEQ ID NO: 296 - DNA, 44 bp, Artificial sequence
  Synthetic
gtagaagaactcaaactatcggcctttttcacatcgtcactcct

SEQ ID NO: 297 - DNA, 44 bp, Artificial sequence
  Synthetic
tgattagtaataacatcacttgcctttttcacatcgtcactcct

SEQ ID NO: 298 - DNA, 44 bp, Artificial sequence
  Synthetic
aaattaaccgttgtagcaatacttcttttcacatcgtcactcct

SEQ ID NO: 299 - DNA, 44 bp, Artificial sequence
  Synthetic
ccgagtaaaagagtctgtccatcacttttcacatcgtcactcct

SEQ ID NO: 300 - DNA, 44 bp, Artificial sequence
  Synthetic
gaagtgtttttataatcagtgaggcttttcacatcgtcactcct

SEQ ID NO: 301 - DNA, 44 bp, Artificial sequence
  Synthetic
gacaggaacggtacgccagaatcctttttcacatcgtcactcct

SEQ ID NO: 302 - DNA, 44 bp, Artificial sequence
  Synthetic
aacaggaggccgattaaagggatttttttcacatcgtcactcct

SEQ ID NO: 303 - DNA, 44 bp, Artificial sequence
  Synthetic
tcctcgttagaatcagagcgggagcttttcacatcgtcactcct

SEQ ID NO: 304 - DNA, 44 bp, Artificial sequence
  Synthetic
gctttgacgagcacgtataacgtgcttttcacatcgtcactcct

SEQ ID NO: 305 - DNA, 44 bp, Artificial sequence
  Synthetic
cgccgctacagggcgcgtactatggttttcacatcgtcactcct

SEQ ID NO: 306 - DNA, 44 bp, Artificial sequence
  Synthetic
taaccaccacacccgccgcgcttaattttcacatcgtcactcct

SEQ ID NO: 307 - DNA, 44 bp, Artificial sequence
  Synthetic
tggcaagtgtagcggtcacgctgcgttttcacatcgtcactcct

SEQ ID NO: 308 - DNA, 44 bp, Artificial sequence
  Synthetic
aagcgaaaggagcgggcgctagggcttttcacatcgtcactcct

SEQ ID NO: 309 - DNA, 44 bp, Artificial sequence
  Synthetic
cgaacgtggcgagaaaggaagggaattttcacatcgtcactcct

SEQ ID NO: 310 - DNA, 44 bp, Artificial sequence
  Synthetic
gatttagagcttgacggggaaagccttttcacatcgtcactcct

SEQ ID NO: 311 - DNA, 44 bp, Artificial sequence
  Synthetic
taaatcggaaccctaaagggagcccttttcacatcgtcactcct

SEQ ID NO: 312 - DNA, 44 bp, Artificial sequence
  Synthetic
ttttggggtcgaggtgccgtaaagcttttcacatcgtcactcct

SEQ ID NO: 313 - DNA, 44 bp, Artificial sequence
  Synthetic
tacgtgaaccatcacccaaatcaagttttcacatcgtcactcct

SEQ ID NO: 314 - DNA, 36 bp, Artificial sequence
  Synthetic
tcagtggacagccgttctggagcgttggacgaaact

SEQ ID NO: 315 - DNA, 28 bp, Artificial sequence
  Synthetic
ccagacagtttcgtggtcatcgtacctc

SEQ ID NO: 316 - DNA, 42 bp, Artificial sequence
  Synthetic
ccagaacggctgtggctaaacagtaaccgaagcaccaacgct

SEQ ID NO: 317 - DNA, 26 bp, Artificial sequence
  Synthetic
gtctggtagagcaccactgagaggta

SEQ ID NO: 318 - DNA, 36 bp, Artificial sequence
  Synthetic
cgatgacctgcttcggttactgtttagcctgctcta

SEQ ID NO: 319 - DNA, 47 bp, Artificial sequence
  Synthetic
ccagaacggctgtggctaaacagtaaccgaagcaccaacgctttttt

SEQ ID NO: 320 - DNA, 42 bp, Artificial sequence; identical to SEQ ID NO: 316
  Synthetic

SEQ ID NO: 321 - DNA, 38 bp, Artificial sequence
  Synthetic
cgtattggacatttccgtagaccgactggacatcttcg

SEQ ID NO: 322 - DNA, 26 bp, Artificial sequence
  Synthetic
cagacgaagatgtggtagtggaatgc

SEQ ID NO: 323 - DNA, 42 bp, Artificial sequence
  Synthetic
tctacggaaatgtggcagaatcaatcataagacaccagtcgg

SEQ ID NO: 324 - DNA, 24 bp, Artificial sequence
  Synthetic
tggtccttcacaccaatacggcat

SEQ ID NO: 325 - DNA, 38 bp, Artificial sequence
  Synthetic
tccactacctgtcttatgattgattctgcctgtgaagg

SEQ ID NO: 326 - DNA, 38 bp, Artificial sequence
  Synthetic
ctcagtggacagccgttctggagcgttggacgaaactc

SEQ ID NO: 327 - DNA, 26 bp, Artificial sequence
  Synthetic
accagagtttcgtggtcatcgtacct

SEQ ID NO: 328 - DNA, 42 bp, Artificial sequence; identical to SEQ ID NO: 316
  Synthetic

SEQ ID NO: 329 - DNA, 24 bp, Artificial sequence
  Synthetic
tctggtagagcaccactgagaggt

SEQ ID NO: 330 - DNA, 38 bp, Artificial sequence
  Synthetic
acgatgacctgcttcggttactgtttagcctgctctac

SEQ ID NO: 331 - DNA, 47 bp, Artificial sequence
  Synthetic
tctacggaaatgtggcagaatcaatcataagacaccagtcggttttt

SEQ ID NO: 332 - DNA, 42 bp, Artificial sequence; identical to SEQ ID NO: 323
  Synthetic

SEQ ID NO: 333 - DNA, 42 bp, Artificial sequence; identical to SEQ ID NO: 316
  Synthetic

SEQ ID NO: 334 - DNA, 42 bp, Artificial sequence; identical to SEQ ID NO: 323
  Synthetic

SEQ ID NO: 335 - DNA, 15 bp, Artificial sequence
  Synthetic
tccactacctgtctt

SEQ ID NO: 336 - DNA, 20 bp, Artificial sequence
  Synthetic
attctgcctgtgaaggacca

SEQ ID NO: 337 - DNA, 23 bp, Artificial sequence
  Synthetic
atgattgattctgcctgtgaagg

SEQ ID NO: 338 - DNA, 17 bp, Artificial sequence
  Synthetic
aagcgtagtcggatctc

SEQ ID NO: 339 - DNA, 17 bp, Artificial sequence
  Synthetic
ttctatccacctcacca

SEQ ID NO: 340 - DNA, 17 bp, Artificial sequence
  Synthetic
aactatccacctcacca

SEQ ID NO: 341 - DNA, 35 bp, Artificial sequence
  Synthetic
aggagtgacgatgtgttttataggaacccatgtac

SEQ ID NO: 342 - DNA, 35 bp, Artificial sequence
  Synthetic
aggagtgacgatgtgttttttttttcacgttgaaa

SEQ ID NO: 343 - DNA, 35 bp, Artificial sequence
  Synthetic
aggagtgacgatgtgttttgacagcatcggaacga

SEQ ID NO: 344 - DNA, 35 bp, Artificial sequence
  Synthetic
aggagtgacgatgtgttttaaattgtgtcgaaatc

SEQ ID NO: 345 - DNA, 35 bp, Artificial sequence
  Synthetic
aggagtgacgatgtgttttcttgccctgacgagaa

SEQ ID NO: 346 - DNA, 35 bp, Artificial sequence
  Synthetic
aggagtgacgatgtgtttttaatgcagatacataa

SEQ ID NO: 347 - DNA, 15 bp, Artificial sequence
  Synthetic
cacatcgtcactcct

SEQ ID NO: 348 - protein, 16 aa, Artificial sequence
  Synthetic
  MISC_FEATURE (1)..(1): Xaa=4-azido-L- phenylalanine
XGGAHIVMVDAYKPTK

(19) United States
(12) Patent Application Publication
(10) Pub. No.: US 2023/0304992 A1
(43) Pub. Date: Sep. 28, 2023

(54) GROWTH AND SITE-SPECIFIC ORGANIZATION OF MICRO-SCALE BIMOLECULAR DEVICES ON LIVING CELLS

(71) Applicant: The Johns Hopkins University, Baltimore, MD (US)

(72) Inventors: Rebecca B. Schulman, Baltimore, MD (US); Sisi Jia, Baltimore, MD (US); Siew Cheng Phua, Baltimore, MD (US); Yuta Nihongaki, Baltimore, MD (US); Yizeng Li, Baltimore, MD (US); Michael Pacella, Baltimore, MD (US); Yi Li, Baltimore, MD (US); Abdul Majeed Mohammed, Baltimore, MD (US); Sean X. Sun, Baltimore, MD (US); Takanari Inoue, Baltimore, MD (US)

(21) Appl. No.: 18/003,462

(22) PCT Filed: Jun. 29, 2021
(86) PCT No.: PCT/US2021/039526

§ 371 Date: Dec. 27, 2022

Related U.S. Application Data
(60) Provisional application No. 63/045,415, filed on Jun. 29, 2020.

(51) Int. Cl. G01N 33/50; C12N 5/00; C12N 15/11; B82Y 15/00

(52) U.S. Cl. CPC G01N 33/5005; C12N 5/0006; C12N 15/11; B82Y 15/00; C12N 2310/51; C12N 2310/351

(57) ABSTRACT

Described are conjugates of nucleic acid nanotubes and cells by linking the nanotubes to a cell surface moiety. Devices, kits, systems, and methods comprising the nanotube cell conjugates are also described.

### CROSS-REFERENCE TO RELATED APPLICATIONS

This application claims the benefit of U.S. Provisional Application No. 63/045,415, filed Jun. 29, 2020, the contents of which is herein incorporated by reference in its entirety.

### STATEMENT REGARDING FEDERAL FUNDING

This invention was made with government support under CMMI1562661, awarded by the National Science Foundation and D16AP00147, awarded by the Department of Defense/Defense Advanced Research Projects Agency. The government has certain rights in the invention.

### SEQUENCE LISTING

The text of the computer readable sequence listing filed herewith, titled “38440-601 SEQUENCE LISTING ST25”, created Jun. 28, 2021, having a file size of 69,884 bytes, is hereby incorporated by reference in its entirety.

### TECHNICAL FIELD

The present disclosure relates to conjugates of nucleic acid nanotubes and cells formed by linking the nanotubes or nanotube seeds to a cell surface moiety.

### BACKGROUND OF THE INVENTION

Cell function is determined by both types and concentrations of molecules comprising a cell and these molecules' spatial organization. Micron-scale assemblies, including membrane-bound and membrane-less organelles, cilia, cytoskeletal networks or the glycocalyx, provide specialized reaction environments, serve as transport conduits, and amplify chemical and mechanical signals in ways that individual molecules cannot. Understanding how to assemble synthetic micron-scale cell structures and to control their dynamics is a key goal of synthetic biology and nanotechnology in order to it possible to control such processes and create new function.

At the cell surface, controlling structure is important for controlling cell fate, drug and gene delivery, and for building biotic-abiotic interfaces. Nanoparticles, small molecules and nanowire cell-electronic interfaces have been attached to the cell surface. Controlling interactions between cell receptors and nanostructures has been studied in the context of therapeutic modulation of receptor activity, or for directing therapeutic import, less is known about creating and organizing microstructures that programmatically modify and extend cell surface architecture.

### SUMMARY OF THE INVENTION

The present invention is directed to a composition comprising: a cell and a nucleic acid nanotube, wherein a proximal end of the nanotube is conjugated using a linker to a moiety located on the surface of the cell. The cell-nanotube conjugates described herein allow functional integration of nucleic acid nanotubes into a cell's constantly changing architecture and overcome challenges of controlling the location and length of integration. In some embodiments, the nucleic acid nanotube is a DNA nanotube.

The present invention is also directed to methods for preparing a conjugate of a cell and a nucleic acid nanotube. In some embodiments, the methods comprise a) incubating a cell with a primary antibody configured to bind a moiety located on the surface of the cell; b) incubating the cell with bound primary antibody with a secondary antibody, wherein the secondary antibody comprises streptavidin; c) incubating a composition resulting from step b) with a biotinylated polynucleotide complementary to at least a portion of a single-stranded polynucleotide of the nanotube or at least a portion of a single-stranded polynucleotide of a nanotube seed; and d) incubating a composition resulting from step c) with a composition comprising the nanotube or a nanotube seed. In some embodiments, the methods comprise a) incubating a cell with a primary antibody configured to bind a moiety located on the surface of the cell; b) incubating the cell with bound primary antibody with a secondary antibody composition, wherein the second antibody is biotinylated; c) incubating a composition resulting from step b) with streptavidin or neutravidin; d) incubating a composition resulting from step c) with a biotinylated polynucleotide complementary to at least a portion of a single-stranded polynucleotide of the nanotube or at least a portion of a single-stranded polynucleotide of a nanotube seed; and e) incubating a composition resulting from step d) with a composition comprising the nanotube or a nanotube seed.

Also disclosed are methods, devices, and kits, comprising the compositions or cell-nucleic acid nanotube conjugates described herein.

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E are schematics of anchoring synthetic filaments, DNA nanotubes, to specific cell surface receptors. FIG. 1A shows DNA nanotubes anchored at specific locations on the cell surface could act as dynamic, functional elements of cells. FIG. 1B shows micron-scale DNA nanotubes self-assemble from monomer complexes. Arrows indicate 3′ ends of DNA strands. FIG. 1C shows polyethylene glycol (PEG, molecular weight 20 kD)-coated DNA nanotube monomers and assembled nanotubes. FIG. 1D shows nanotube seeds are scaffolded DNA origami structures that template nanotube growth. Seeds can be coated with DNA-PEG conjugates. FIG. 1E is a schematic of EGFR antibody-mediated, DNA-Directed Attachment (AMDA) for anchoring seeded DNA nanotubes to cell surface receptors to origami seeds on nanotube ends. Primary antibodies, biotinylated secondary antibodies, streptavidin or neutravidin, and biotinylated DNA form complex to present a DNA sequence. This sequence hybridizes to the complementary DNA sequence on a DNA nanotube seed.

FIGS. 2A-2I show PEG-coated DNA nanotube seeds attach to EGFR receptors via AMDA. Three-dimensional projection images of HeLa (FIG. 2A) and HEK293 (FIG. 2D) cells after AMDA or AMDA with BDC tag addition omitted to attach seeds to EGFR. Nanotube seeds were labeled with Atto488 (red) and secondary antibody-streptavidin conjugates with Alexa647 (blue). Scale bars: 20 μm. Confocal micrograph cross-sections of HeLa cells stained with DiD dye (blue) before seeds (red) were attached via AMDA (FIG. 2B). Scale bar: 20 μm. Average fluorescence intensities of nanotube seeds per HeLa cell after AMDA or after AMDA omitting different reagents (FIG. 2C). Confocal micrograph cross-sections of HEK293 cells (FIG. 2E). Scale bar: 20 μm. f HEK293 cell fluorescence in the channel used to label seeds after AMDA (orange), AMDA with BDC tag addition omitted (blue) and no AMDA (red), measured via flow cytometry. Average fluorescence intensities were 1423±75 (N=9818), 883±55 (N=9836) and 759±6 (N=9867). FIGS. 2G-2I show co-localization of nanotube seeds and antibodies on the cell membrane. Confocal micrograph cross-section of a HeLa cell after AMDA with secondary antibody labeled with Alexa647 (green) and nanotube seeds labeled with atto488 (red) (FIG. 2G). Scale bar: 10 μm. Computerized localization of antibodies (green) and seeds (red) of cell (FIG. 2H) from FIG. 2G. Fractions of nanotube seeds colocalized with antibody after AMDA and in randomized controls (FIG. 2H). Error bars here and elsewhere, unless otherwise stated, are 95% confidence intervals.

FIGS. 3A-3H Anchoring DNA nanotubes to EGFR receptors using AMDA. FIG. 3A is confocal micrographs of seeded nanotubes anchored to HeLa cells after EGFR AMDA and EGFR AMDA with BDC tag addition omitted. Seeds were labeled with atto647 (red) and nanotubes with Cy3 (green), streptavidin with Alexa488 (blue). Scale bar: 20 μm. FIG. 3B shows a three-dimensional reconstruction of a HeLa cell with seeded nanotubes attached to EGFR. Scale bar: 20 μm. FIG. 3C is a graph of the average fluorescence intensities of HeLa cells with seeded nanotube or seeds attached via AMDA and AMDA with BDC tag addition omitted. FIG. 3D is three-dimensional projection images of HEK293 cells with attached seeded nanotubes after AMDA and AMDA with BDC tag addition omitted. Scale bar: 20 μm. FIG. 3E is a three-dimensional reconstruction of an HEK293 cell with attached seeded nanotubes. Scale bar: 5 μm. FIG. 3F is a graph of average numbers of seeded nanotubes on HEK293 cells after AMDA and after AMDA with BDC tag addition omitted. Average numbers of attached seeds are shown in FIG. 2F FIG. 3G is confocal micrographs of HeLa cells at different times after seed attachment using EGFR AMDA (upper panel) and maximum projection images of HeLa cells at different times after seeded nanotube attachment using EGFR AMDA (lower panel). Scale bar: 20 μm. FIG. 3F is a graph of the fractions of DNA origami seeds or seeded nanotubes remaining on the cell surface at different times after AMDA (N=6 cells for each case). Fits are (a*e^{-bt}). For seeds, a=1.0±0.002, b=3.3±0.50 /h, for seeded nanotubes a=1.0±0.002, b=0.52±0.06 /h.

FIGS. 4A-4H show that anchored nanotubes indicate the magnitude of shear stress at the cell surface. FIG. 4A and FIG. 4B are side (FIG. 4A) and top (FIG. 4B) views of nanotube deflection in a flow Q in a rectangular channel. φ is the azimuthal angle between the plane of the nanotube and the x-axis. Φ is the total angle of nanotube rotation over a given time duration. FIG. 4C is a graph of the predicted distribution of φ by a simple model of deflection. FIG. 4D and FIG. 4E show confocal micrographs of seeded nanotubes anchored on the glass surface of a rectangular flow chamber (height=0.54mm, width=3.8mm) (FIG. 4D) and the top of HeLa cell membranes (FIG. 4E) in response to fluid shear stresses of 0, 0.05, 0.2, and 1 dyn/cm². Nanotubes were labeled with Cy3 (green), nanotube seeds with atto647 (red), and the cell membrane visualized with streptavidin-Alex488-conjugated EGFR antibodies (blue). Scale bars: 10 μm. FIG. 4F and FIG. 4G are maximum projection images of seeded nanotubes anchored on glass (FIG. 4F) and on HeLa cells (FIG. 4G) in response to fluid shear stresses 0, 0.1, 0.4, 1.2 and 1.6 dyn/cm². Scale bars: 2 μm. FIG. 4H is a graph of mean total angles of nanotubes as a function of fluid shear stress. N=15 nanotubes for each shear stress on both glass and cells.

FIGS. 5A-5E show nanotube growth on the surfaces of living cells. FIG. 5A is a schematic of nanotube growth via end-to-end joining and monomer addition. Seeds on anchored and capped nanotubes were unlabeled. Nanotubes were labeled with Cy3 (green), monomers with atto647 (red). FIG. 5B is two-color fluorescence micrographs of joined nanotubes anchored to live HeLa cells. Gentle fluid flow (shear stress 0.32 dyn/cm²) was applied to stretch the nanotubes for better characterization. Scale bar: 20 μm. Zoomed-in images of end-to-end joined nanotube structures on the cell surface. Scale bar: 5 μm. FIG. 5C is a graph of nanotube end-to-end joining yield quantification: Ent seeded nanotubes incubated with capped seeded nanotubes in solution at 37° C. for 3.5 h without additional monomers (FIG. 34), 4PEG seeded nanotubes anchored on glass surface incubated with capped nanotubes and additional 150 nM monomers added at 20° C. for 3.5 h (FIG. 36), 4PEG seeded nanotubes anchored on cell membrane incubated with capped nanotubes and additional 150 nM monomers at 20° C. for 4 h. FIGS. 5D and 5E show a schematic (FIG. 5D) and 3D projection images (FIG. 5E) of joined nanotubes (green and red) on a live HeLa cell after 0.6% methylcellulose (IMDM) was added. Cells were transfected with GFP (blue) to reveal cell shape and extent. Scale bar: 10 μm.

FIGS. 6A-D are schematics and structure of DNA during formation of DNA nanotubes.

FIGS. 7A and 7B show the architectures of Cy3 labeled 6nt nanotube monomers: SEs tiles with and without PEG. FIG. 7A shows a 6nt nanotube monomer without a PEG modification FIG. 7B shows a 6nt nanotube monomer with a PEG modification. The Ent SEs monomers were labeled with Cy3 by labeling the central strands SEs_3. Black triangles indicate the locations of crossover points.

FIG. 8 shows the architecture of atto647 labeled 6nt nanotube monomers. The Ent SEs monomers were labeled with atto647 by labeling the central strands SEs_3. Black triangles indicate the locations of crossover points.

FIGS. 9A and 9B show the architectures of Cy3 labeled 4nt monomers. FIG. 9A shows the SEd and REd tiles without PEG modification FIG. 9B shows the SEd and REd tile with PEG modification. The 4nt monomers were labeled with Cy3 by labeling both the central strands SEd_3 and REd_3. Black triangles indicate the locations of crossover points.

FIGS. 10A and 10B are schematic showing the architectures of the 4nt nanotube monomers labeled with atto488 (a) and atto647 (b) by labeling the central strands (REd_3 and SEd_3) with atto488 or atto647. Black triangles indicate the locations of crossover points.

FIGS. 11A and 11B show a 4nt inactive REd monomer activated by adding the “activation strand”, which replaced the strand HS_REd_4ntD1_3′ by strand displacement and exposed the REd monomer sticky ends to start assembling the nanotube. The inactive REd monomer could label with atto488 (FIG. 11A) or atto647 (FIG. 11B).

FIG. 12 is a scheme of DNA nanotube seeds to show the positions of adapters, PEG modification sites, fluorescent labeling sites, and the biotin attachments sites.

FIG. 13 is a map showing the positions of the staple strands, modification binding sites, and unused section of M13 DNA scaffold.

FIG. 14 is a map showing the positions of the staple strands, PEG-DNA conjugate binding sites, and fluorescence labeling strand sites and fluorescence labeling strands on the seed.

FIG. 15 shows the structure of a 15 nucleotide DNA overhang on a staple that serves as an attachment site for a PEG-DNA conjugate.

FIGS. 16A and 16B shows adapter organization on nanotube seeds. FIG. 16A is a map showing the positions of the seed A adapter and the corresponding left-side biotin attachment strands FIG. 16B is a map showing the positions of the seed B adapters and the corresponding right-side biotin attachment strands.

FIG. 17 shows the structures of the assembled seed A adapters for 6 nt nanotubes. The gray lines and corresponding sequences are parts of the M13mp18 scaffold.

FIG. 18 shows the structure of the assembled seed B adapters for 6 nt nanotubes. The gray lines and corresponding sequences are parts of the M13mp18 scaffold.

FIG. 19 shows the structures of the assembled seed A adapters for 4 nt nanotubes. The gray lines and corresponding sequences are parts of the M13mp18 scaffold.

FIG. 20 shows the structures of the assembled seed B adapters for 4 nt nanotubes. The gray lines and corresponding sequences are parts of the M13mp18 scaffold.

FIG. 21 is a schematic showing the positions of the BDC strand attachment sites on the PEG coated seeds. Six BDC attachment strands can be attached to the right barrel end of the seeds (opposite the end where nanotube growth occurs), and 30 BDC attachment strands can be attached at the center of the region of the M13 scaffold not used to form the seed structure.

FIGS. 22A and 22B show the structure of the BDC tag attachment sites at the left ends of nanotube seeds A (FIG. 16A) and right ends of nanotube seed B (FIG. 16B). The gray lines and corresponding sequences are parts of the M13mp18 scaffold. These attachment strands have one domain complementary to BDC tag and another domain complementary to the M13 scaffold at the left or right end of seeds.

FIG. 23 is a structure of the extended BDC tag attachment sites at the right ends of nanotube seeds. The gray lines and corresponding sequences are parts of the M13mp18 scaffold.

FIG. 24 shows the structure of the amino attachment sites at the left ends of nanotube seeds. The gray lines and corresponding sequences are parts of the M13mp18 scaffold. These attachment strands have one domain complementary to the SpyTag-DNA conjugate and another domain complementary to the M13 scaffold at the left end of seeds.

FIGS. 25A-25D shows that coating seeds with PEG reduces the extent of nonspecific binding between seeds and the HeLa cell membrane. FIG. 25A is 3D projection images of HeLa cells after being incubated with different concentrations (8, 16, 32, 64 pM) of nanotube seeds without PEG coating. Seeds were labeled with Atto488. The cells were located by their autofluorescence. FIG. 25B shows the average fluorescence intensity of seeds without PEG coating attached to a HeLa cell, as measured by mean fluorescence intensity per cell (N=15 cells per seed concentration). FIG. 25C is 3D projection images of HeLa cells after being incubated with different concentrations of nanotube seeds with PEG coating (8, 16, 32, 64 pM). FIG. 25D shows the average fluorescence intensity of seeds with PEG coating attached to a HeLa cell after incubation (N=15 cells per seed concentration). Scale bars: 20 μm.

FIGS. 26A-26D shows Epi-fluorescence micrographs and length distribution of seeded nanotube with 6 nucleotide sticky ends. Seeds were labeled with atto647 (red); monomers are labeled with Cy3 (green). Scale bars are 20 μm. FIGS. 26A-26B show epi-fluorescence micrograph (FIG. 26A) and histogram graph of length distribution (FIG. 26B) of nanotubes without PEG coating grown and incubated in 37° C. after adding nanotube seeds for 1 day. (N=139, three fields on view were measured). FIGS. 26C-26D show Epi-fluorescence micrograph (FIG. 26C) and histogram graph of length distribution (FIG. 26D) of nanotubes with PEG coating grown and incubated in 37° C. after adding nanotube seeds for 3 day. (N=396, ten fields on view were measured).

FIGS. 27A-27B show that PEG-coated nanotubes do not bind nonspecifically to the HeLa cell surface. The nanotubes were labeled with Cy3 (green). The cells were located by their autofluorescence. Scale bars 20 μm. FIG. 27A shows maximum intensity projection images of HeLa cells incubated with nanotubes grown from unmodified seeds (no PEG coating). Less than one seeded nanotube nonspecifically attached on each cell averagely (0.70±0.27 nanotube per cell (N=58)) FIG. 27B shows confocal stack micrograph of HeLa cells incubated with nanotubes grown from PEG-coated seeds. Less than one seeded nanotube nonspecifically attached on each cell on average (0.77±0.20 nanotube per cell (N=75))

FIGS. 28A-28C show anchoring SpyTag-conjugated, seeded nanotubes to the HeLa cells with SpyCatcher expressed on their membranes. Cells were stained as follows: Blue: cell with GFP, red: seeds labeled with atto647, green: nanotube labeled with Cy3. Scale bar: 20μm. FIG. 28A shows a scheme for conjugating a DNA strand to the SpyTag peptide. The azide modified Spytag peptide was conjugated with an amine-modified DNA strand that binds to the origami seed, Amine_DNA_SpyTag using a “copper-free click” reaction. These methods were adopted from Stephanopoulos et al. (Pflugers Archiv—European Journal of Physiology 454, 345-359, doi:10.1007/s00424-007-0212-8 (2007), incorporated herein by reference). The azide-SpyTag peptide was synthesized by BioSynthesis (Lot No. P3130-1) and its sequence is F^{az}-GGAHIVMVDAYKPTK (SEQ ID NO: 349), where Faz denotes the unnatural amino acid 4-azido-L-phenylalanine. The 3′ amine modified DNA strand was first reacted with dibenzocyclooctyne-sulfo-N-hydroxysuccinimidyl ester (DIBAC-sulfo-NHS) to generate the DNA-DIBAC conjugated. After 2 hours, the excess DIBAC-sulfo-NHS molecules were removed by a size exclusion spin column (Illustra Microspin G-25, GE Healthcare). The azido-Spytag and sodium chloride solution were added to the above purified DNA-DIBAC solution and then the reaction mixture was gently agitated overnight. FIG. 29B is a scheme for anchoring seeded nanotubes to the cell membrane through SpyCatcher-SpyTag interaction. The cell was transfected with a GFP-integrin-SpyCatcher plasmid designed to present the SpyCatcher protein outside the cell membrane. The SpyTag-DNA conjugate was attached to the ends of nanotube seed ends. FIG. 28C is confocal stack micrographs of HeLa cells after attaching the seeded nanotubes to the cell membrane. No significant difference in the number of nanotubes anchored on cells was observed between any of the control groups (SpyCatcher not present) and the experimental group.

FIG. 29 shows a schematic of the attachment of secondary antibody-modified seeds to HeLa cell membranes labeled with EGFR antibodies. Cells were labeled with the EGFR primary antibodies (1AB) and the nanotube seeds were modified by attaching secondary antibody-streptavidin (2AB-STA) molecules to biotin groups at the seeds' ends.

FIGS. 30A-30C show HeLa cells after attachment of PEG coated seeds to EGFR receptors via 6 BDC' strands on the seed barrel via AMDA. FIGS. 30A and 30B are confocal stack micrographs of HeLa cells after AMDA or AMDA without the addition of BDC tag as control using PEG coated seeds concentration 16 pM (FIG. 30A) and 64 pM (FIG. 30B). Seeds are labeled with Atto488 (red). Cells were located by autofluorescence and membrane locations are outlined. Scale bars 20 μm. FIG. 30C shows the quantification of the average fluorescence intensity per cell after either AMDA and after a control for AMDA processes in which 16 pM (left) or 64 pM (right) of seeds are incubated with cells. The average 488 nm fluorescence intensity of cells after AMDA using 16 pM of seeds was 2.2-fold greater than the average fluorescence intensity of cells after the AMDA control (P>0.05). The average fluorescence intensity of cells after AMDA using 64 pM of seeds was 7.4-fold greater than the average fluorescence intensity of cells after the AMDA control (P<0.05). Error bars are 95% confidence intervals.

FIG. 31 shows the number of DNA nanotube seeds attached to HeLa cells after AMDA with EGFR antibodies using different nanotube seed concentrations. The numbers of seeds are measured in terms of average measured fluorescence intensity per cell. Control experiments omitted the addition of one of the components needed for AMDA. Error bars are 95% confidence intervals.

FIGS. 32A-32C show the measurement of average fluorescent intensity using flow cytometry of suspended HEK293 cells with nanotube seeds attached using EGFR AMDA. The nanotube seeds were labeled with atto488. The HEK293 cells were otherwise unlabeled. Flow cytometry analysis of the blank sample (No AMDA step) (FIG. 32A), the control sample (No BDC tag) (FIG. 32B) and the experimental group sample (with all AMDA steps) (FIG. 32C). Based on cell size and granularity (forward and side scatter), the fluorescence signals of the cells inside the black circle were selected for analysis.

FIGS. 33A-33D show the attachment of nanotube seeds and seeded nanotubes to integrin receptors on HeLa cells using integrin AMDA. FIG. 33A is 3D projection images of HeLa cells with PEG-coated nanotube seeds attached to integrin receptors on HeLa cells using integrin AMDA. HeLa cells were transfected with GFP (blue) and the nanotube seeds were labeled with atto647 (red). FIG. 33B is the average fluorescence intensity in the seed channel per cell after integrin AMDA and after the control AMDA process where integrin antibodies were not added to cells. FIG. 33C is 3D projection images of HeLA cells with PEG-coated seeded nanotubes attached to integrin receptors on HeLa cells. FIG. 33D is the average fluorescence intensities in the nanotube seeds and seeded nanotube channels. Scale bars are 20 μm. Error bars are 95% confidence intervals.

FIGS. 34A and 34B shows measurement of the rate of end-to-end joining of Ent DNA nanotubes in solution. FIG. 34A shows the experimental design. Nanotubes are grown from seed A and from seed B in two batches and then combined to allow end-to-end joining. To make it possible to visualize the joining reaction, the nanotube monomers in the two batches are labelled with different colors and the seeds are also labeled with different colors. FIG. 34B is exemplary micrographs of nanotubes after 3.5 hours of incubation (upper panel) and 26 hours of incubation (lower panel). Seeds A labeled with atto488 (blue) and nanotubes grown from seeds A labeled with atto647 (red), seeds B labeled with atto647 (red) and nanotube grown from seeds B labeled with Cy3 (green). Scale bars 10 μm. 7±2% atto647 labeled nanotubes were joined by Cy3 labeled nanotubes after incubated at 37° C. for 3.5 h and this ratio increased to 30±6% after incubation for 26 h. 10±3% Cy3 labeled nanotubes were joined by atto647 labeled nanotubes after incubated at 37° C. for 3.5 h and this ratio increased to 30±6% after incubation for 26 h. Error bars are 95% confidence intervals.

FIG. 35 is a schematic for end-to-end joining of seeded nanotubes anchored to the cell membrane via the addition of DNA nanotubes.

FIGS. 36A and 36B show end-to-end joining of DNA nanotubes anchored to a glass surface to nanotubes in solution. FIG. 36A is a schematic of the process used to measure end-to-end joining of 4PEG nanotubes in the presence of extra monomers. FIG. 36B is confocal micrograph of nanotubes after joining under a gentle fluid flow (shear stress 0.32 dyn/cm²). 86±3% anchored nanotubes were extended by joined with the capped nanotubes with presents of 150 nM additional monomers after incubated at 20° C. for 4 hours. Scale bars of zoomed-in image: 2 μm.

FIGS. 37A and 37B are exemplary DNA sequences for nanotube seeds.

FIGS. 38A-38C are exemplary DNA sequences for fluorescence attachment strands.

FIGS. 39A and 39B are 6nt seed adapter exemplary DNA sequences.

FIGS. 40A and 40B are 4nt seed adapter exemplary DNA sequences.

FIGS. 41A-41E are biotin attachment strand exemplary DNA sequences.

FIGS. 42A-42C show specific attachment of seeded nanotubes on the cell membrane. FIG. 42A is a schematic showing that the A seeded nanotubes anchored to the cell membrane through simplified AMDA method. FIG. 42 B is fluorescence images of A seeded nanotubes anchored on the cell membrane. Nanotube seeds A labeled with Atto647 (red) and nanotube labeled with Cy3 (green). FIG. 42C is a graph of the quantification of the number of A seeded nanotube on cell membrane.

### DETAILED DESCRIPTION OF THE INVENTION

Disclosed herein is the organization of micron-scale filaments that can act as functional cellular elements on specific cell surface receptors. Micron-scale filaments are ubiquitous cell motifs that serve as sensors (antennae), mechanical supports, agents for generating motion, or for transport. Filaments must grow and be anchored in prescribed orientations to execute these functions.

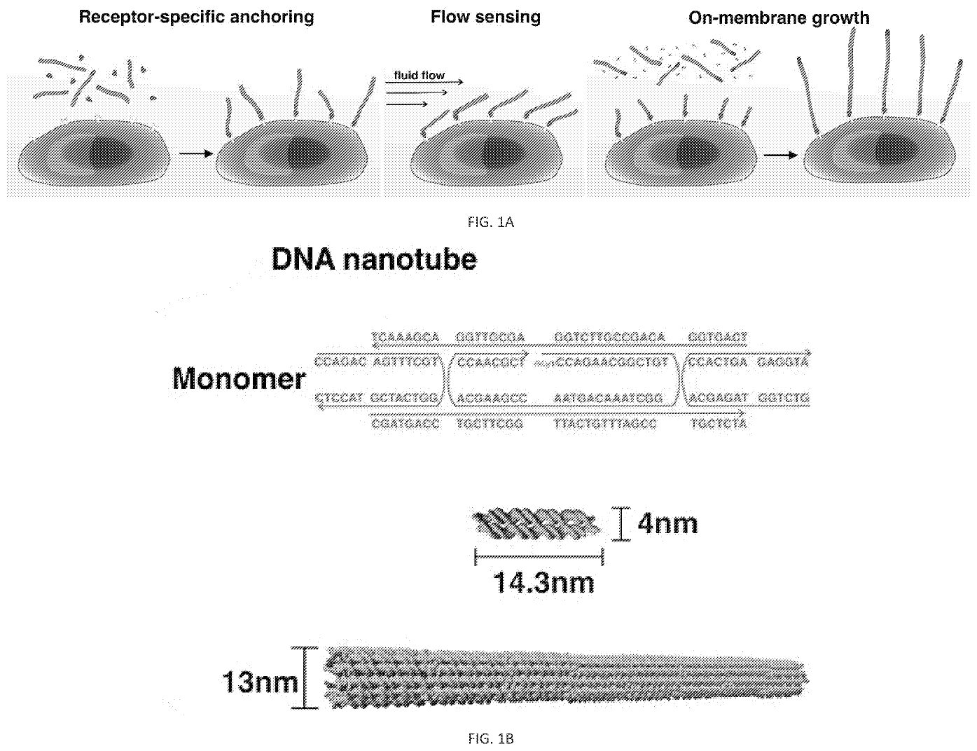

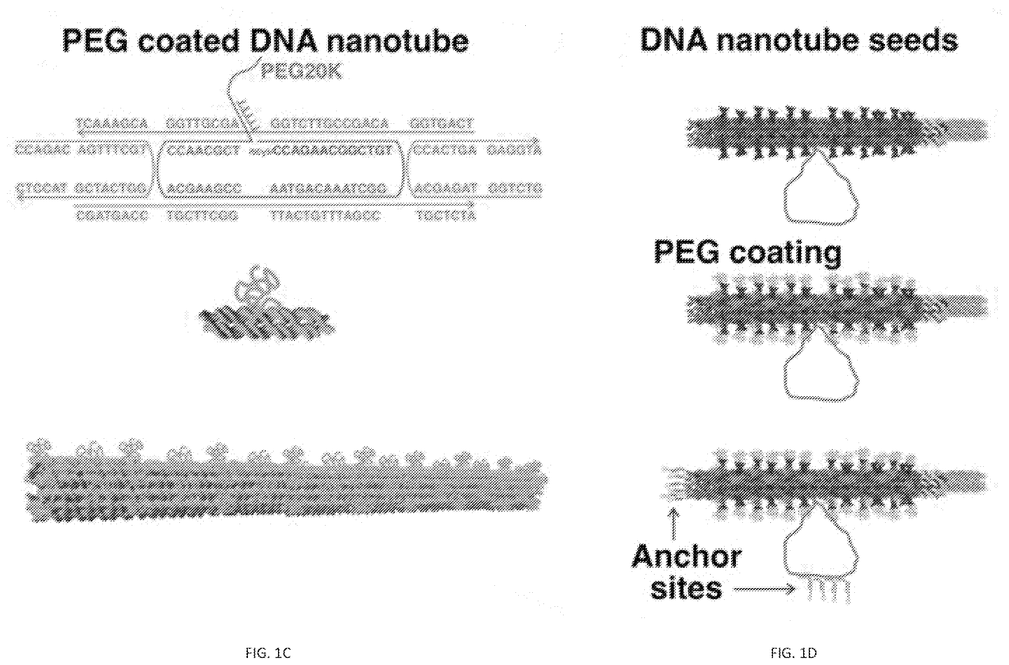

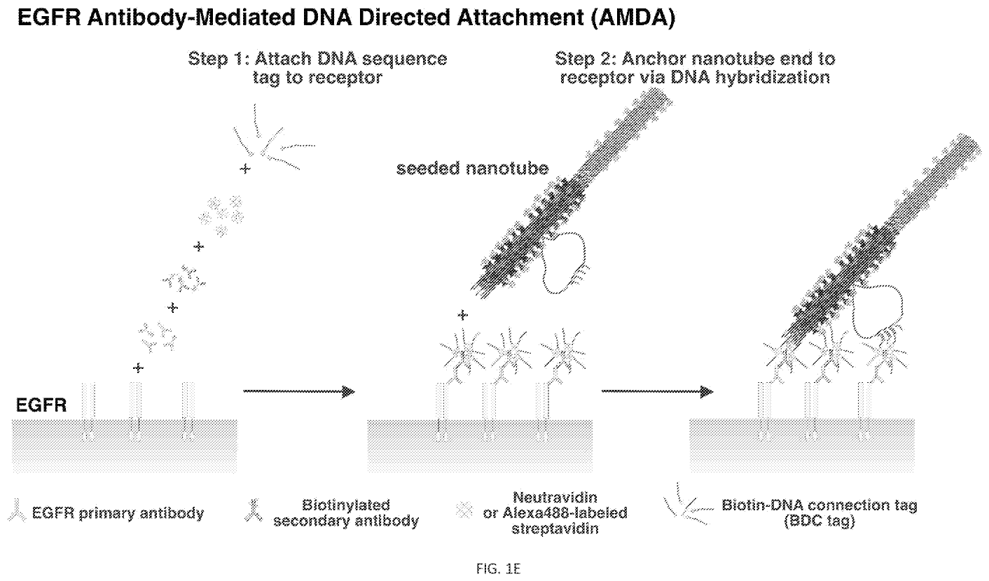

In some embodiments, the micron-scale filaments used herein are DNA tile nanotubes (FIG. 1B), semiflexible filaments with persistence length 8.7±0.5 μm (on order that of actin) that polymerize via Watson-Crick hybridization. These DNA nanotubes can grow from DNA origami templates, seeds (FIG. 1D), and can reach 100 μm. DNA nanotube growth kinetics, hierarchical assembly and diffusion rates have also been extensively characterized, providing a means to kinetically control their interactions with cells. Here, DNA nanotubes are anchored to specific receptors on the cell surface. The nanotubes can then be grown, demonstrating capacity for dynamic reorganization. The cell-anchored nanotubes can be used as sensitive flow rate meters whose dynamic range encompasses physiologically relevant rates of blood or ion channel-activated flow. These micron-scale structures can be attached to a cell at specific locations in specific orientations, extending the well-defined mesoscale architecture of the cell.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Cell,” as used herein, refers to the basic functional unit of life, and includes both prokaryotic and eukaryotic cells. Cells are characterized by an interior having the nucleus or nucleoid, and a cell membrane (cell surface). Cells can also have a cell wall. Cells without a cell wall include eukaryotic cells, mammalian cells, and stem cells. Cells with a cell wall include prokaryotic cells and plant cells. Other cells are useful in the present invention. In some embodiments, the cell is a mammalian cell.

“Complementarity” or “complementary to,” as used herein, refer to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary).

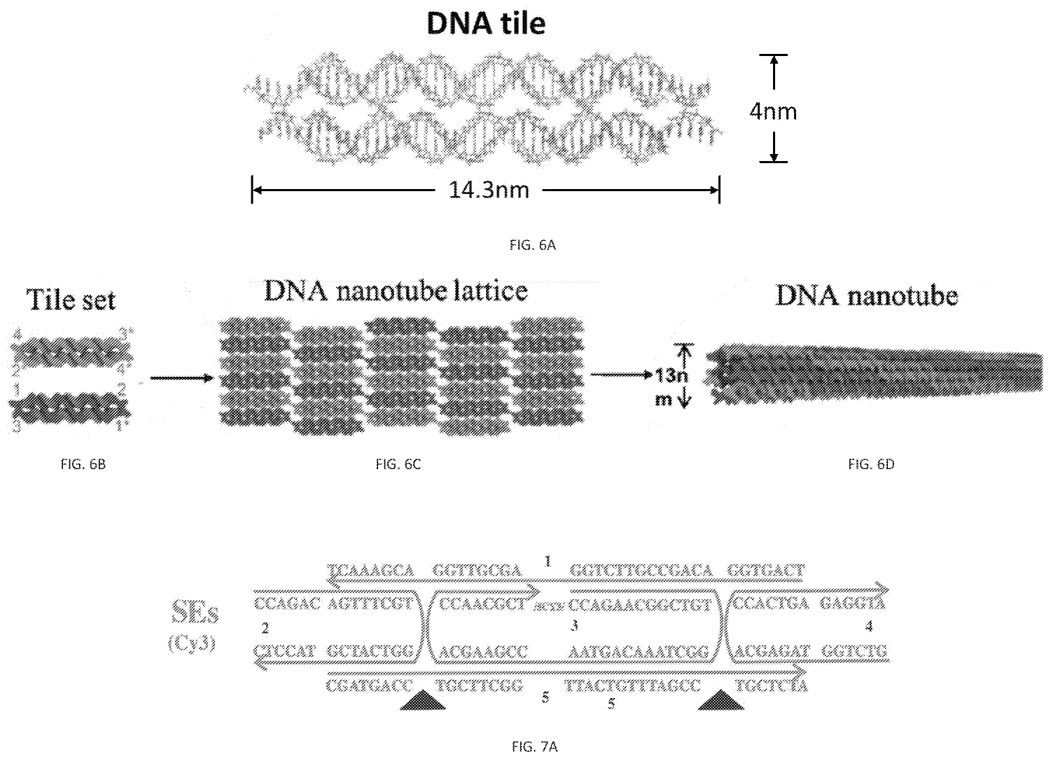

As used herein, “DNA nanotube” refers to a structure composed of individual units that form from five short DNA strands that self-assemble to form a rigid, brick-like structure, called a monomer (FIG. 6A) because of a preference for Watson-Crick complementarity. The sequence of these strands is random, non-genetic, and usually, made synthetically. Each of these basic units, or monomers has four locations where they join to similar sites on other monomers (FIG. 6B) in a pattern that allows them to assemble to form a repeating structure, or polymer (FIG. 6C & D). This growth process can happen at the same conditions that cells grow at, to allow nanotubes to grow concomitantly with the cells. The specificity of DNA hybridization and the predictable sequence-independent structure of the DNA double helix has enabled assembly of DNA nanotubes while controlling structure, circumference, and length, as well as functionalization to connections to a variety of other materials. See, for example, Rothemund, P. W. K., et al., J. Am. Chem. Soc. 2004, 126, 16344- 16352, Liu D. et al, Proc. Natl. Acad. Sci. U.S.A., 2004 101 (3) 717-722, Aldaye, F. A., et al., Nat. Nanotechnol. 2009, 4, 349-352, Mitchell, J. C., et al., J. Am. Chem. Soc. 2004, 126, 16342-16343, Wilner, O. I., et al., Nat. Commun. 2011, 2, 540, Liu H. et al, Angew Chem Int Ed Engl. 2006 Mar. 13; 45(12):1942-5, Yin, P., et al., Science 2008, 321, 824-826, Douglas, S. M., et al., Proc. Natl. Acad. Sci. U.S.A. 2007, 104, 6644-6648, Aldaye, F. A., et al., Science 2008, 321, 1795-1799, Bui, H., et al., Nano Lett. 2010, 10, 3367-3372, Sharma, J. et al., Science 2009, 323, 112-116, Shen, X., et al., J. Am. Chem. Soc. 2011, 134, 146-149, Mohammed, A. M. and Schulman, R. Nano Letters 13 (9) 4006-4013, 2013, Mohammed, A. M., et al., Nature Nanotechnology 12, 312-316, 2017, and Mohammed, A. M., et al., Nanoscale, 9, 522-526, 2017, all of which are incorporated herein by reference. Any of these methods, as well as those described elsewhere in the specification, may be used to form the DNA nanotubes used herein. In some embodiments, nanotubes can be assembled using the monomers formed by RNA strands or RNA/DNA strands. These monomers will assemble RNA nanotubes or RNA/DNA hybrid nanotubes, see, for example, Stewart J. M. et al, ACS Nano 2019, 13, 5, 5214-5221, Agarwal S. et al, J. Am. Chem. Soc. 2019, 141, 19, 7831-7841. In some embodiments, nanotubes can be assembled using process of nucleation from short nanotube segments, referred to as nanotube seeds. Nanotubes seeds can facilitate growth by the addition of DNA tile nanotubes or monomers. DNA nanotube monomers (also referred to as DNA tiles) hybridize to nanotube seeds and nanotubes via complementary single-stranded DNA ‘sticky ends,’ resulting in nanotube lengthening. See Mohammed, A. M., et al., Nature Nanotechnology 12, 312-316, 2017 and Mohammed, A. M., et al., Nanoscale, 9, 522-526, 2017, Jorgenson T. D., ACS Nano, 17, 1927-1936, 2017 incorporated herein by reference.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi stranded complex, a single self-hybridizing strand, or any combination of these. A second sequence that is complementary to a first sequence is referred to as the “complement” of the second sequence. The term “hybridizable” as applied to a polynucleotide refers to the ability of the polynucleotide to form a complex that is stabilized via hydrogen bonding between the bases of the nucleotide residues in a hybridization reaction.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide”, “protein,” and “peptide” are used interchangeably herein.

Any compositions or methods provided herein can be combined with one or more of any of the other compositions and methods provided herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### 2. CELL-NANOTUBE CONJUGATES

The present disclosure provides systems and compositions comprising cell-nucleic acid nanotube conjugates. In some embodiments, the nucleic acid nanotubes are DNA nanotubes, RNA nanotubes, or RNA/DNA hybrid nanotubes. The compositions and systems may comprise a cell and a nanotube in which the proximal end of the nanotube attached to a linker which is configured to bind a moiety located on the surface of the cell. The proximal end of the nanotube is configured (e.g., with a single-stranded polynucleotide or other end label) to allow connection to the linker. The distal end of the nanotube extends away from the cell.

In some embodiments, the nanotube may comprise a plurality of polyethylene glycol (PEG) molecules. The PEG molecule may have a molecular weight of 5-100 kDa. In some embodiments, the PEG molecule has a molecular weight of approximately 20 kDa. In some embodiments, the PEG molecule is a PEG-poly lysine copolymer.

The PEG molecules may be attached to the surface of the nanotube scaffold, effectively coating the nanotube surface. In some embodiments, the PEG molecules are covalently linked to a single-stranded polynucleotide which is complementary to a single-stranded polynucleotide exposed on the exterior surface of the nanotube. Thus, the hybridization of the complementary single-stranded polynucleotide results in the PEG conjugation to the nanotube.

The linker may include any configuration that connects the nanotube or nanotube seed to the cell, including for example at least one of antibody/antigen pairs, primary antibody/secondary antibody pairs, antibody-protein interactions (e.g., biotinylated secondary antibody-neutravidin or streptavidin), non-antibody protein-protein interactions, antibody-nucleotide biding, protein-nucleic acid binding, protein-small molecule binding, bioconjugation between reactive moieties (e.g., SpyTag/Spy Catcher), or any combination thereof. In some embodiments, the linker comprises a primary antibody configured to bind a moiety located on the surface of the cell. In some embodiments, the linker further comprises a corresponding secondary antibody. The secondary antibody binds to the primary antibody in a specific manner. In some embodiments, the secondary antibody is conjugated to streptavidin, or another multi-valent, high affinity biotin binding protein. In some embodiments, the secondary antibody is biotinylated and the linker further comprises streptavidin, or other multi-valent, high affinity biotin binding protein, e.g., neutravidin, bound to the biotinylated secondary antibody. In some embodiments, the primary antibody is conjugated to streptavidin, or another multi-valent, high affinity biotin binding protein.

The linker may further comprise biotinylated single-stranded polynucleotides which bind the streptavidin or the other multi-valent, high affinity biotin binding protein, either the individual molecule or that conjugated to the primary or secondary antibody. The biotinylated polynucleotide(s) may comprise any random nucleotide sequence that is complementary to at least a portion of a single-stranded polynucleotide of the DNA nanotube. The biotinylated single-stranded polynucleotides may comprise 10-50 nucleotides complementary to at least a portion of a single-stranded polynucleotide of the DNA nanotube. The single-stranded polynucleotides of the DNA nanotube may be located on the proximal end of the DNA nanotube. Alternatively, or additionally, the single-stranded polynucleotides may be exterior to the DNA nanotube, for example, the single-stranded polynucleotides may be connected by a long single-stranded DNA thread extending from the nanotube.

Essentially, the linker allows the nanotube to be conjugated to a moiety on the cell surface, including naturally occurring moieties, for example proteins, lipids, and carbohydrates. Alternatively, the cell may be engineered to comprise non-endogenous or modified cell surface moieties. In some embodiments, the moiety is a cell surface protein. In select embodiments, the moiety is a membrane receptor protein, a cell adhesion protein, or a transporter protein. The transporter protein may be a channel protein (e.g., ion channel proteins), a carrier protein (also called carriers, permeases, or transporters), or an ionophore.

The nanotube may further comprise an object linked to its distal end. Such that the nanotube is a mechanism to link a cell to another object. The object may include another cell, a drug or small molecule agent, or a surface of a device or container.

Any of the components, including the cell, nanotube, or the linker components may include a detectable label (e.g., fluorescent label, a radionuclide). In some embodiments, the nanotube may further comprise a detectable label. In some embodiments, the detectable label is outside the nanotube scaffold. In some embodiments, the detectable label is a fluorescent label.

### 3. USING CELL-NANOTUBE CONJUGATES

The present disclosure provides devices or substrate surfaces comprising the compositions or conjugates described herein. The cell may be in contact with the substrate surface, or alternatively, the cell may be suspended on the substrate surface through its connection with the nanotube which is tethered to the substrate surface.

The device or substrate surface can be of any suitable material. Examples of suitable materials include, but are not limited to, glass (including controlled-pore glass), polymers (e.g., polystyrene, polyurethane, polystyrene-divinylbenzene copolymer), silicone rubber, quartz, latex, a derivatizable transition metal, magnetic materials, silicon dioxide, silicon nitride, gallium arsenide, and derivatives thereof. The substrate surface can also have any suitable surface geometry, including, but not limited to, planar, curved and spherical. In some embodiments, the substrate surface is planar. In other embodiments, the substrate surface is spherical.

In some embodiments, the device also includes channels for fluid. In other embodiments, the channels are microchannels. In some other embodiments, the channels are nanochannels. In some embodiments, the device is adapted for biochemical analysis of the cell, including for example, fluorescence microscopy.

In another embodiment, the present invention provides a platform for use to observe and screen for processes such as wound healing, tissue regeneration, reactivity or cellular responsiveness to stimuli. Cells conjugated to nanotubes can be hybridized or annealed to each other in three dimensions, such as in a fluid or gel. Many applications of this technology are possible. For example, the cells can be used to study tissue regeneration. Any tissue can potentially be “grown” using a seeded cell attachment matrix to generate a network of interacting cells. The dynamics and products of such cell aggregates and simulated tissues can be studied and controlled. For example, stem cell differentiation and storage can be facilitated using the cell attachment system. For example, adult stem cells and induced pluripotent stem cells can be kept from differentiation or guided to a differentiated state as a desired cell type. Artificial tissues can be generated for replacing lost, damaged, or diseased tissue in animals including humans. Neural and spinal tissue regeneration can be controlled in the proper environment in vitro or in vivo.

In another embodiment, the cell-DNA nanotube conjugates can be used to monitor flow influences on cell. The conjugates may be used to measure or monitor shear stress, using the angles of DNA nanotubes in relation to flow and the cell surface. Methods of measuring shear stress may include obtaining a composition or cell-DNA nanotube conjugate as described herein; measuring an azimuth angle between the DNA nanotube and fluid flow direction with different fluid flow rates; and determining a mean total angle of DNA nanotube rotation for each fluid flow rate. Actuation of fluid flow may be implemented by external pressure sources, external mechanical pumps, or integrated mechanical micropumps in a microfluidic device.

The system or compositions can be used for delivery purpose, using the DNA nanotubes to deliver other agents, such as therapeutic agents or cells.

### 4. METHODS OF MAKING CELL-NANOTUBE CONJUGATES

The present disclosure also provides methods for preparing a conjugate of a cell and a nanotube as described herein.

In some embodiments, the methods comprise a) incubating a cell with a primary antibody configured to bind a moiety located on the surface of the cell; b) incubating the cell with bound primary antibody with a secondary antibody, wherein the secondary antibody comprises streptavidin or other multi-valent, high affinity biotin binding protein; c) incubating the composition resulting from step b) with a biotinylated polynucleotide complementary to at least a portion of a single-stranded polynucleotide of the nanotube or at least a portion of a single-stranded polynucleotide of a nanotube seed; and d) incubating the composition resulting from step c) with a composition comprising the nanotube or a nanotube seed.

In some embodiments, the methods comprise a) incubating a cell with a primary antibody configured to bind a moiety located on the surface of the cell; b) incubating the cell with bound primary antibody with a secondary antibody composition, wherein the second antibody is biotinylated; c) incubating the composition resulting from step b) with streptavidin or other multi-valent, high affinity biotin binding protein; d) incubating the composition resulting from step c) with a biotinylated polynucleotide complementary to at least a portion of a single-stranded polynucleotide of the nanotube or at least a portion of a single-stranded polynucleotide of a nanotube seed; and e) incubating the composition resulting from step d) with a composition comprising the nanotube or a nanotube seed.

Also contemplated herein are methods resulting from the combination of one or more of the disclosed steps into a single step.

In some embodiments, the methods comprise forming a secondary antibody conjugate, the method comprising incubating the second antibody, wherein the secondary antibody is biotinylated with streptavidin or other multi-valent, high affinity biotin binding protein, with a biotinylated polynucleotide complementary to at least a portion of a single-stranded polynucleotide of the nanotube and at least a portion of a single-stranded polynucleotide of a nanotube seed either sequentially or simultaneously. In some embodiments, the methods comprise: incubating a cell with a primary antibody configured to bind a moiety located on the surface of the cell; b) incubating the cell with bound primary antibody with the secondary antibody conjugate; c) incubating the composition resulting from step b) with a composition comprising the nanotube or a nanotube seed.

Alternatively, a primary antibody may be conjugated with polynucleotide complementary to at least a portion of a single-stranded polynucleotide of the nanotube or at least a portion of a single-stranded polynucleotide of a nanotube seed. Thus, in such instances, a primary antibody-based linker, without the use of a secondary antibody, is able to bind a moiety located on the surface of the cell and a nanotube or nanotube seed. The method may comprise conjugating a primary antibody with streptavidin to form a primary antibody-streptavidin conjugate and adding a biotinylated polynucleotide (also referred to herein as biotin-DNA connection or BDC) to the primary antibody-streptavidin conjugate to form the conjugate of primary antibody-streptavidin-biotinylated polynucleotide (1AB-DNA).

In some embodiments, the methods may further include incubating the resulting composition with additional nanotube seeds to add seeds to the distal end of the nanotube or initial nanotube seed.

After each step, the cells may be washed to remove any unbound component. For adhesive cells, the methods may be done while the cells are attached to the surface or to cells which have been trypsinized. For trypsinized adhesive cells or suspended cells, the methods may further comprise separating the cells from the unbound components using methods known in the art (e.g., centrifugation) and resuspension in fresh media or buffer. In some embodiments, the components for the next step may be added directly to the fresh media or buffer for use in the resuspension.

The descriptions of the nanotubes, nanotube seeds, primary antibody, secondary antibody, and biotinylated polynucleotide provided elsewhere in the disclosure are also relevant to the methods described herein.

### 5. KITS

Also within the scope of the present disclosure are kits that include the components of the present systems or compositions.

The kits may comprise a cell and a nanotube or nanotube seeds. The kit may further comprise at least one or all of: a primary antibody configured to bind a moiety located on the surface of the cell; a secondary antibody; a biotinylated single-stranded polynucleotide; and a plurality of nanotube monomers. The secondary antibody may be streptavidin, or other multi-valent, high affinity biotin binding protein, conjugated or biotinylated, which in this instance the kit further comprises streptavidin, or other multi-valent, high affinity biotin binding protein. The biotinylated single-stranded polynucleotide is complementary to at least a portion of a single-stranded polynucleotide of the DNA nanotube or DNA nanotube seeds. Any of the components in the kit may further comprise a fluorescent tag or label, or alternatively, the kit may further comprise a fluorescent tag or label configured to label any of the components of the kits (e.g., a cell stain or labeled single-stranded polynucleotide complementary to a single-stranded region of the nanotube. The descriptions of the nanotubes, nanotube seeds, primary antibody, secondary antibody, and biotinylated polynucleotide provided elsewhere in the disclosure are also relevant to the methods described herein.

Individual member components of the kits may be physically packaged together or separately. The components of the kit may be provided in bulk packages (e.g., multi-use packages) or single-use packages. The kits can also comprise instructions for using the components of the kit. The instructions are relevant materials or methodologies pertaining to the kit. The materials may include any combination of the following: background information, list of components and their availability information (purchase information, etc.), brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an interne website, or as recorded presentation.

It is understood that the disclosed kits can be employed in connection with the disclosed methods. The kit may further contain containers or devices for use with the methods, compositions or systems disclosed herein. The kits optionally may provide additional components such as buffers and disposable single-use equipment (e.g., pipettes, cell culture plates or flasks).

The kits provided herein are in suitable packaging. Suitable packaging includes, but is not limited to, vials, bottles, jars, flexible packaging, and the like.

### 6. EXAMPLES

### Materials and Methods

Reagents. M13mp18 scaffold strand was purchased from Bayou Biolabs. All other DNA strands used in this study were synthesized by Integrated DNA Technologies, Inc. (IDT). Strands for the DNA nanotube tiles and the adapter strands for the DNA nanotube seeds, Cy3-, ATT0647- and ATT0488-, biotin-labeled strands and amino-modified strands were HPLC purified. All other strands were simply desalted. Concentrations of DNA strands were determined either by measuring absorbance at 260 nm (using extinction coefficients supplied by IDT) or by relying on IDT to determine solution concentrations. N-hydroxylsuccinimide (NETS) functionalized polyethylene glycol (molecular weight 20K) (PEG-20K) was purchased from NANOCS (PG1-SVA-20K). Phosphate buffered saline (PBS) (28372) was purchased from ThermoFisher and prepared at 10× for further use. Gel loading dye blue (B7021S) was purchased from New England Biolabs and Sybr gold (S11494) was purchased from ThermoFisher. Centrifugal filters (UFC510096) for purifying seeds were purchased from MilliporeSigma. For cell culture, HeLa cell and HEK293 cell lines were both purchased form ATCC. DMEM medium (10-013-CV) was purchased from Corning Cellgro. FBS (26140079), 1% penicillin-streptomycin (15140122), 0.05% Trypsin-EDTA (25300054) and DPBS (14190144) were all purchased from ThermoFisher. For cell transfection, the Opti-MEM(1x) (31985062) was purchased from ThermoFisher and the X-tremeGENE (6365779001) form Sigma Aldrich. The azide-modified SpyTag peptide (Lot No. P3130-1) was synthesized by BioSynthesis. The size exclusion spin column Illustra Microspin G-25 was purchased from GE Healthcare. For AMDA, EGFR monoclonal antibody (H11) (MA513070), the Alexa fluor 647(A21236)-conjugated secondary antibody, the biotin-conjugated secondary antibody (31800), the streptavidin-Alexa Fluor 488 conjugate (S32354), neutravidin (31000) and DiD live cell labeling solution (V-22887) were all purchased from ThermoFisher. Integrin β1 Antibody (K-20) (sc-18887) (for integrin AMDA) was purchased from Santa Cruz Biotechnology. Bovine serum albumin (BSA) (A3858) and MgSO₄were purchased from Sigma-Aldrich. The Streptavidin Conjugation Kit (ab102921), used for conjugating streptavidin with Alexa 647 labeled secondary antibody, was purchased from Abcam. Borosilicate glass Lab-Tek 8-well chambers (155411PK) were purchased from ThermoFisher. Glass-bottom dishes (μ-Dish 35 mm, high Grid-50 glass bottom) (81148), μ-slide VI 0.4 (80606) and glass bottom μ-slide channel VI 0.5 (80607) were purchased from Ibidi. Biotin-PEG-silane (Biotin-PEG-SIL-3400-500 mg) was purchased from Layson Bio. Methylcellulose (HSC001) was purchased from R&D systems and Iscove's Modified Dulbecco's medium (IMDM) (12440053), which was used to dilute the methylcellulose, was purchased from ThermoFisher.

Synthesis of PEG-DNA strand conjugates. 8 mg NHS-PEG2OK was dissolved in 100 μL of a PBS buffer solution (pH 7.2) containing 50 μM amino-modified DNA strand. The mixture was agitated at room temperature (19-20° C.) overnight to allow the reaction to run to completion. Afterwards, the solution containing the PEG20K-DNA conjugates was loaded into a 7% PAGE gel. The running buffer was TAE-Mg²⁺ (40 mM Tris-acetate, 1 mM EDTA to which 12.5 mM magnesium acetate was added) and loading buffer 1× blue gel loading dye. The gel run at 150V for 1 h. The desired band containing the PEG-DNA conjugate was cut out and the conjugate was extracted from the gel by soaking the gel in water for 2-4 days to let the conjugate diffuse out from the gel. The concentration of the PEG20K-DNA conjugate was determined by quantifying the amount of Cy3-labeled PEG-DNA conjugate using fluorescence intensity and using a known concentration to quantitate the unlabeled conjugate via PAGE gel.

Assembly of PEG-coated DNA nanotube seeds. The structure and sequences of DNA nanotube seeds used herein are described in Example 4. To create PEG-coated DNA nanotube seeds, the staple sequences of a DNA origami seed structure were modified to each present a DNA sequence that served as an attachment site for a PEG-DNA conjugate (sequence AAGCGTAGTCGGATCTC (SEQ ID NO: 339)). The resulting seeds were assembled, purified and their concentrations measured using protocols adopted from Agrawal et al. (ACS Nano 11, 9770-9779, doi:10.1021/acsnano.7b02256 (2017), incorporated herein by reference). To coat the resulting nanotube seeds with PEG, 18 μL of a solution containing 10 μM PEG-DNA conjugate and 1.8 μL 10× TAE-Mg²⁺ buffer was added to 100 μL of a TAE-Mg²⁺ solution containing 0.8 nM seeds and incubated on the bench for 30 min.

Growing PEG-coated seeded nanotubes. The structures and sequences of DNA nanotube monomers with both 6- and 4-base sticky end binding sites are given in Example 4. To grow PEG-coated seeded nanotubes with 6 base sticky ends, the central SEs3 strand of the tile was conjugated with PEG as described in Example 4. 19.7 μL of a TAE-Mg²⁺ solution containing 450 nM of each of the strands for the monomers were annealed from 90 to 37° C. 2 μL of a solution containing 0.4 nM PEG-coated seeds was added after the monomer solution reached 37° C. The mixture was kept at 37° C. for 3 days. To grow 4PEG nanotubes, the central REd3 and SEd3 strands of the two monomer types were each conjugated with PEG. 19.7 μL of a TAE-Mg²⁺ solution containing 180 nM of each the strands of the two 4PEG nanotube monomers was annealed from 90 to 20° C. as described in Agrawal et al. (ACS Nano 11, 9770-9779, (2017), incorporated herein by reference). 2 μL of a TAE-Mg²⁺ solution containing either 0.4 nM PEG-coated anchored nanotube seeds or 0.4 nM PEG-coated capped nanotube seeds as appropriate for experiments on nanotube joining were added after the solution reached 20° C. The solution was then incubated at 20° C. for 3 days.

Cell culture. HeLa cells and HEK 293 cells were grown in DMEM medium containing 10% FBS and 1% penicillin-streptomycin. 5 mL of culture was grown in 25 cm² culture flasks at 37° C. in 5% CO₂ and constant humidity. Cells were released from the flask surface using 0.05% Trypsin-EDTA and split every two days. The HeLa cells were cultured in media with: 3, 6, 9, and 12 mM MgSO₄ overnight, after which time cell viability was confirmed by shape under a bright-field microscope.

Characterizing the extent of nonspecific interactions between DNA nanotube seeds or seeded nanotubes and HeLa cells. HeLa cells were seeded in borosilicate glass Lab-Tek 8-well chambers at a density of 40000 cells per well in 250 μL medium. DNA nanotube seeds with and without PEG coating were diluted to make 8, 16, 32 and 64 pM solutions in a cold DMEM solution containing 1% BSA (w/v) and 12 mM MgSO₄. The medium in each well chamber containing HeLa cell was exchanged for 250 μL of diluted seeds solution. The cells were then incubated in a 4° C. refrigerator for 30 minutes and subsequently washed with DMEM-12mM MgSO₄ buffer 3 times. Seeded DNA nanotubes grown from 37 pM seeds with PEG or 28 pM seeds without PEG in TAE-Mg²⁺ were diluted one-fold with cold 1% BSA(DMEM)-12mM MgSO₄. The medium in the wells containing HeLa cells was exchanged for diluted nanotube solution. The cells were then incubated in a 4° C. refrigerator for 2 hours and subsequently washed with DMEM-12mM MgSO₄ 3 times. After both treatments, the cells were fixed using 4% paraformaldehyde before imaging.

Transfection of HeLa cells with SpyCatcher-fusion transgenes. The GFP-integrin-SpyCatcher plasmids were constructed by inserting the SpyCatcher DNA sequence into the GFP-integrin construct at the NotI restriction site. The backbone of the plasmid is a Clontech vector with kanamycin resistance. The plasmid was transformed and amplified in DH5alpha bacteria, and purified using a Qiagen miniprep kit. HeLa cells for transfection were cultured and passaged as described above. HeLa cells were counted using a hemacytometer and diluted to 2.4×10⁵ cell per mL in DMEM. For each transfection process, 38 μL of Opti-MEM (1×) and 2 μL of a solution containing 1 mg/mL plasmid DNA were mixed well in a 1.5 mL Eppendorf tube via pipetting. 2 μL X-tremeGENE 9 solution was then added and the solution again mixed well via pipette, after which the mixture was incubated at room temperature for 30 minutes. 42 μL of the plasmid solution was then added to 500 μL of diluted cells in a 1.5 mL Eppendorf tube and mixed well by gently inverting the tube around 20 times. 250 μL of cell solution was then pipetted into a well of borosilicate glass Lab-Tek 8-well chamber. The cells were then incubated for 2 days before use.

Attachment of seeded nanotubes to cells using SpyCatcher-SpyTag attachment. SpyTag-modified seeded DNA nanotubes were prepared by attaching an azide-modified SpyTag peptide to an amino-modified DNA oligonucleotide via a click reaction and hybridizing this strand to the complementary sequence presented at the ends of nanotube seeds which were then spin-filtered to remove extra SpyTag. Nanotubes were then grown from seeds with and without the SpyTag modification and diluted 1.5-fold with DPBS-12 mM MgSO₄ buffer. The medium in each well chamber containing HeLa cells expressed with GFP-integrin-SpyCatcher fusion protein was exchanged for 250 μL of diluted nanotube solution prepared as described for characterization of nonspecific nanotube-cell interactions but diluted by DPBS with 12 mM MgSO₄ buffer and incubated at 37° C./5% CO₂ for 30 minutes.

Attachment of nanotube seeds or seeded nanotubes to EGFR receptors on HeLa cells using AMDA. PEG-coated nanotube seeds or seeded nanotubes were prepared as described as above and HeLa cell were seeded overnight in borosilicate glass Lab-Tek 8-well chambers at a density of 40000 cells per well with 250 μL medium. The next morning, the cells were first incubated in a 4° C. refrigerator for 10 minutes. DMEM buffer containing 1% BSA (w/v) was then added to the cells, which were then incubated for 5 minutes. To attach nanotube seeds, 250 μL of solution containing 1) 2μg/mL EGFR primary antibody, 2) 10 μg/mL Alexa 647-labeled secondary antibody-streptavidin conjugate (Alexa 647 2AB-STA), 3) 1 μM BDC tag and 4) BDC′ tag-labeled nanotube seeds at concentration to achieve the stated concentration in cell solution were each added to the cells in the order listed. After each addition the cells were incubated in the refrigerator for 30 minutes then washed 3 times with cold DMEM (DMEM-12mM MgSO₄ after seeds or nanotubes were added) to remove reagent not attached to the cells. To attach seeded nanotubes, the Alexa 647-labeled secondary antibody solution was replaced by 250 μL solutions containing 2a) 500-fold diluted biotinylated secondary antibody followed by 2b) 3 μg/mL Alexa488-labeled streptavidin. After each addition cells were washed 3 times with cold DMEM buffer. To attach nanotubes to cells, the seed solution was replaced by a one-fold diluted solution of seeded nanotubes. This solution was incubated with cells for 2 hours, pipetting gently every 30 minutes. After incubation cells were washed with DMEM-12 mM MgSO₄ buffer 3 times.

Attachment of nanotube seeds or nanotubes to EGFR receptors on HEK293 cells using AMDA. PEG-coated nanotube seeds and seeded nanotubes were prepared as described as above and HEK293 cells were trypsinized and suspended to a concentration of 10⁶-10⁸ cells per mL. The cells were centrifuged at 300RCF for 5 minutes, the supernatant was removed and the cells were resuspended in cold 1% BSA in DMEM. To attach nanotube seeds to suspended HEK293 cells, the cells were centrifuged and resuspended in a solution containing 1) 2 μg/mL EGFR primary antibody, 2) 10 μg/mL Alexa 647 2AB-STA 3) 1 μM BDC tag and 4) PEG-coated nanotube seeds in sequence. After each addition, the cells were incubated in a 4° C. refrigerator for 30 minutes and pipetted-mixed every 15 minutes, then washed by centrifuging and resuspending in 1 mL cold DMEM buffer to remove unattached reagent. After this sequence, the cells were resuspended in DMEM-12 mM MgSO₄ buffer. To attach seeded nanotubes, resuspension in Alexa 647-labeled secondary antibody solution was replaced by resuspension in 2a) 500-fold diluted biotinylated secondary antibody then 2b) 3 μg/mL Alexa488-labeled streptavidin. After the addition of the BDC tag and resuspension, the cells were resuspended in 1 mL cold DMEM buffer then a solution of seeded nanotube solution (1-fold diluted after preparation). The cells were incubated in a 4° C. refrigerator for 2 hours during which time they were pipetted gently every 30 minutes. The cells were centrifuged a last time, then resuspended in DMEM-12 mM MgSO₄ buffer.

Attachment of nanotubes to integrin receptors on HeLa cells using AMDA. The steps for AMDA were followed above except that the EGFR primary antibody solution was replaced with a solution containing 4 μg/mL Integrin β1 antibody. After addition, cells were incubated at 4° C. for 1 hour.

Spinning disk confocal microscopy. Cells with attached DNA nanotube seeds or seeded nanotubes were imaged using a Zeiss AxioObserver Yokogawa CSU-X1 spinning disk confocal microscope with a 60× oil objective. Stack images were taken from the bottom of the cell to the top of the cell with a stack depth of 0.27 μm (for HeLa cells) or 0.5 μm (for HEK293 cells) at 10-15 random locations.

Quantification of the number of nanotube seeds and seeded nanotubes attached to each cell. The average fluorescence intensity per cell was used to quantify the average number of seeds/seeded nanotube attached to a HeLa cell. A z-stack of images was collected at each imaging position, and the stack image from the height closest to the center of an average-sized cell was selected for analysis. This choice was made because this image largely excluded the structures attached to the glass rather than the cell while maintaining a sufficiently large cross-sectional area for each cell for analysis. The number/total length of nanotube seeds and seeded nanotubes present was measured by characterizing the total fluorescence intensity. The seeds' intensity per cell and nanotube intensity per cell were then calculated by dividing these respective quantities by the number of cells in an image, which was also counted manually. Flow cytometry (BD FACSCanto) was used to characterize the number of nanotube seeds on the HEK293 cells. The number of seeded nanotubes on HEK293 cells was determined by generating a 3-dimensional projection image from each of a z-stack of images collected at multiple random locations and manually counting the number of seeded nanotubes visible on each cell. The amount reported is the average of these counts.

Quantification of the dwell time of seeds and seeded nanotubes on the cell membrane after AMDA. PEG-coated DNA nanotube seeds or seeded nanotubes were anchored on HeLa-GFP cells using EGFR AMDA as described above. The cells were washed with cold (4° C.) fresh DMEM medium containing 10% FBS, 1% penicillin-streptomycin and 12 mM MgSO₄ then placed into an incubator (37° C., 5% CO₂ and constant humidity) on a Nikon Al confocal microscope (Nikon, Tokyo, Japan) with a 63× oil objective. Stacks of images at heights spanning the bottoms and tops of the cell in the field of view. Image stacks of cells with attached nanotube seeds were collected every 10 minutes over 70 minutes with a stack height of 0.27 Image stacks of cells with attached nanotubes were collected every 15 minutes over 20 hours with a stack height of 1 μm. The number of nanotube seeds and seeded nanotubes on each tracked cell's membrane were counted manually. These numbers were normalized by the number of seeds or nanotubes on that cell at t=0. Seeds were counted if they were visible at a cell's edge. The top and bottom images of a stack were omitted from quantification because it was not possible to determine whether seeds were beneath or above the cell rather than at the cell surface. The total number of seeded nanotubes on a cell's surface at each time point was manually counted using maximum projection images.

Use of fluid flow to apply shear stress at a glass surface or HeLa cell membrane. Shear stress was applied within in flow cells (Ibidi, μ-slide VI 0.5 for glass-anchored nanotubes and μ-slide VI 0.4 for cell-anchored nanotubes) and the shear stress corresponding to a given flow rate was calculated according to the methods provided by Ibidi. The flow rates applied to the seeded nanotubes anchored to glass surfaces and to cells and their respective expected shear stresses are shown in Table 1.

SUPPLEMENTARY TABLE 1
Fluid flow shear stress and flow rate for nanotubes
on cell membranes and glass surfaces

| | Q in μ-slide VI 0.4 | Q in μ-slide VI 0.5 |
|---|---|---|
| τ | (nanotubes on cells) | (nanotubes on glass) |
| (dyn/cm²) | (mL/min) | (mL/min) |
| 0.05 | 0.03 | 0.05 |
| 0.11 | 0.06 | 0.10 |
| 0.21 | 0.12 | 0.20 |
| 0.42 | 0.24 | 0.40 |
| 0.63 | 0.36 | 0.61 |
| 0.85 | 0.48 | 0.81 |
| 1.1 | 0.6 | 1.0 |
| 1.3 | 0.72 | 1.2 |
| 1.7 | 0.96 | 1.6 |
| 2.1 | 1.2 | 2.0 |

Seeded nanotubes were anchored to the glass bottoms of flow cells using a method developed previously (Mohammed, A. M., et al., Nature Nanotechnology 12, 312, 2016, incorporated herein by reference). A syringe pump (New Era, NE-1000) was used to induce controlled, unidirectional laminar flow. TAE-Mg²⁺ buffer was used as flow perfusate. A series of flow rates in order from lowest to highest were applied to the nanotubes attached to the glass as follows: 0, 0.05, 0.10, 0.20, 0.40, 0.61, 0.81, 1.01, 1.21, 1.62 and 2.02 mL per minute. Seeded nanotubes were anchored to the HeLa cell membrane through EGFR AMDA performed in a flow cell. DMEM-12mM MgSO₄ buffer was used as flow perfusate. A series of flow rates in order from lowest to highest were applied to the cells as follows: 0, 0.03, 0.06, 0.12, 0.24, 0.36, 0.48, 0.6, and 0.72 mL per min. Seeded nanotubes under fluid flow were imaged using a spinning disk confocal microscope with 5 seconds intervals for 30 cycles. The nanotubes were imaged in the xy-plane in which the largest number of seeds and the largest fraction of the nanotubes were in focus. The total angle of the nanotube rotation under fluid flow was measured by first cropping the area spanned by a single nanotube from each larger image. A Gaussian blur filter (radius:1.00) was applied in ImageJ for all the 30 cropped images to reduce the image background. A maximum time projection image was then generated from this cropped time-lapse movie and the total angle of the nanotube rotation under fluid flow was measured manually. The total angles of 15 nanotubes on the glass surface and >15 nanotubes on cell membrane were measured for each shear stress.

Growth of seeded nanotubes anchored to the cell membrane. 4PEG nanotubes and 4PEG capped and seeded nanotubes were prepared as described elsewhere herein. Here both the capped and anchored nanotube seeds were unlabeled. Anchored seeded nanotubes were attached to the HeLa cell membrane through EGFR AMDA within μ-slide channel. 50 μL of a solution containing 900 nM inactive nanotube monomers (FIG. 11B) labeled with atto647 was annealed in TAE-Mg²⁺ buffer from 90 to 20° C. 0.27 μL of solution containing 100 μM of a strand to activate the inactive monomers was added to this solution after which 25 μL of it was immediately mixed with 60 μL of a solution containing 37 pM of capped nanotubes and 65 μL of DMEM-12.5 mM MgSO₄ buffer containing 1% BSA. This mixture was immediately added to the HeLa cells to which seeded nanotubes had been attached. The sample was covered with foil and incubated on the lab bench (at about 19˜21° C.) for 4 h. It was then washed with DMEM-12mM MgSO₄ three times before imaging. A gentle fluid flow (0.18 mL/min) inducing a shear stress of 0.32 dyn/cm² was applied to stretch the nanotubes and allow visualization of their contours. An epi-fluorescence microscope with a 60× oil objective was used to capture continuous 20 images of each location. The yield of nanotube joining on cell membrane was calculated by counting the total number of seeded nanotubes on the cell and the number of nanotubes on the cell that had visually joining. Cells in six images were quantified. 0.6% methylcellulose media (IMDM) with 12 mM MgSO₄ was added to HeLa GFP cells after the joining protocol (but not flow or imaging) was completed. Stacks of images at random locations were taken from the bottoms to tops of cells with a stack height of 0.27 μm using a spinning disk confocal microscope.

### Example 1

### Anchoring Nanotube Seeds to Cell Receptors

The design for anchoring DNA nanotubes at their ends to specific receptors on living cells tailored to target many different receptor types presents key challenges. First, a nanometer-scale anchor point on a filament's end must direct binding specifically to chosen receptor, but the filament's much larger remaining surface must not interact strongly with the cell. The anchoring rate must also be higher than the rates of nanotube detachment or cell import. Microparticles can be anchored to cells because their large surface areas means high net attachment rates; molecules or complexes can be reliably anchored when they are supplied at high concentrations (>>10 nM)³⁴. But because of their large size (˜50 megadaltons), it is only practical to present DNA nanotubes at concentrations <100-200 pM. To overcome these challenges, a method in which a DNA nanotube seed serves as an anchor and presents numerous binding sites that attach quickly and effectively irreversibly at the desired receptor was developed. This approach yielded efficient attachment with little background and works for multiple receptor and cell types.

Nonspecific interactions between DNA nanotube seeds and nanotubes and cells, which have been observed previously and may be the rest of electrostatic interactions between negatively charged DNA and positively charged groups on cells were characterized and eliminated. To measure the rate of DNA nanotube seed/cell interaction Atto488-labeled DNA nanotube seeds (final concentrations 8-64 pM) were added to HeLa cells in culture. Confocal micrograph z-stacks at fifteen random locations showed the average fluorescence intensity of seeds at the cells' midline increased linearly with seed concentration, with 107±17 attached seeds per cell for 64 pM seeds.

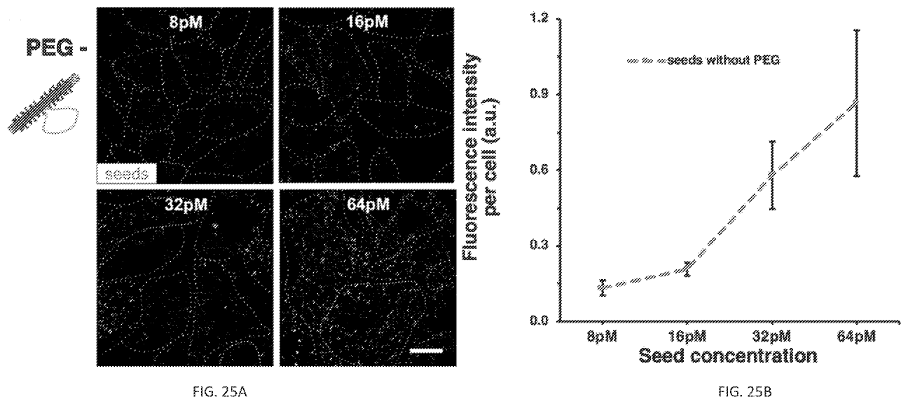

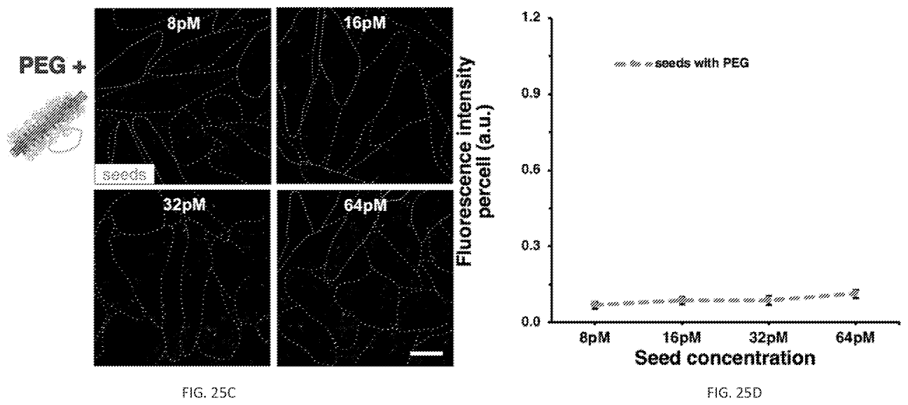

Poly(ethylene) glycol (PEG) coating can reduce nonspecific interactions between nanoparticles and cell membranes. To test whether PEG coating might reduce nonspecific interaction between DNA nanostructures and cells, 20 kD PEG-15 nt DNA strand conjugates were hybridized to seeds (FIGS. 1D, 14 and 15). Almost no PEG-coated seeds were visible on cells after 8-64 pM PEG-coated seeds were incubated with HeLa cells (FIG. 25).

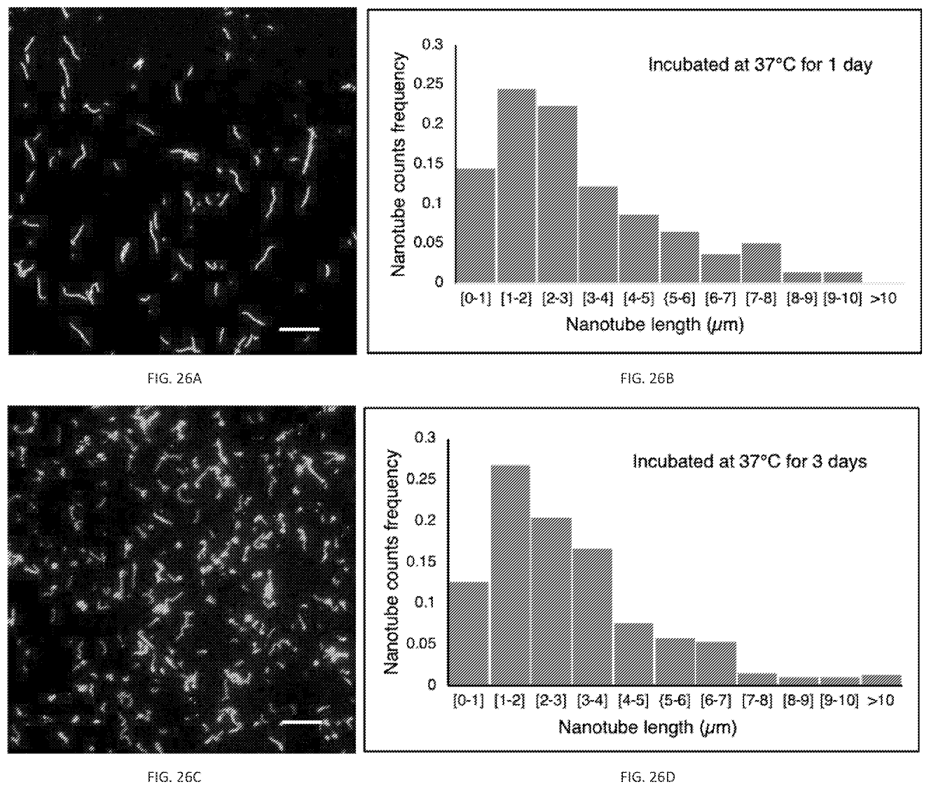

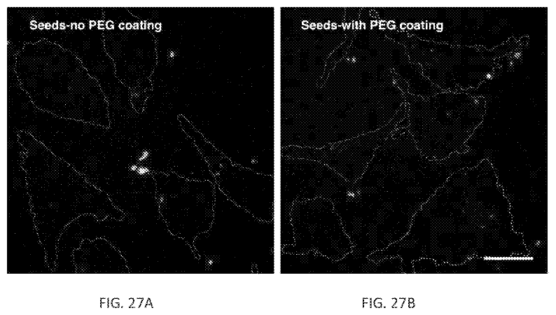

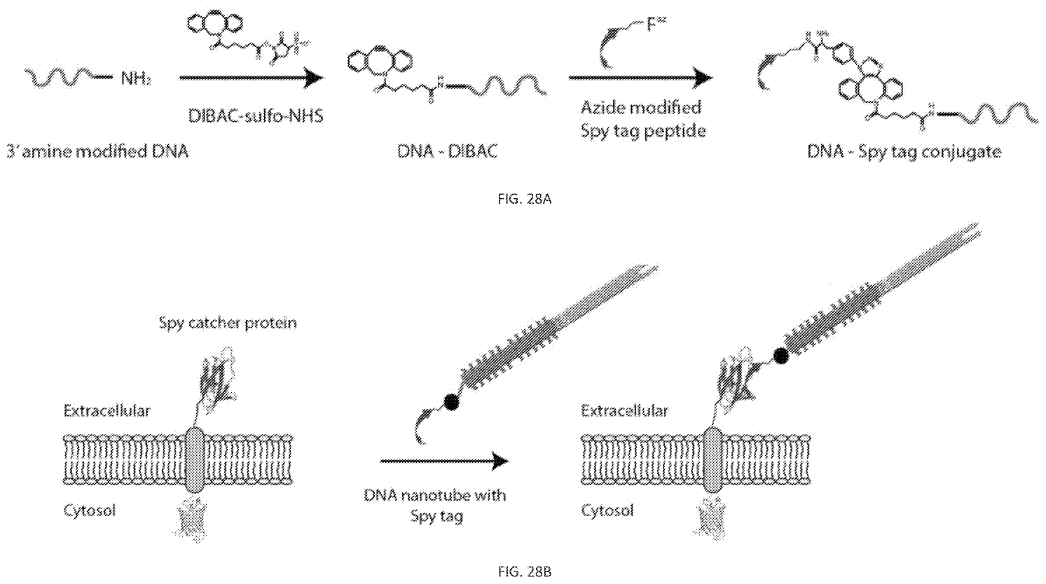

To prevent nonspecific interactions between DNA nanotubes and cells, 20 kD PEG was conjugated to nanotube monomers (FIG. 1C), then seeded nanotubes were prepared by combining 415 nM PEG-conjugated monomers with 37 pM seeds and incubating them at 37° C. in TAE-Mg²⁺ buffer for 3 days. Greater than 40±4.8% of the resulting filaments were >3 μm long (FIG. 26). Neither PEG-coated nanotubes grown from PEG-coated or unmodified nanotube seeds attach to cells (FIG. 27).

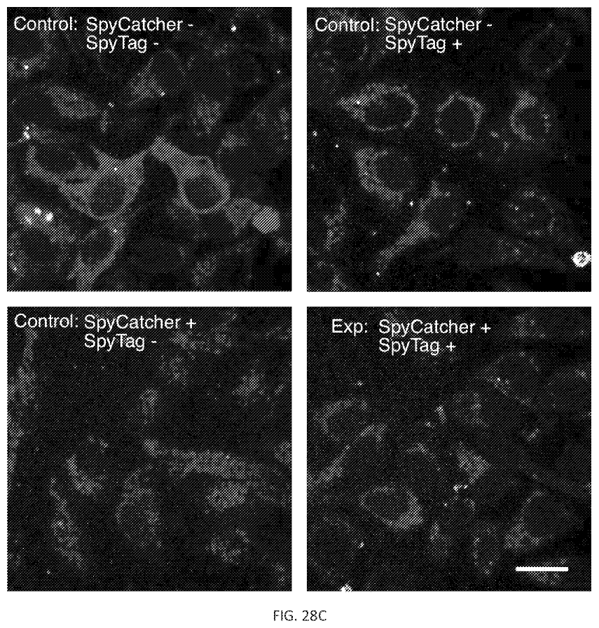

Anchoring DNA nanotube seeds to cells was first attempted using the SpyTag peptide and SpyCatcher protein, which form a covalent bond. Six SpyTag peptide-DNA conjugates were hybridized to each seed's barrel. A GFP-integrin-SpyCatcher fusion protein was expressed in HeLa cells via transfection. However, almost no nanotubes grown from SpyTag-modified seeds attached to cells (FIG. 28), perhaps due to the low SpyTag-SpyCatcher reaction rate constant: 1400±40 M⁻¹s⁻¹.

Even assuming fusion receptor overexpression (10⁴ per cell), on average just one nanotube would anchor to each cell if 64 pM nanotubes were added. Realizing that anchoring nanotubes required a much faster binding reaction, antibody-receptor interactions were considered, as most protein interactions have forward rate constants of 10⁵-10⁶ M⁻¹s⁻¹.

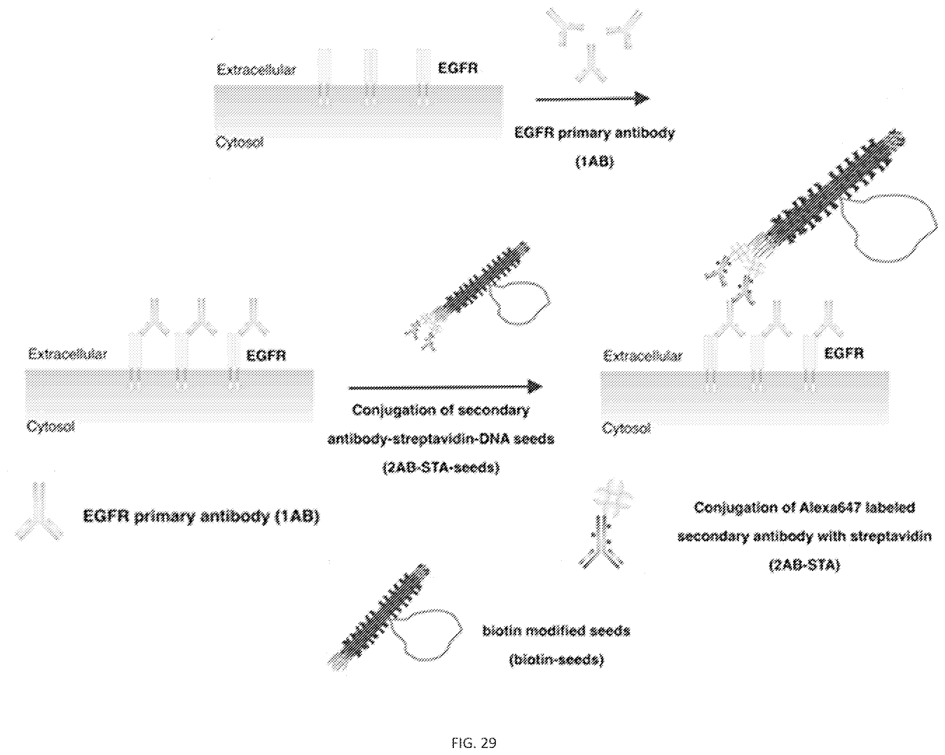

Anchoring DNA nanotubes to epidermal growth factor receptors (EGFR) was attempted using EGFR-EGFR antibody binding. EGFR is a transmembrane receptor tyrosine expressed at up to 10⁶ copies per HeLa cell. Fluorescently labeled secondary antibodies attached to fixed and live HeLa cells only after EGFR primary antibodies were added. However, only a few DNA origami seeds with six secondary antibodies on their barrels attached to an average cell to which primary EGFR antibodies had been added (FIG. 29). While receptor-antibody interactions were likely fast enough for hundreds of seeds to attach, most antibodies (including the EGFR antibody⁴³) have >nM affinities. DNA seeds, present at only picomolar concentrations, did not remain attached on average, indicating stronger binding was needed.

Forward rate constants of DNA hybridization are 10⁵-10⁶ M⁻¹s⁻¹, and a 15-nt DNA strand binds to its complement with sub-picomolar affinity under physiological conditions. A process in which a DNA sequence, termed the biotin-DNA connection (BDC) tag was designed. In this process the BDC tag was first attached to a receptor using antibodies, biotin and neutravidin. Nanotube seeds present a BDC tag complement (BDC′), which hybridized to the tethered BDC (FIG. 1E). This approach may be generalized to attach different structures to different cell receptors: different antibodies could present different BDC sequences and different nanostructures their respective complements. This process is referred to herein as “antibody-mediated, DNA-directed attachment” (AMDA).

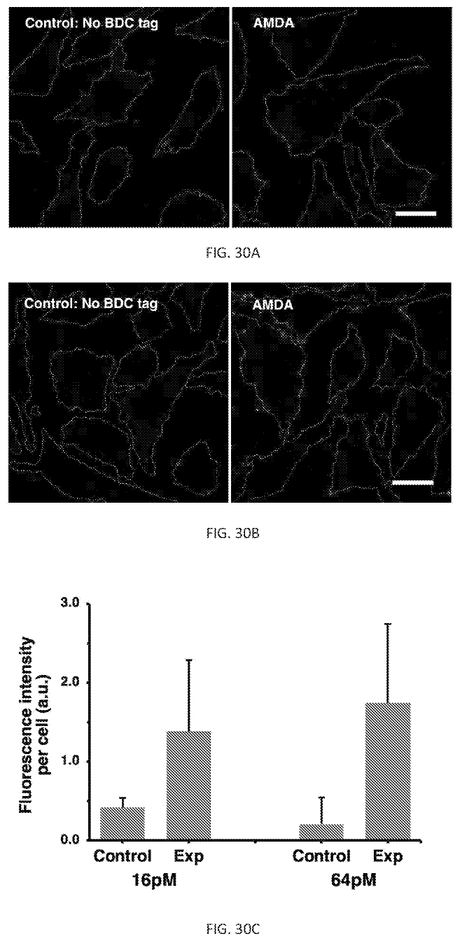

To test AMDA, greater than 10 nM of each of primary EGFR antibodies, biotinylated secondary antibodies, neutravidin and BDC tag were added stepwise to live HeLa cells. 16 or 64 pM nanotube seeds were then added. About 2-fold more seeds attached to cells after either 16 pM or 64 pM seeds presenting 6 BDC′ sequences at their barrels' ends were added than after a control AMDA process where the BDC strand was not added (FIG. 30) fewer than 10 seeds were attached per cell.

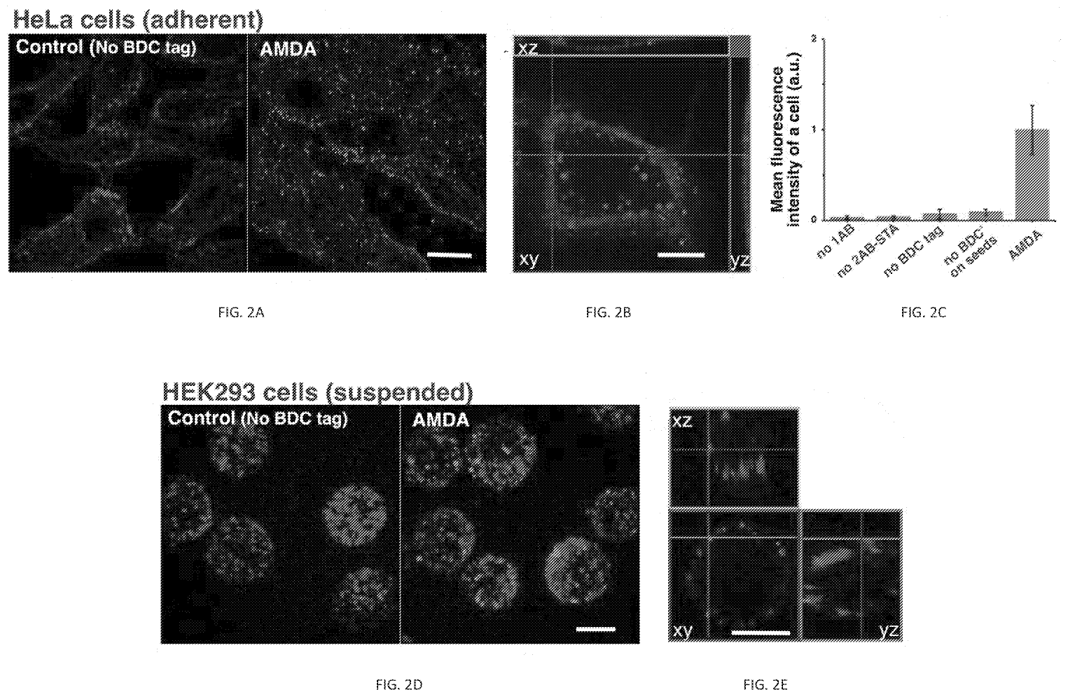

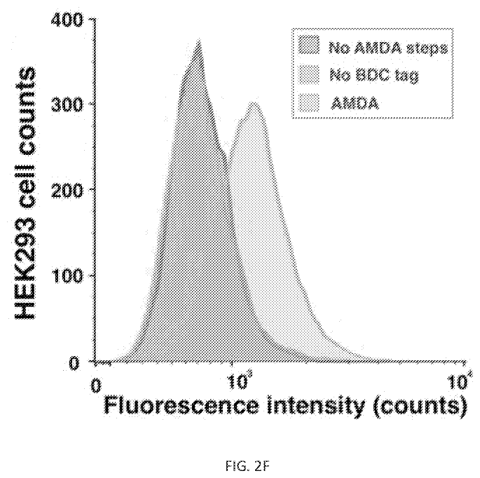

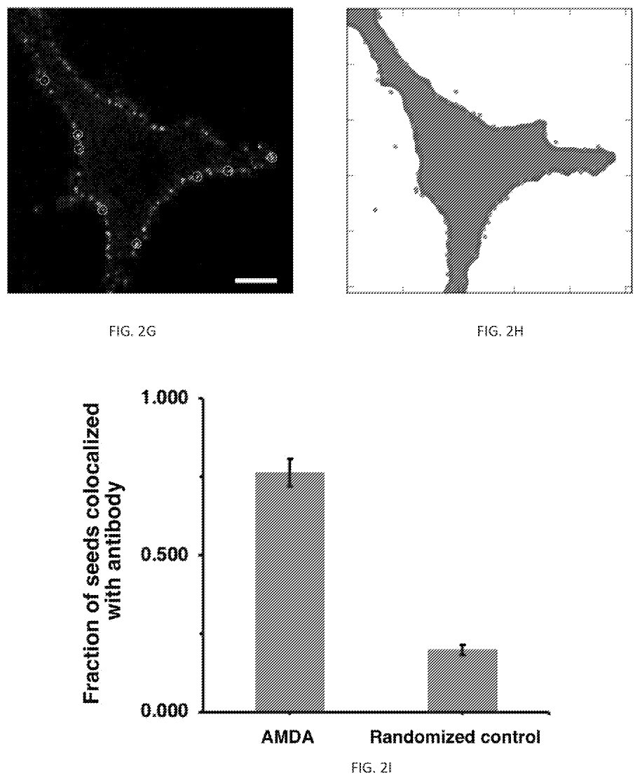

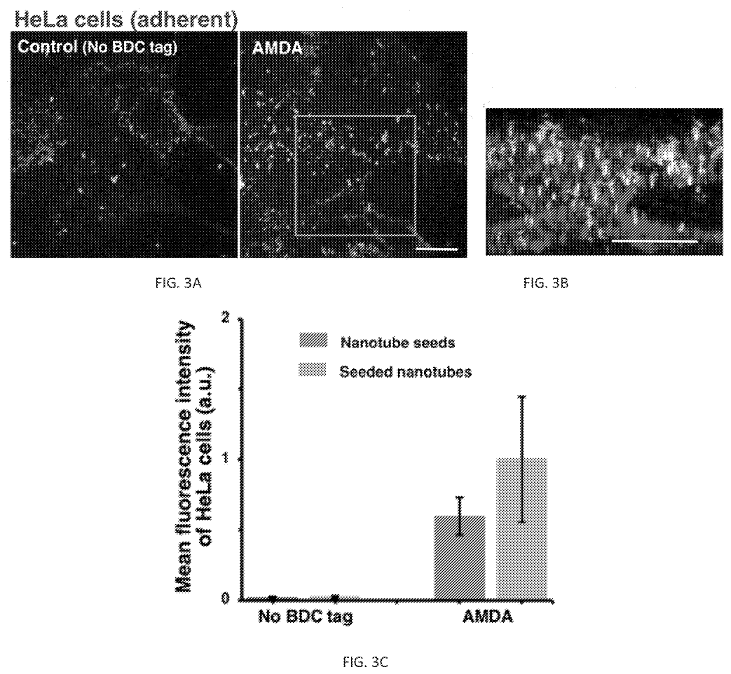

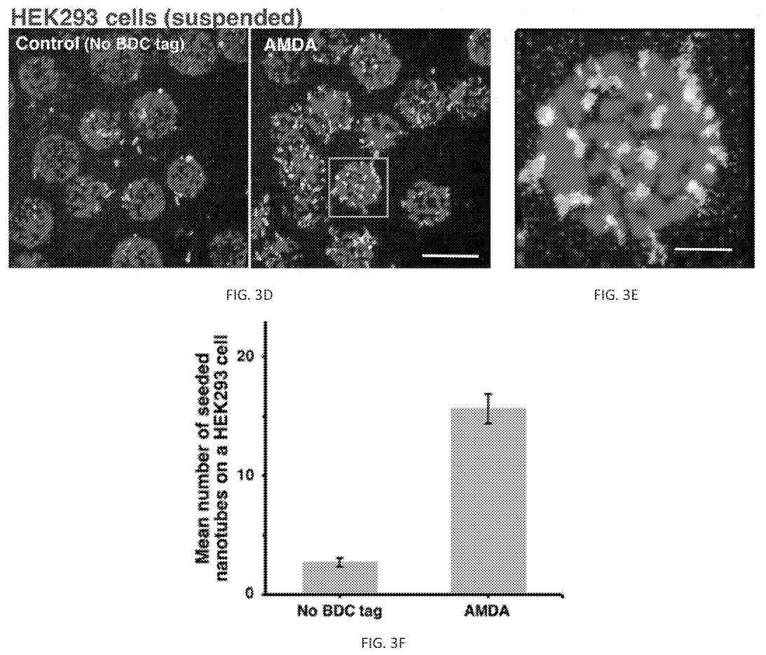

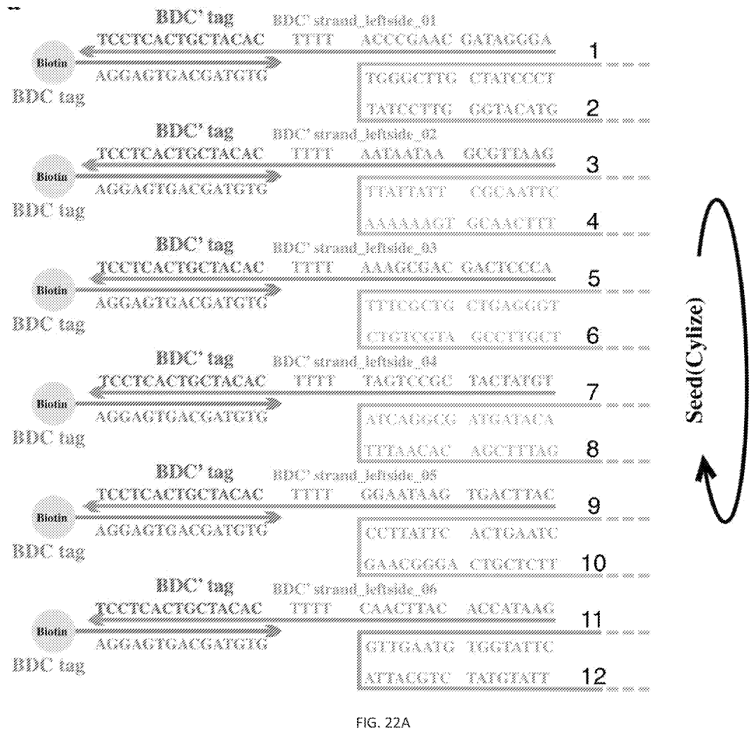

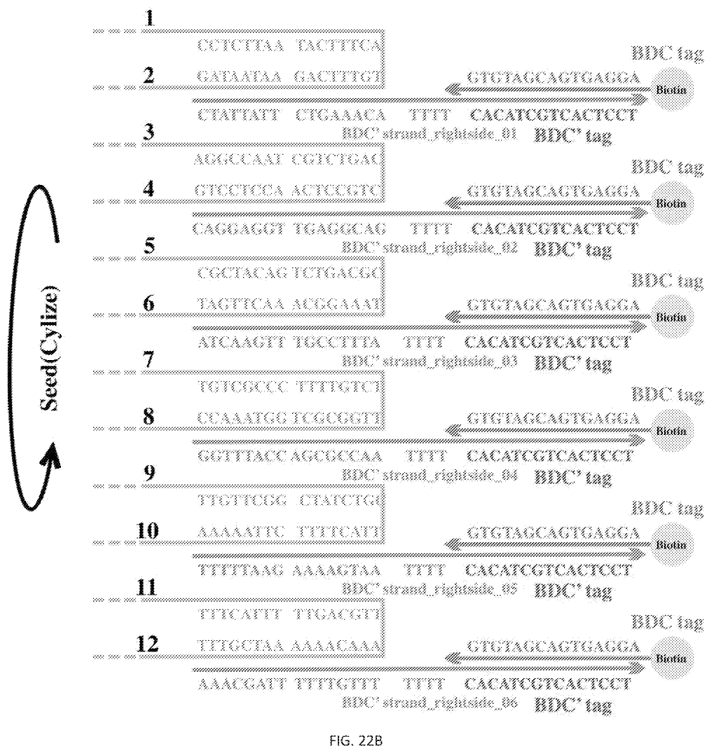

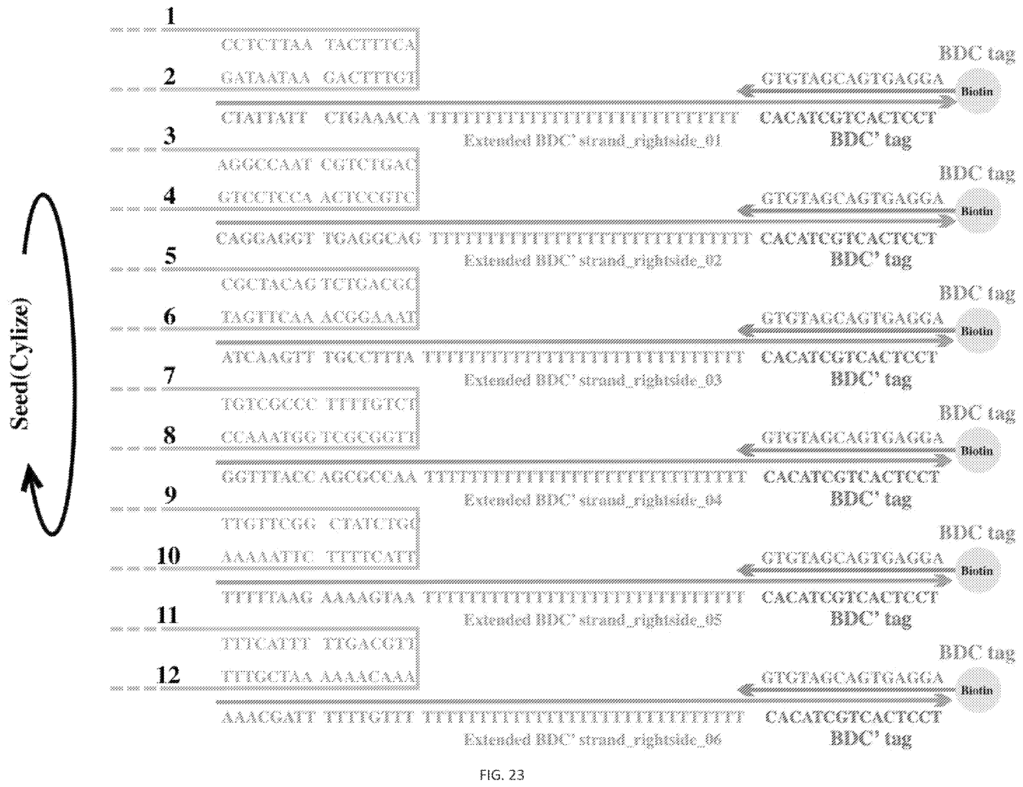

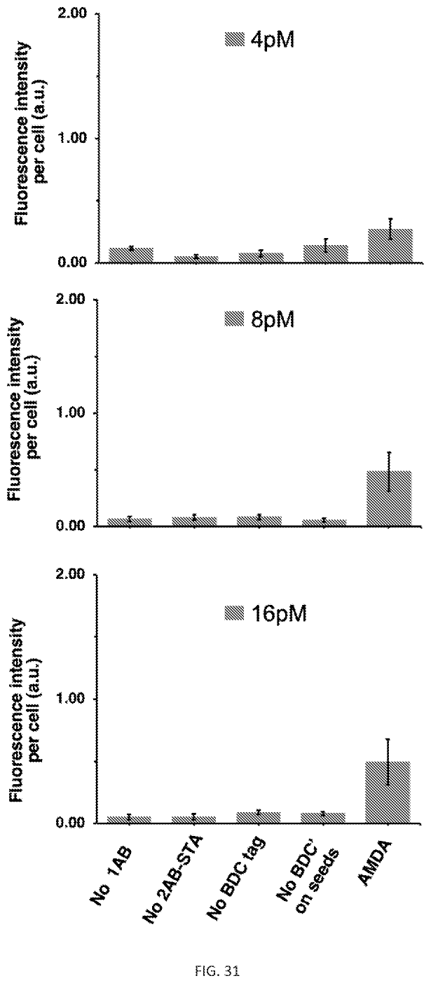

The PEG coating on the seeds may cover the BDC′ sequences. Twenty-four thymines were added to the BDC′ presenting strands to increase the distance between the BDC′ sequence and the PEG. Thirty fluorescently labeled DNA strands attached to a loop of DNA were replaced on the seed (FIG. 1D and 21) with strands presenting the BDC′ sequence. These changes dramatically increased the number of seeds attached to HeLa cells after AMDA without increasing nonspecific attachment (FIG. 2A). Cross-sectional images showed seeds on the cell membrane, consistent with receptor attachment (FIG. 2B). Elimination of any AMDA step resulted in almost complete elimination of seed attachment (FIG. 2C and 31).

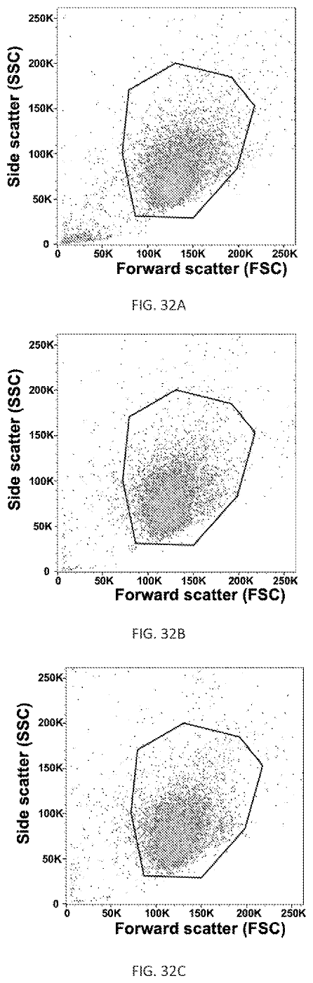

AMDA was used to attach nanotube seeds to EGFR on suspended HEK293 cells. Nanotube seeds were present all over cells, and little attachment was observed in controls (FIG. 2D). The fluorescence intensity over background of HEK293 cells as determined by flow cytometry (674±75) was >5-fold higher after seeds were attached by AMDA than after a control process (125±55) (FIG. 2F and 32).

To verify that DNA seeds attached proximal to EGFR, the colocalization of nanotube seeds with fluorescently labeled EGFR antibodies was measured. 76±4% (N=12 cells) of seeds were colocalized with EGFR antibodies after AMDA, whereas random chance would result in only 20±2% (N=12 cells) colocalization (FIG. 2G-I).

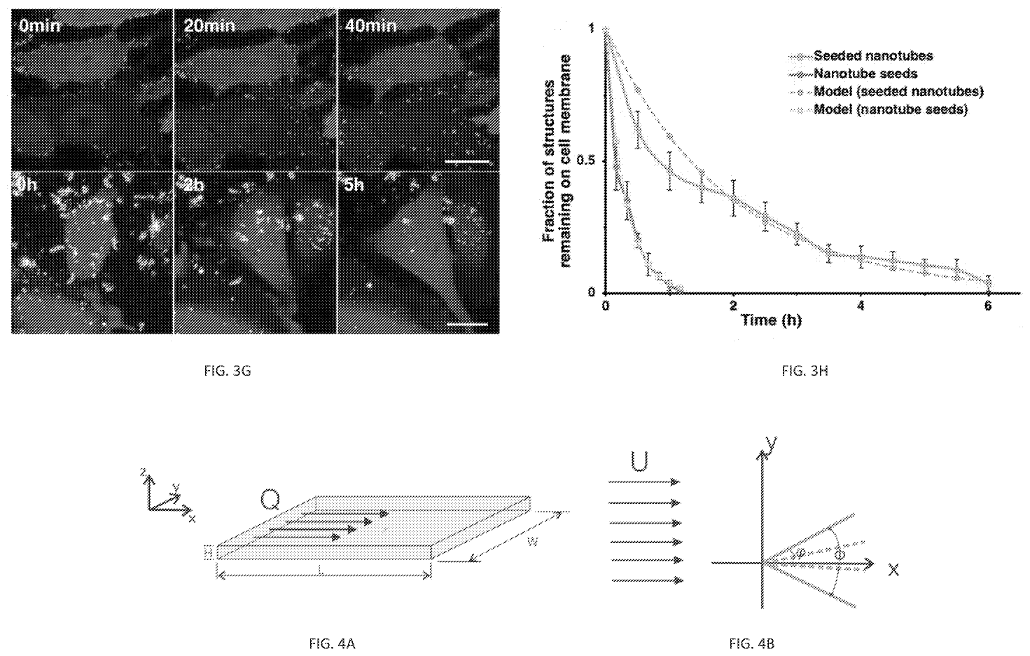

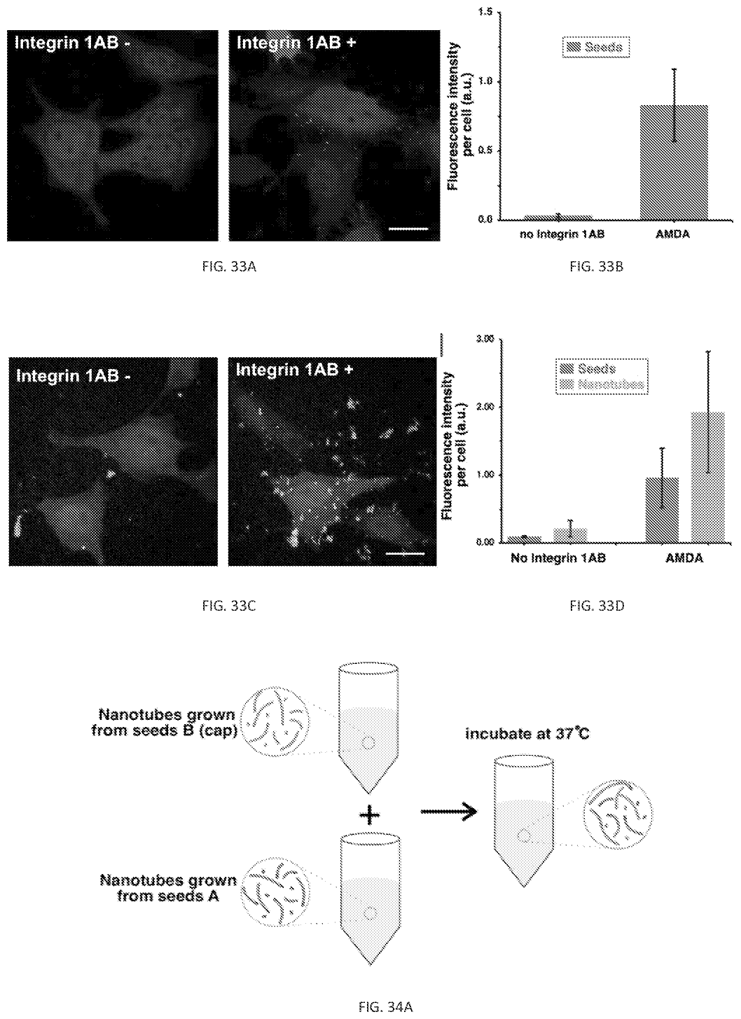

Seeded DNA nanotubes attached reliably to HeLa cell membranes via AMDA but not in controls (FIGS. 3A-B). Cells were 35-fold more fluorescent in the nanotube seed channel (Atto488) and 45-fold more fluorescent in the nanotube (Cy3) channel over background after AMDA vs. after a control. Seeds did not move in time-lapse movies, but attached nanotubes moved freely, indicating that nanotubes were anchored to cells by seeds. Nanotubes could also be anchored to EGFR on HEK293 cells (FIGS. 3D-F). Nanotubes could be anchored to integrin receptors on HeLa cells via AMDA, demonstrating AMDA's generality (FIG. 33).

As receptor turnover or seed endocytosis could lead to detachment or import, how long nanotubes or nanotube seeds could persist on a cell's surface at 37° C. was investigated. DNA origami seeds and seeded nanotubes were first attached to HeLa-GFP cells at 4° C., where detachment rates were low. The cells were then returned to 37° C. where decreases in the number of seeds or nanotubes on the cell surface were measured using time-lapse confocal microscopy. The fractions of nanotubes and nanotube seeds on the surface both decreased exponentially with time (FIGS. 3G-H), seeds 6 times faster than seeded nanotubes (FIG. 3H). These measurements did not distinguish whether structures detached from or were imported into the cell. EGFR-mediated endocytosis is a receptor-mediated clathrin-dependent pathway in which a membrane invagination is pinched off by the motor protein dynein. Nanotube seeds (FIG. 1D, length: 65nm) are small enough to conceivably be endocytosed with EGFR. EGFR-mediated endocytosis takes on order 30 minutes, consistent with the rate of seeds leaving the cell surface. Nanotubes are too large to be endocytosed, but dynein-controlled membrane closure could sever them. EGFR is a fast-turnover receptor and HeLa cells are fast-growing cells so these persistence times are likely at the lower range across different receptors and cell lines.

### Example 2

### Nanotube Shear Stress Sensors

Shear can result from flow and is a key environmental signal in vivo. For example, the primary cilium is involved in sensing flow in the kidney, and bends in response to flows, inducing signaling. Nanotubes might bend in response to shear stress and the extent of this bending might indicate the magnitude of shear stress.

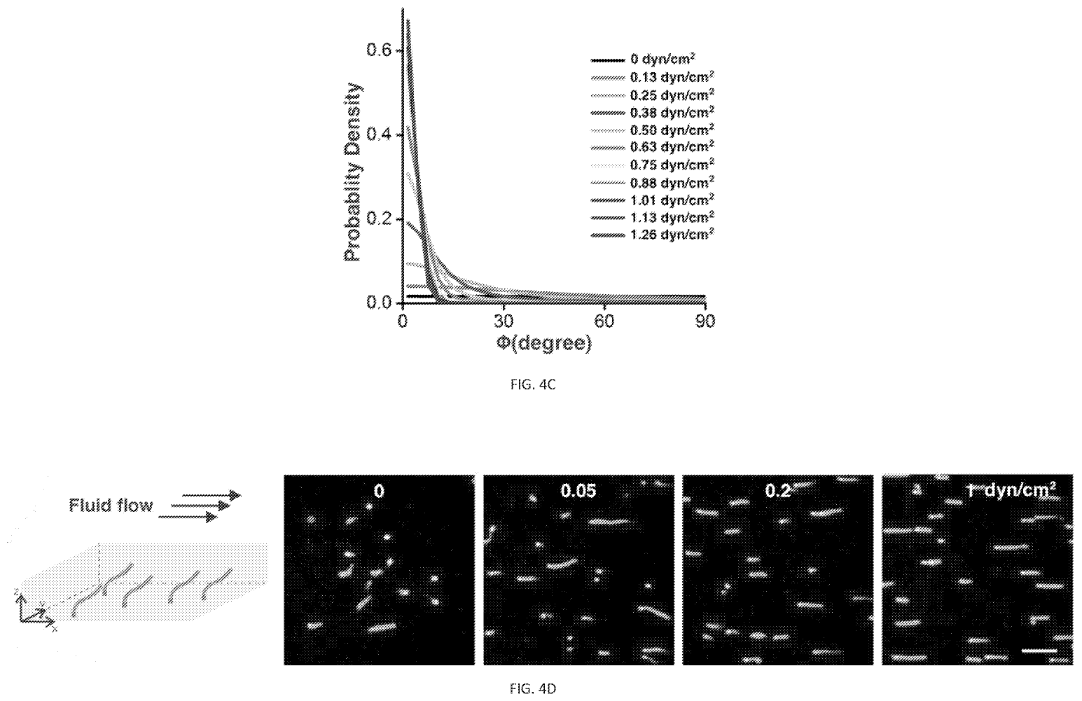

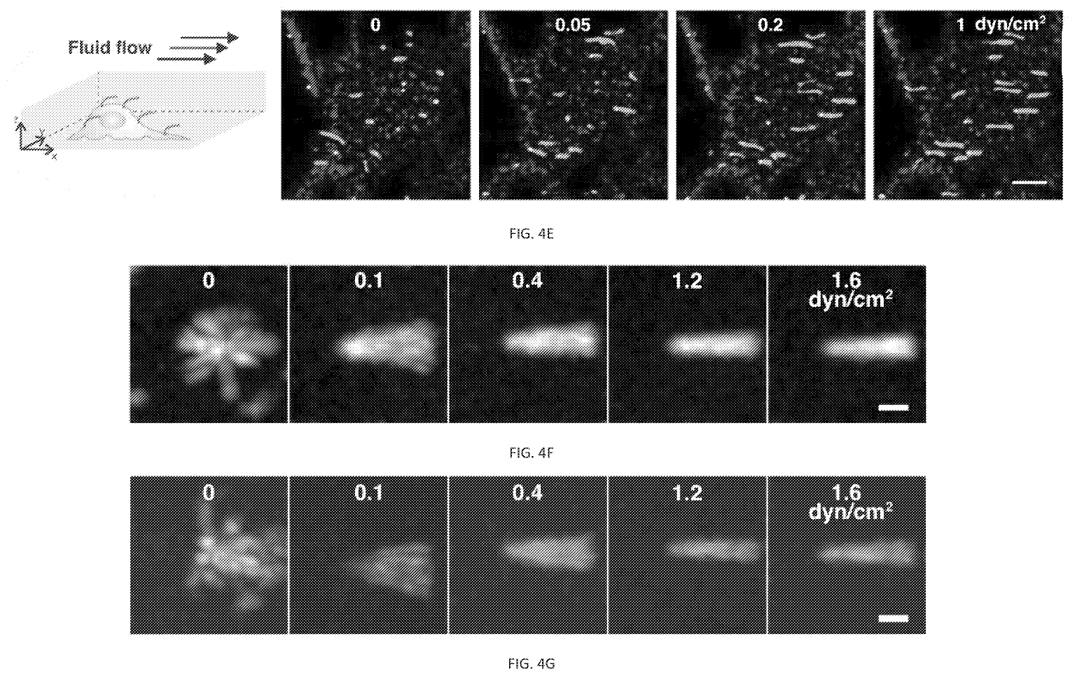

To assess this possibility, a model of how a nanotube anchored to the surface of a rectangular chamber would respond to shear stress induced by laminar flow of velocity U (FIG. 4A) was developed. A nanotube was modeled as a rigid rod anchored by a flexible linker. It was assumed that the chamber was much taller than the nanotube's length so the flow field around the nanotube would be essentially uniform (FIG. 4B). In simulations, the polar angle between the nanotube and z-axis was close to π/2 except at very small shear stresses, so it was further assumed this polar angle was π/2 under external flow. The nanotube's response could therefore be reduced to an in-plane rotation, i.e. the azimuth angle, ϕ between the nanotube and the flow's direction (FIG. 4B). In this case, the flow-induced viscous drag on the nanotube is F=(aμU [symbol] , 0), where a is the coefficient of viscous drag on the nanotube, μ is the viscosity of the fluid in the chamber, and [symbol] is the nanotube's length. The directional vector of the center of mass of the nanotube is r=( [symbol] /2 cos ϕ, [symbol] /2 sin ϕ). Thus, the torque on the nanotube is M=r×F=−½μU [symbol] sin ϕ. This torque was used to calculate the dynamics of the nanotube, which are governed by γdϕ/dt=M+R, where γ is the nanotube's damping coefficient, M=|M|, and R is a random force from thermal fluctuations. R's distribution is given by P(R)∝exp[−R² Δt I(2k_{B}Tγ)], where Δt is the time step used to numerically evolve the equation. For each time step, a random R was drawn. The initial value of the azimuth angle, ϕ₀, of each nanotube was randomly drawn from the uniform distribution [−π,π]. The probability distribution of ϕ was solved by sampling ϕ for a large number of nanotubes for each a set of volumetric flow rates Q=UHW, where H and W are, respectively, the chamber's height and width (FIG. 4C). It was determined that the distributions of azimuthal angles should vary for shear stresses between 0-1.5 dyn/cm², a range relevant for ion channel activation^{32,33}.

To measure the sensitivity and dynamic range of nanotube flow sensors, nanotube seeds were anchored to the bottom of a passivated glass microchannel and their orientations were measured under different flows using time-lapse spinning disk confocal microscopy (FIG. 4D). In the absence of flow, nanotubes explored all azimuthal angles and bent in the z-direction. A shear stress of only 0.05 dyn/cm² caused the nanotubes to remain in plane and align with the flow (FIG. 4D). To quantify the relationship between nanotube orientation and fluid shear stress on glass, the mean total angle of nanotube rotation was measured over 30 frames taken every 5 seconds at each fluid shear stress. A maximum time projection image of each nanotube was generated from these images indicating the nanotube's total angular range of angular motion, Φ (FIGS. 4F-G). The mean angular range of angular motion for different nanotubes experiencing a given shear stress, Φ̅, varied between 0.05-2 dyn/cm² and decreased with increasing shear stress consistent with the model's predictions (FIG. 4H).

Nanotubes attached to living cells via AMDA also increasingly aligned with the flow as shear stress increased (FIG. 4E). Because cells are not flat, a nanotube's location on a cell affected its bend direction and motion. The total angles of rotation of nanotubes on the tops of cells varied most in response to different flow rates.

The shear stress for nanotubes on the tops of HeLa cells, assuming that the shear stress was the same as at the glass boundary, was close to both the values predicted by the model and the values measured on glass for all shear stresses, suggesting how anchored nanotubes can serve as “windsocks” on cells that indicate the flow direction and the magnitude of shear stress the flow induces.

### Example 3

### Growing Nanotubes on Living Cells

A key advantage of using self-assembled biomolecular structures as cell surface microdevices is that they might dynamically grow or reorganize via biomolecular reactions.

Nanotubes can grow via monomer addition but at monomer concentrations where end-on growth is preferred over homogeneous nucleation, growth occurs at <0.2 μm/hr. Because nanotubes persist only a few hours on EGFR, nanotubes were extended via end-to-end joining of pre-assembled nanotubes.

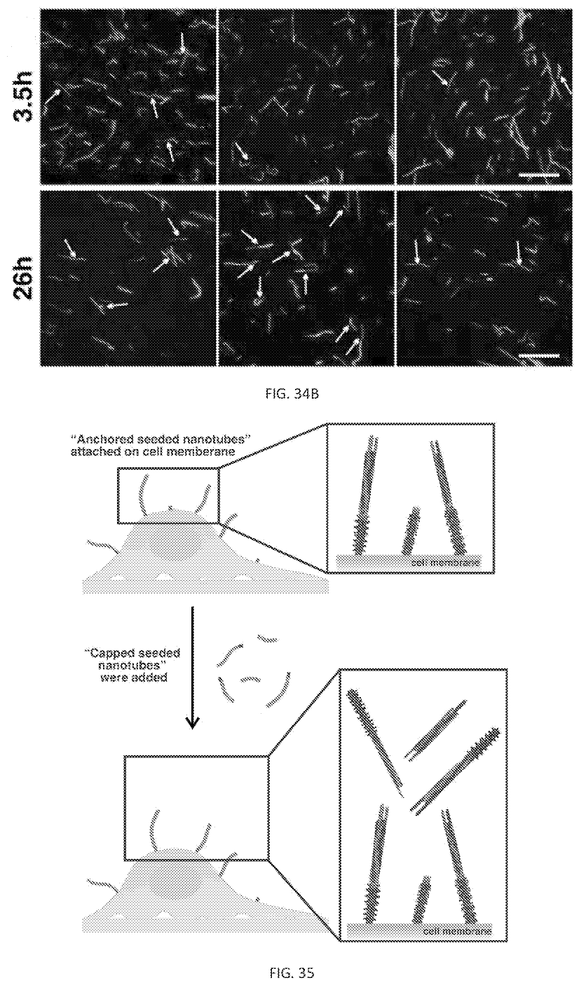

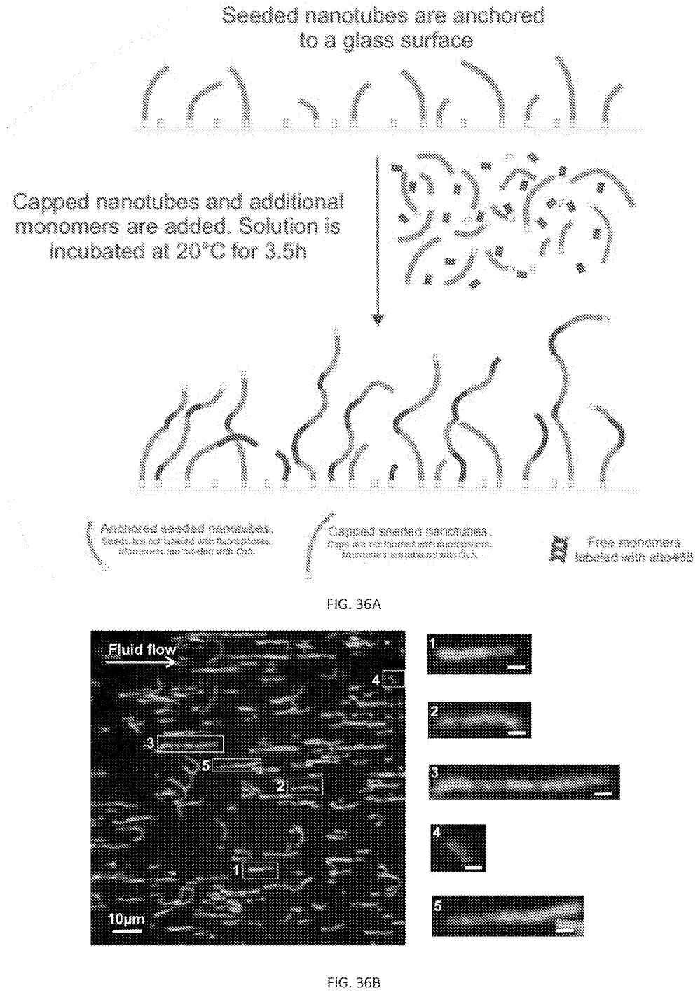

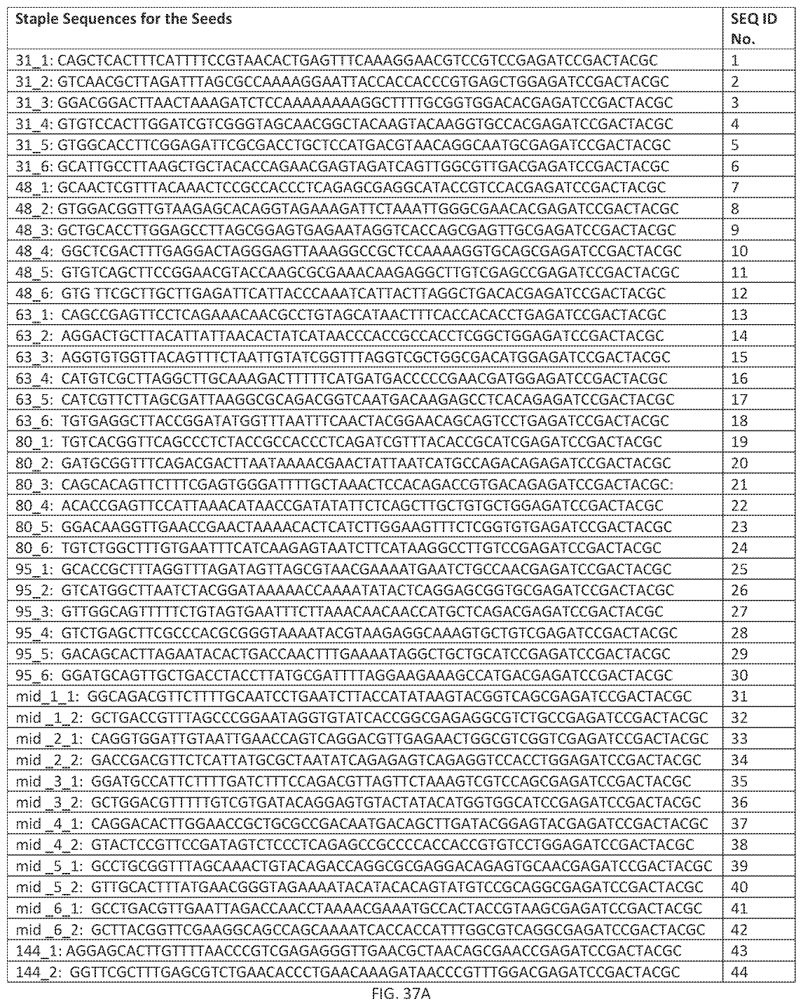

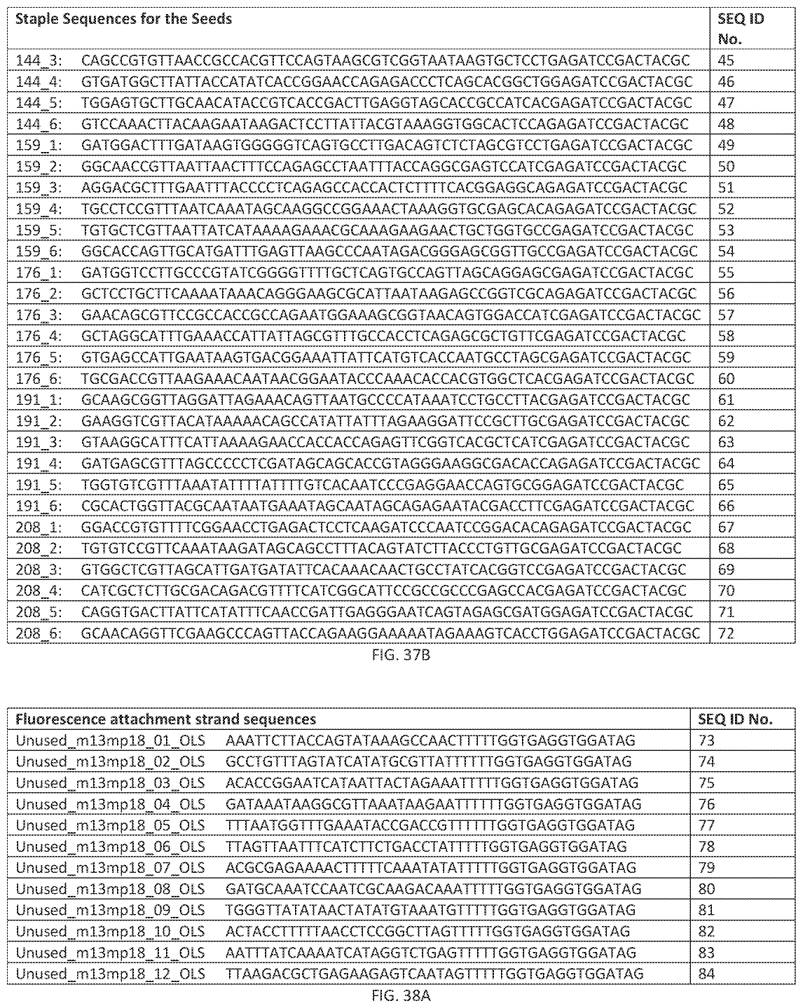

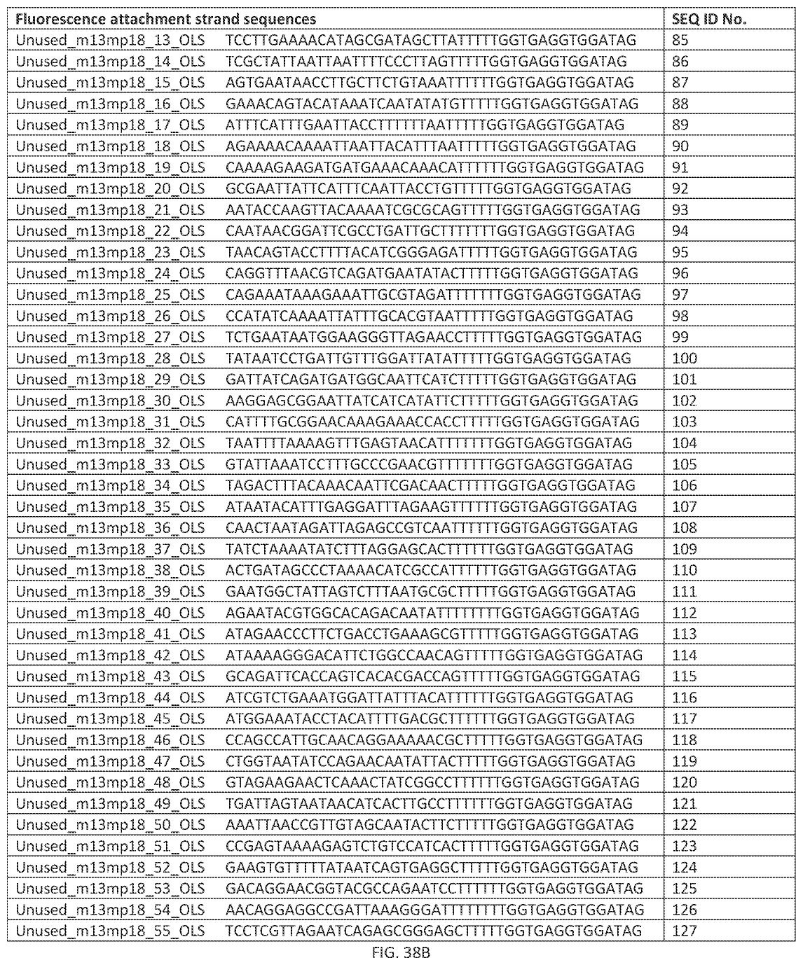

While rapid end-to-end DNA nanotube joining has been observed in vitro, only 7±2% (N=477) of PEG-coated nanotubes underwent end-to-end joining within 4 hours in cell buffer at physiological temperatures (FIG. 34). It was hypothesized that end-to-end joining did not occur because the monomer detachment rate was very low, allowing rough facets or facets with defective monomers that cannot join to persist (FIG. 35). To increase the monomer detachment rate, the monomers' binding sites were shortened from 6 to 4 nucleotides to produce 4PEG nanotubes. 86±3% (N=803) of 4PEG nanotubes anchored to glass surface grew via end-to-end joining within 3.5 hours after 4PEG nanotubes (green) and 150 nM monomers that could serve as “glue” to fill in gaps between rough facets (red) were added (FIG. 36).

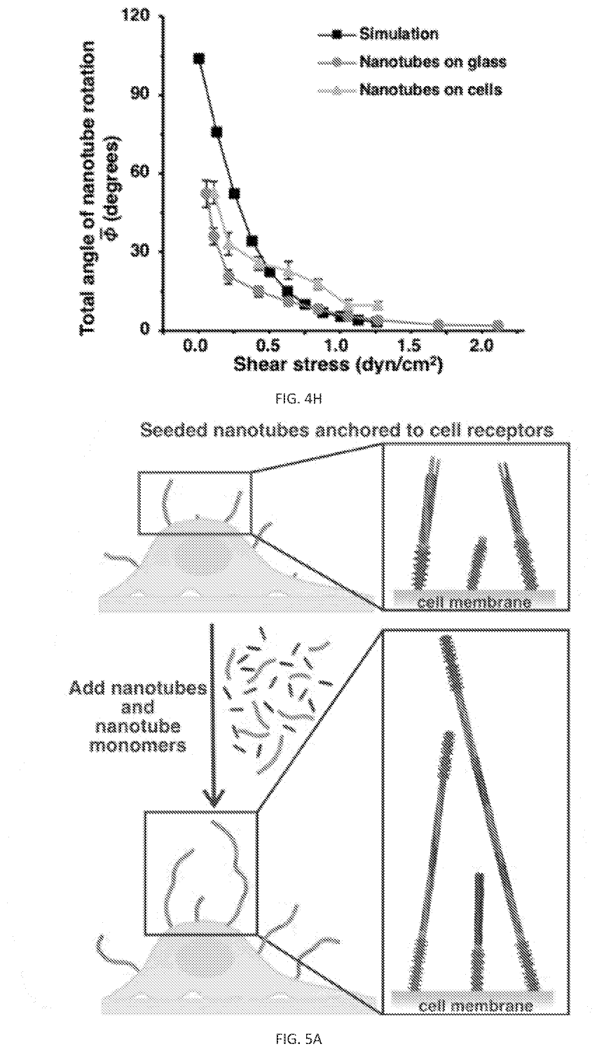

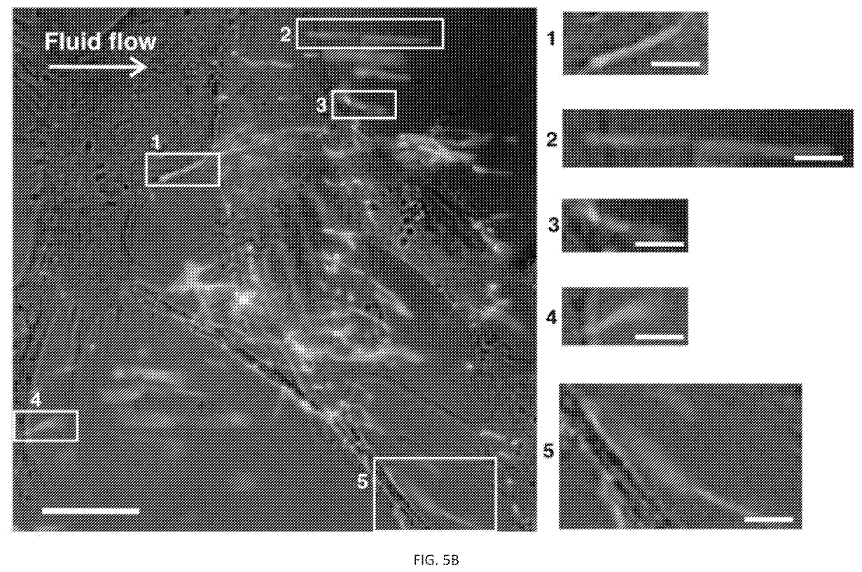

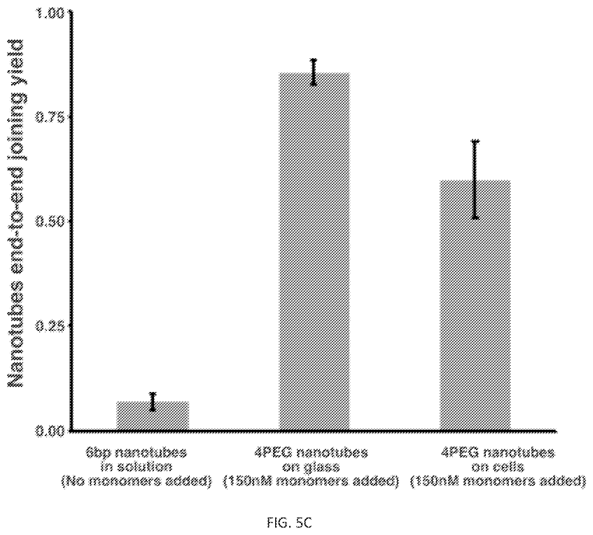

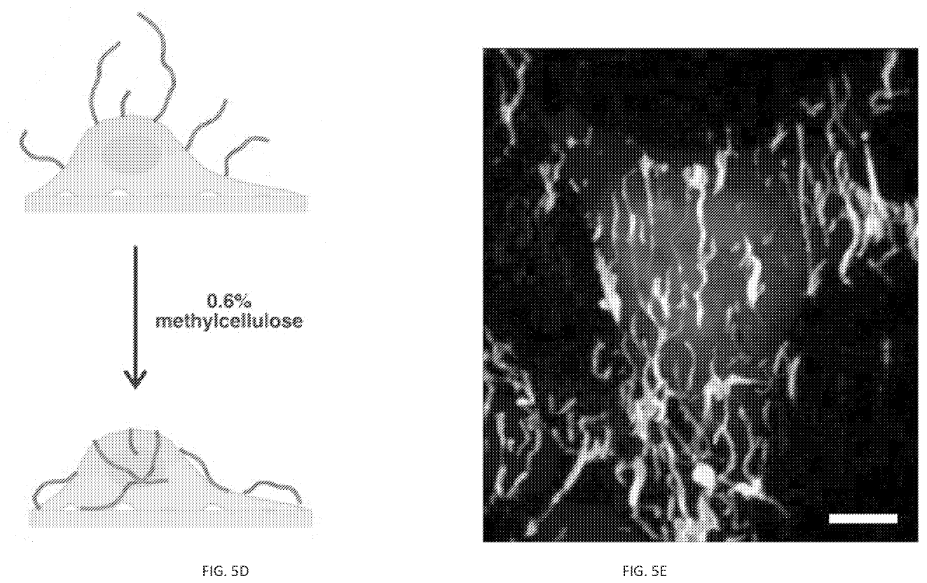

Following this result, 60±9% of 4PEG nanotubes attached to EGFR on HeLa cells were extended using a similar protocol of end-to-end joining and monomer gluing (FIGS. 5A-B). Since the fluid flow used to determine whether individual nanotubes had grown sometimes severed them, the joining process was repeated and methylcellulose was then added to reduce nanotube diffusion. Those processes revealed alternating green-red segments indicating nanotube gluing and joining (FIGS. 5C-E) as well as overlapping red and green segments indicating filament bundling, which high viscosity medium can induce.

While biomolecular filaments are structurally simple, they can assemble in myriad ways to create complex functional materials and devices, as exemplified by cytoskeletal structures. Here synthetic DNA filaments were site-specifically anchored to living cells by determining the required binding affinities, reaction rates and avidity for efficient attachment, and mitigated nonspecific interactions. The resulting precise control over attachment, in combination with the understanding of DNA nanotube growth rates, hierarchical assembly and reorganization, might be used to build a range of synthetic, dynamic filament-based devices on cells, including antennae, motion-inducing devices or connections between different cells.

### Example 4

### Nanotube Structures and Sequences

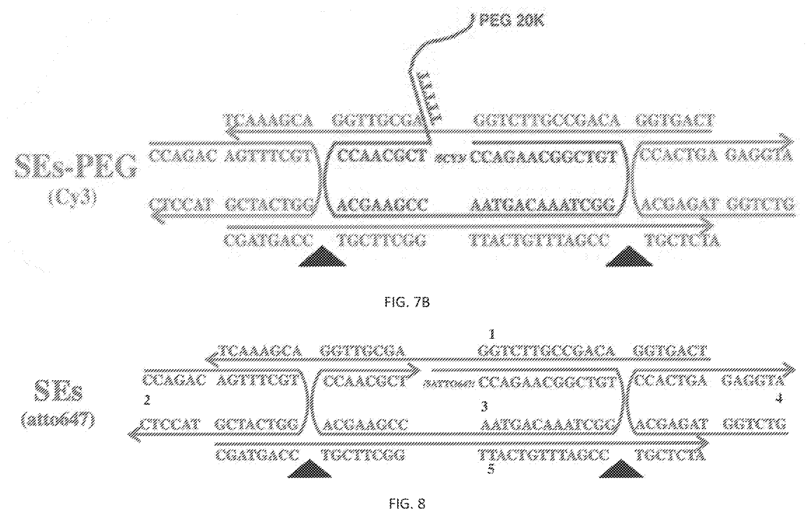

Nanotube monomers with 6 nucleotide sticky end overhangs (6nt monomers) with and without polyethylene glycol (PEG) modification. To ensure stability of the assembled nanotubes, the length of the sticky end overhangs was increased from 5 to 6 nucleotides (FIG. 7A). The Ent nanotube monomer included SEs tile shown in FIG. 7A. The nanotubes assembled from these 6nt monomers grew from seeds at 37° C. To visualize these nanotubes under fluorescence microscope, each SEs tile monomer was labeled with Cy3 by covalently attached the Cy3 fluorophore at the 5′of the central strand SEs _3 (FIG. 7A).

To reduce the nonspecific interactions between seeded nanotubes and the live cell membrane, the nanotube seeds and nanotubes were all modified with polyethylene glycol (PEG). To coat DNA nanotubes with PEG, the central strand of the 6 nt monomer (SEs_3-5′Cy3) was conjugated with PEG via the amine modification (FIG. 7B). In the DNA sequences listed below and in FIGS X-Y, /Cy3/ denotes Cy3 fluorophore covalently attached to the 5′ end of DNA and /3AmMO/ denotes an amino group covalently attached to the 3′ end of DNA.

6nt SEs nanotube monomer sequences:

SEs_1:
(SEQ ID NO: 315)
TCAGTGGACAGCCGTTCTGGAGCGTTGGACGAAACT
SEs_2:
(SEQ ID NO: 316)
CCAGACAGTTTCGTGGTCATCGTACCTC
SEs_3-5′Cy3:
(SEQ ID NO: 317)
/Cy3/CCAGAACGGCTGTGGCTAAACAGTAACCGAA
GCACCAACGCT
SEs_4:
(SEQ ID NO: 318)
GTCTGGTAGAGCACCACTGAGAGGTA
SEs_5:
(SEQ ID NO: 319)
CGATGACCTGCTTCGGTTACTGTTTAGCCTGCTCTA

DNA for PEG modification:

SEs_3-5′Cy3-3′maine:
(SEQ ID NO: 320)
/Cy3/CCAGAACGGCTGTGGCTAAACAGTAACCGA
AGCACCAACGCTTTTTT/3AmMO/

In the nanotube end to end joining experiment, the Ent nanotubes were labeled with Cy3 and atto647 individually. The atto647 fluorophore was covalently attached at the 5′ of the central strand SEs_3. The structure of the atto647 labeled 6nt nanotube monomer is shown in FIG. 8.

The sequence of the central strand of the 6nt monomer labeled with atto647 is as follows:

SEs_3-5′ ATTO 647:
(SEQ ID NO: 321)
/5ATTO647N/CCAGAACGGCTGTGGCTA
AACAGTAACCGAAGCACCAACGCT

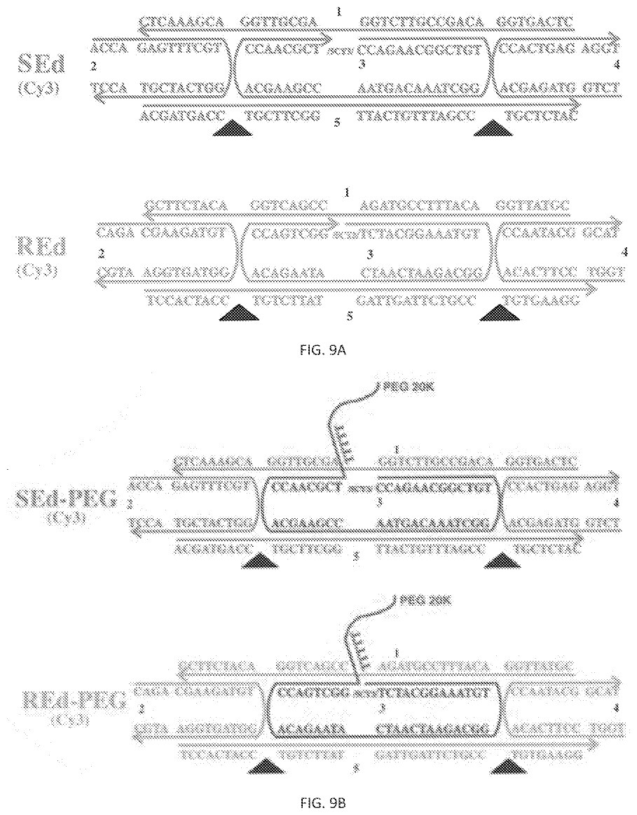

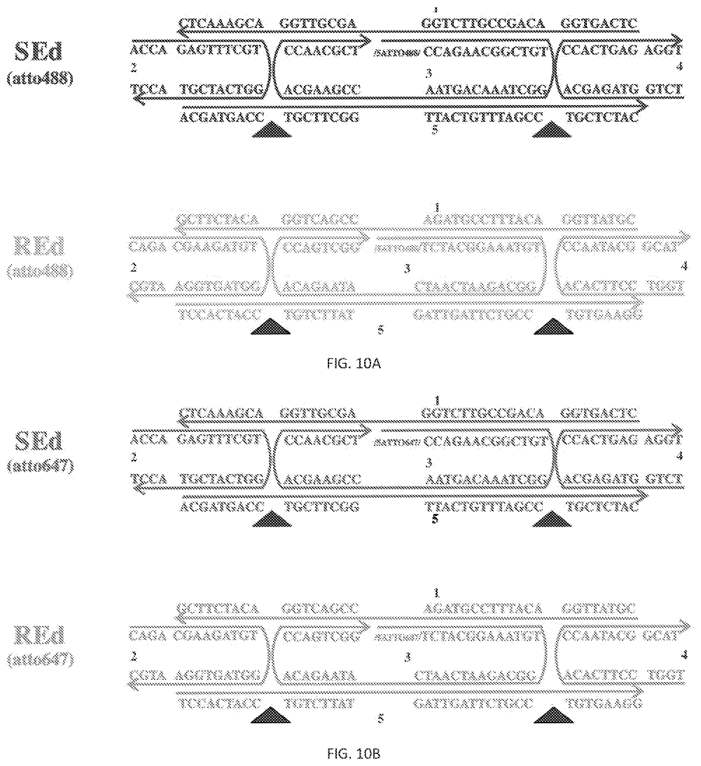

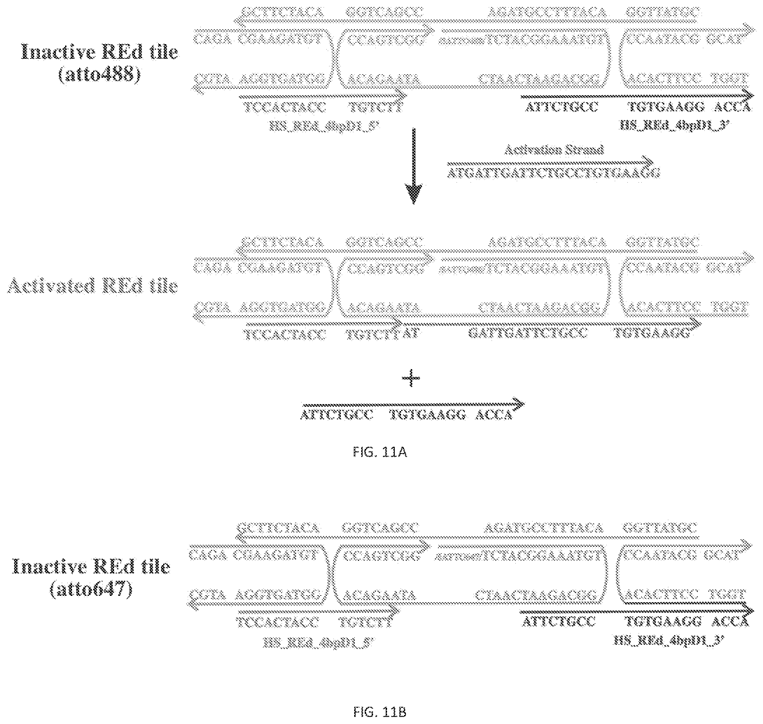

4nt monomers with and without PEG modification The 4nt nanotube monomer included both SEd tile and REd tile shown in FIG. 9A. To coat the 4nt nanotube with PEG, both tiles were modified with PEG by conjugating PEG at the central strands SEd_3-5′Cy3 and REd_3-5′Cy3 as shown in FIG. 9B.

4PEG Cy3 labeled nanotube monomer sequences:

REd_1:
(SEQ ID NO: 322)
CGTATTGGACATTTCCGTAGACCGACTGGACATCTTCG
REd_2:
(SEQ ID NO: 323)
CAGACGAAGATGTGGTAGTGGAATGC
REd_3-5′Cy3:
(SEQ ID NO: 324)
/5Cy3/TCTACGGAAATG TGG CAG AAT CAA TCA
TAA GAC ACC AGT CGG
REd_4:
(SEQ ID NO: 325)
TGGTCCTTCACACCAATACGGCAT
REd_5:
(SEQ ID NO: 326)
TCCACTACCTGTCTTATGATTGATTCTGCCTGTGAAGG
SEd_1:
(SEQ ID NO: 327)
CTCAGTGGACAGCCGTTCTGGAGCGTTGGACGAAACTC
SEd_2:
(SEQ ID NO: 328)
ACCAGAGTTTCGTGGTCATCGTACCT
SEd_3-5′Cy3:
(SEQ ID NO: 329)
/5Cy3/CCAGAACGGCTGTGGCTAAACAGTAACCGAA
GCACCAACGCT
SEd_4:
(SEQ ID NO: 330)
TCTGGTAGAGCACCACTGAGAGGT
SEd_5:
(SEQ ID NO: 331)
ACGATGACCTGCTTCGGTTACTGTTTAGCCTGCTCTAC

Sequences for PEG modified monomers (these strands, after PEG conjugation replace REd_3-5′Cy3 and SEd-3-5′Cy3):

REd_3-5′Cy3-3′amine:
(SEQ ID NO: 332)
/5Cy3/TCTACGGAAATGTGGCAGAATCAA
TCATAAGACACCAGTCGGTTTTT/3AmMO/
SEd_3-5′Cy3-3′amine has the same
sequence as SEs_3-5′Cy3-3′amine.

The 4nt monomers were labeled with atto488 or atto647 by labeling both the central strands SEd_3 and REd_3 as shown in FIG. 10.

The central strand of the 4nt monomer labeled with atto488:

REd_3-5′ATTO488:
(SEQ ID NO: 333)
/5ATTO647N/TCTACGGAAATGTGGCA
GAATCAATCATAAGACACCAGTCGG
SEd_3-5′ ATTO488:
(SEQ ID NO: 334)
/5ATTO647N/CCAGAACGGCTGTGGCT
AAACAGTAACCGAAGCACCAACGCT

The central strand of the 4nt monomer labeled with atto647:

REd_3-5′ATTO647:
(SEQ ID NO: 335)
/5ATTO647N/TCTACGGAAATGTGGCAG
AATCAATCATAAGACACCAGTCGG
SEd 3-5′ ATTO 647:
has the same sequence as SEs_3-5′ATTO 647.

4nt inactive monomer design One of the sticky ends of the 4nt REd monomer (REd5) was blocked and was then exposed, after adding the active strands. In this process, until adding the active strands, the inactive monomer did not polymerize to the nanotube after annealing. Therefore, it is possible to anneal the inactive monomer strand at very high concentration without any unseeded nanotube growth.

These inactive 4nt inactive monomers were also labeled with different fluorophores like atto488 and atto647. The 4nt Atto488-labeled inactive monomer included atto488 labeled inactive REd tile (FIG. 11A) and atto488 labeled normal 4nt SEd tile (FIG. 10A). 4nt Atto647-labeled inactive monomer included atto647 labeled inactive REd tile (FIG. 11B) and atto647 labeled normal 4nt SEd tile (FIG. 10B).

Inactive tile strands:

HS_REd_4bpD1_5′
(SEQ ID NO: 336)
TCCACTACCTGTCTT
HS REd_4bpD1
(SEQ ID NO: 337)
3′ ATTCTGCCTGTGAAGGACCA

Activation strands:

(SEQ ID NO: 338)
Activation strand
ATGATTGATTCTGCCTGTGAAGG

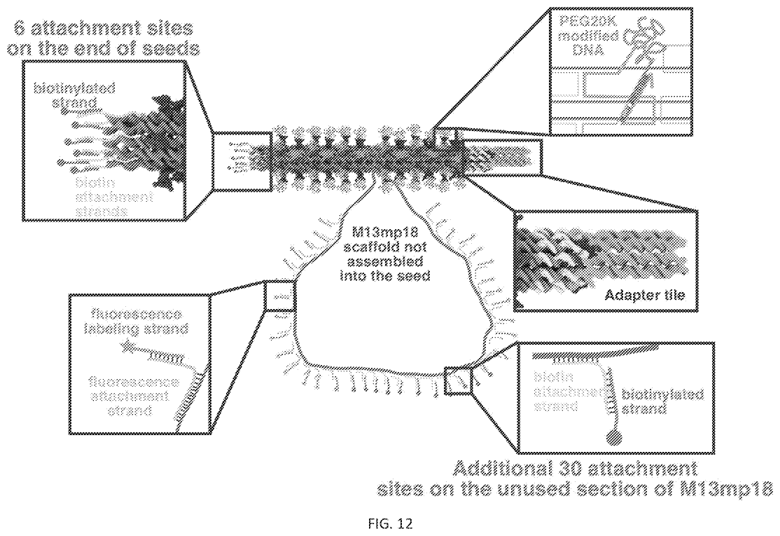

Design and sequences of DNA nanotube seeds The DNA origami seeds serve as nuclei and direct the assembly of DNA nanotubes and function as anchors for DNA nanotubes to attach on specific receptors on cell membranes. The different parts of the DNA origami seeds were designed to realize these functions (FIG. 12).

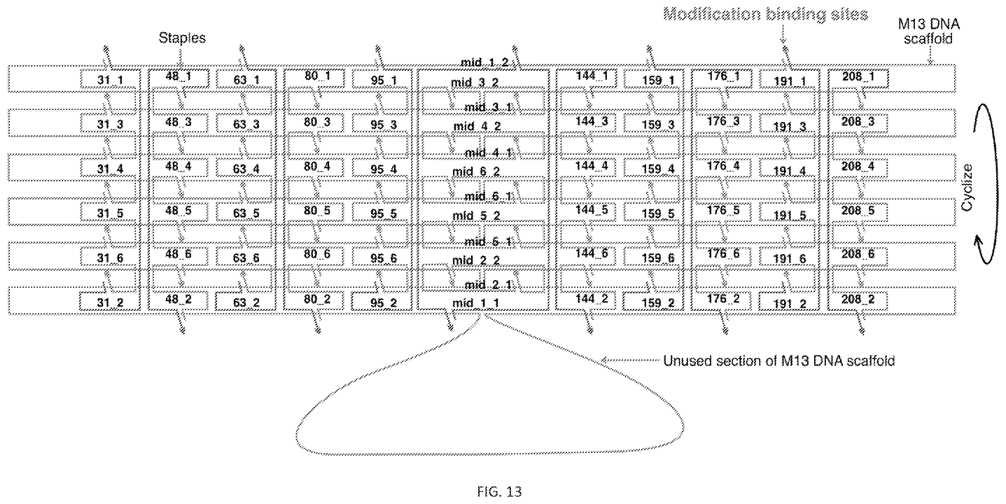

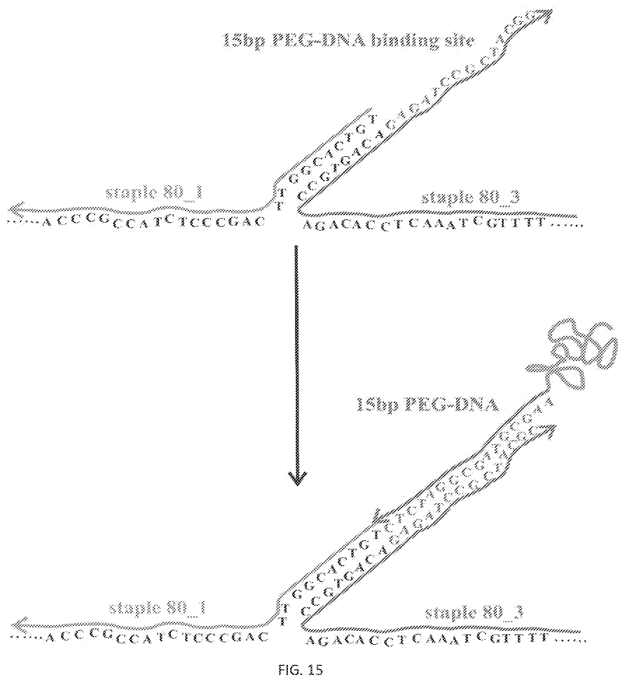

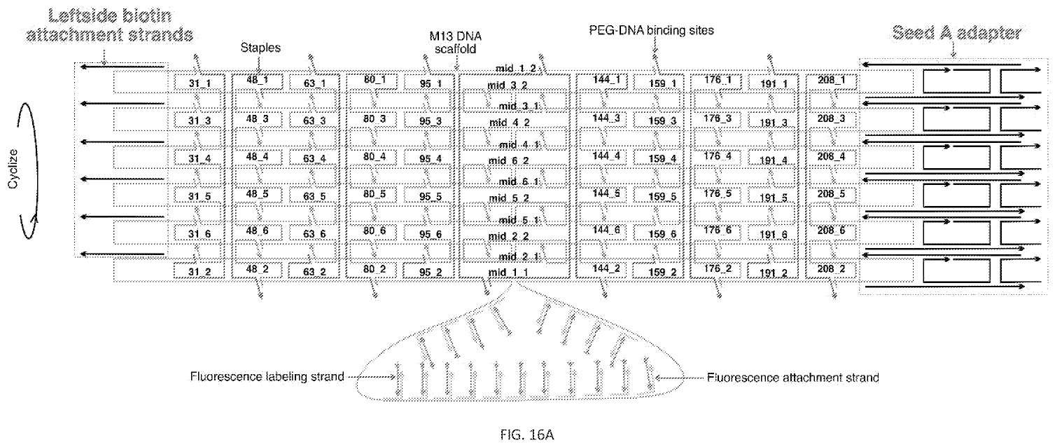

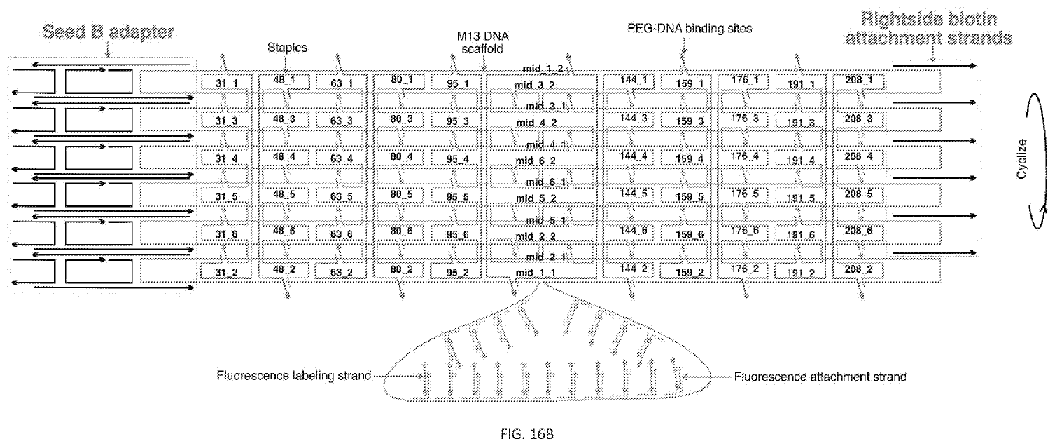

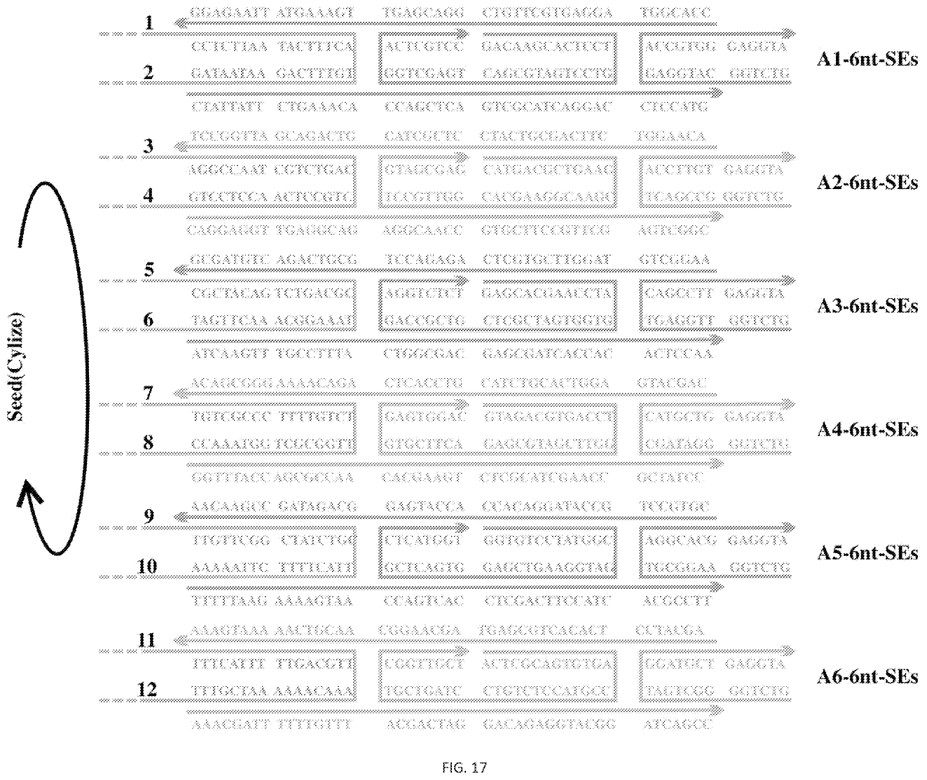

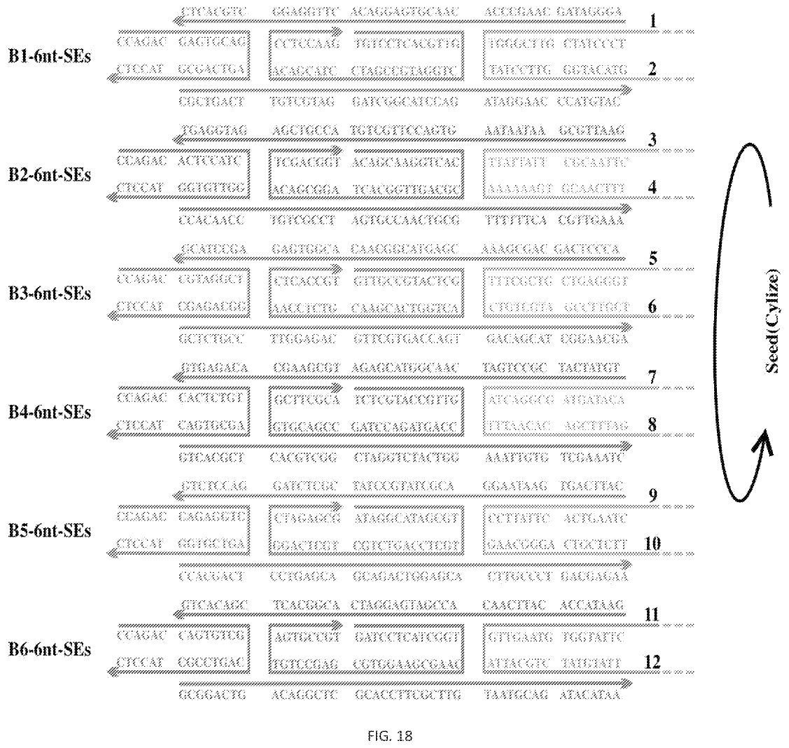

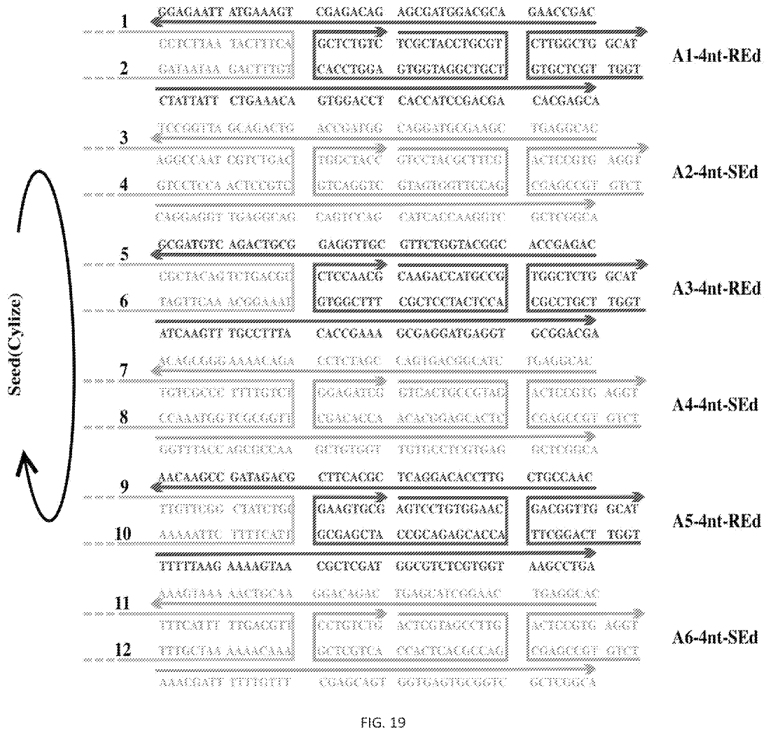

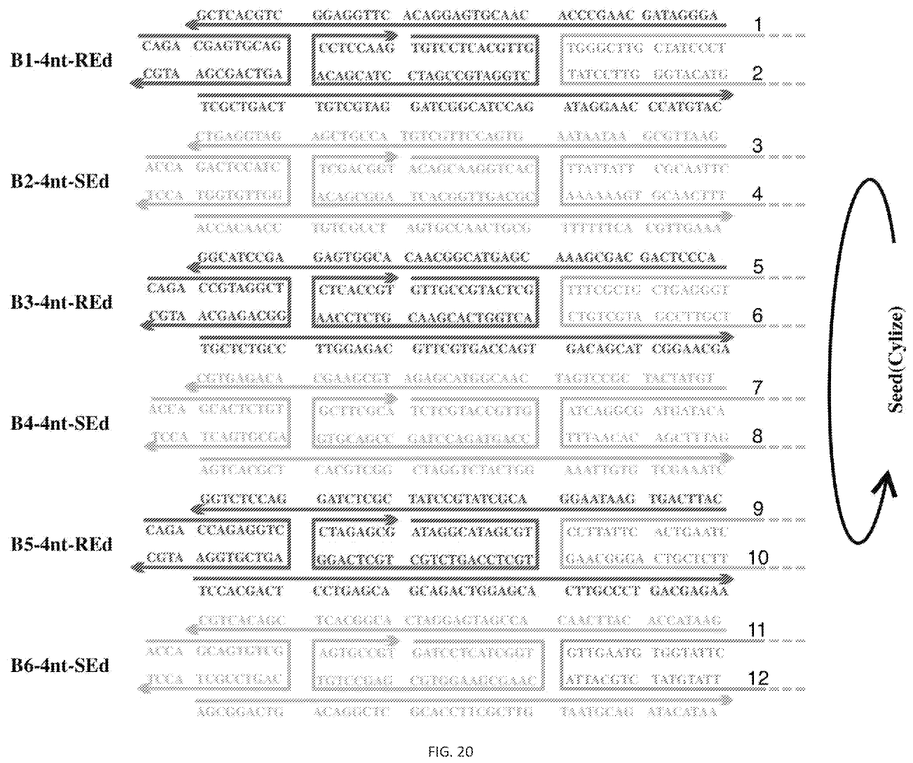

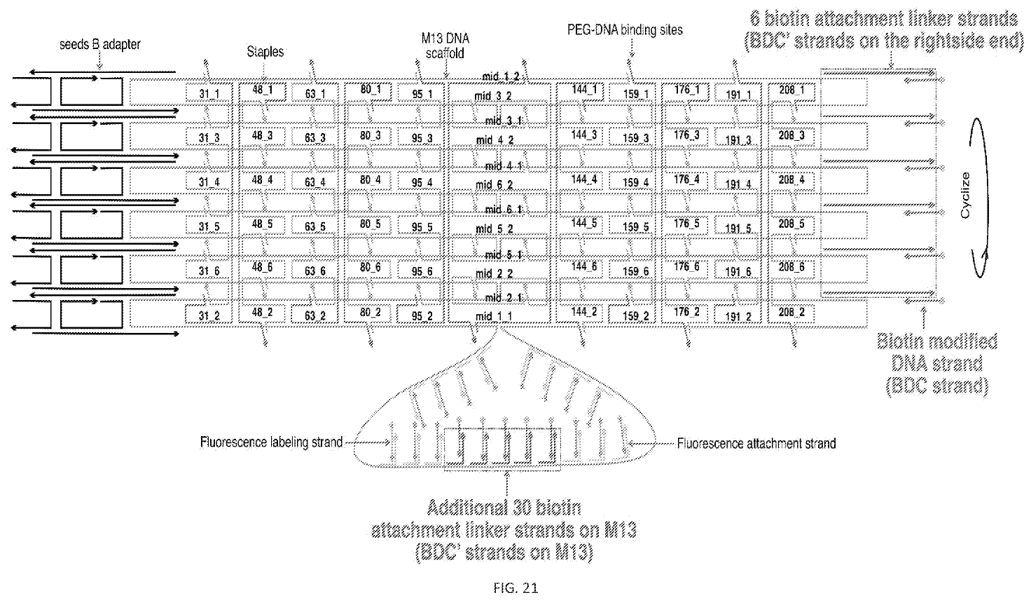

DNA nanotube seeds with PEG coating The DNA nanotube seeds used in this work were adopted from Mohammed et al. (Nano Letters 13, 4006-4013, (2013), incorporated herein by reference). In the original nanotube seed design, the staples presented DNA hairpins at even helical turns so that all the hairpins were on one side of the unrolled seed. Each of these staples was modified by breaking the hairpin into two parts and extending one of the resulting sequences by 15 nucleotides (FIGS. 13 and 15). These extended single DNA strand tags could be used as binding sites for multiple functional group-DNA conjugates (FIG. 13). i.e. a fluorescence, protein, or polymer coating for the seeds.

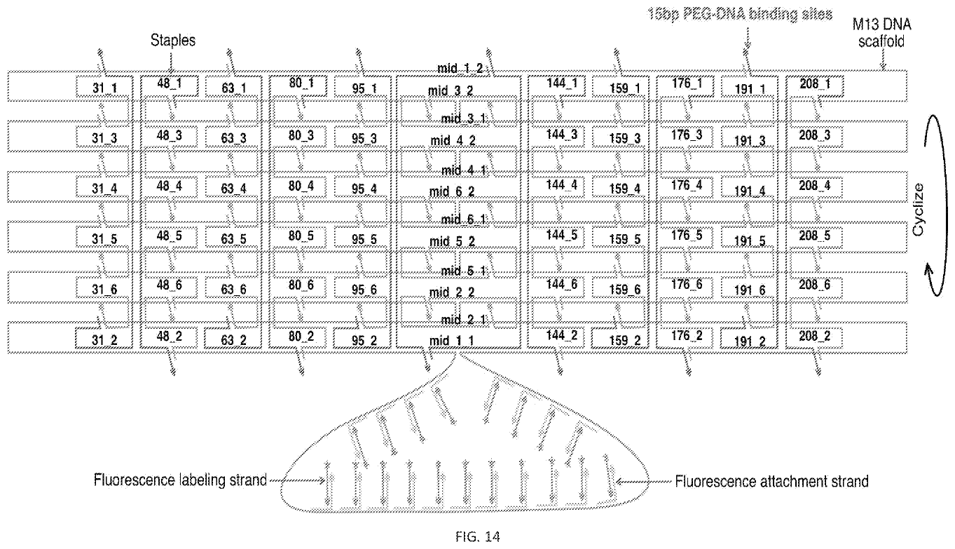

To reduce the nonspecific attachment to the cell membrane, the nanotube seeds were coated with PEG by using a PEG-DNA conjugate. The 15 bp PEG-DNA conjugate will bind to all the 72 binding sites extended from the seeds by DNA hybridization to coat the whole nanotube seeds with PEG (FIGS. 14 and 15).

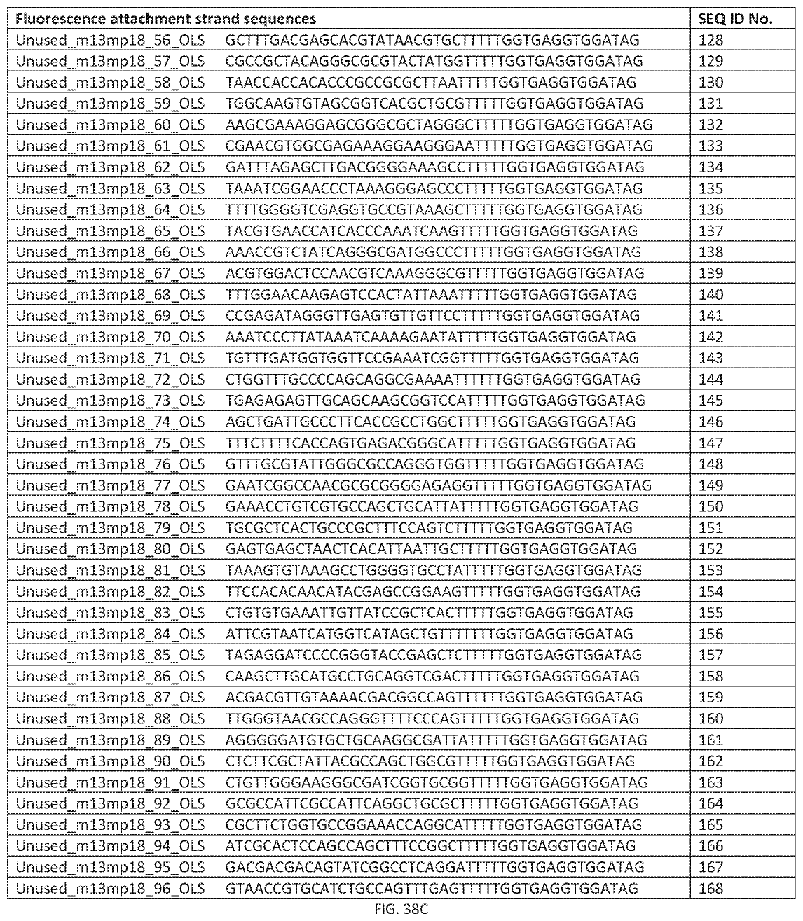

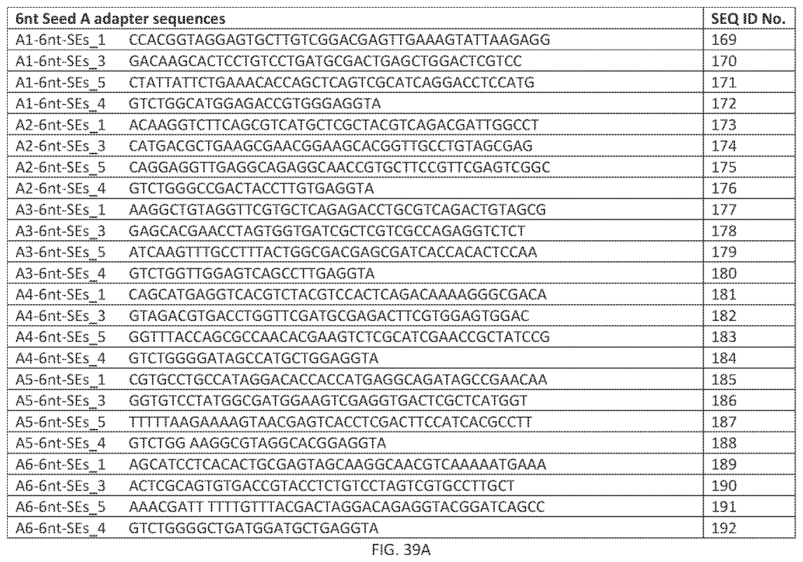

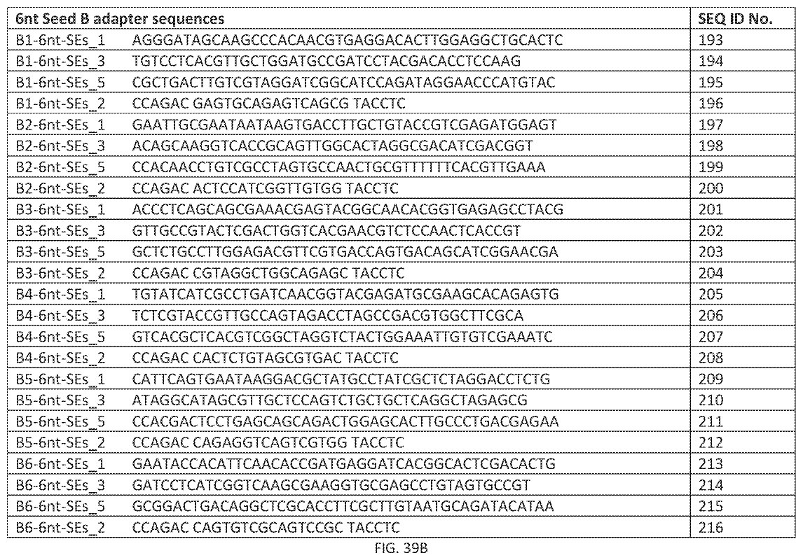

To visualize the seeds using fluorescence and confocal microscopy, the PEG-coated seeds were labelled with fluorophores attached to fluorescent labeling strands that can bind to specific regions of the scaffold. This labeling strategy was adopted from Mohammed et al. (Nano Letters 13, 4006-4013, (2013), incorporated herein by reference), in which 100 fluorescent labeling attachment strands were designed to bind to regions of the M13mp18 scaffold that are not used to fold the seed. Each of these attachment strands includes two domains. One domain binds to the section of the M13mp18 scaffold that is not folded by the staples for the seed, while the other domain binds to a labeling strand with an ATTO fluorophore. Staple sequences for the seeds are shown in FIGS. 37A-37B. Fluorescence attachment strand sequences are shown in FIGS. 38A-38C.

Sequence for the amino-modified DNA strand used to produce a PEG-DNA conjugate that binds to seeds:

Seed PEG-attachment strand
(SEQ ID NO: 339)
/5AmMC6T/AAGCGTAGTCGGATCTC

Fluorescent labeling strand sequences:

Labeling strand ATTO488
(SEQ ID NO: 340)
/5ATTO488N/TTCTATCCACCTCACCA
Labeling strand ATTO647
(SEQ ID NO: 341)
/5ATTO647N/AACTATCCACCTCACCA

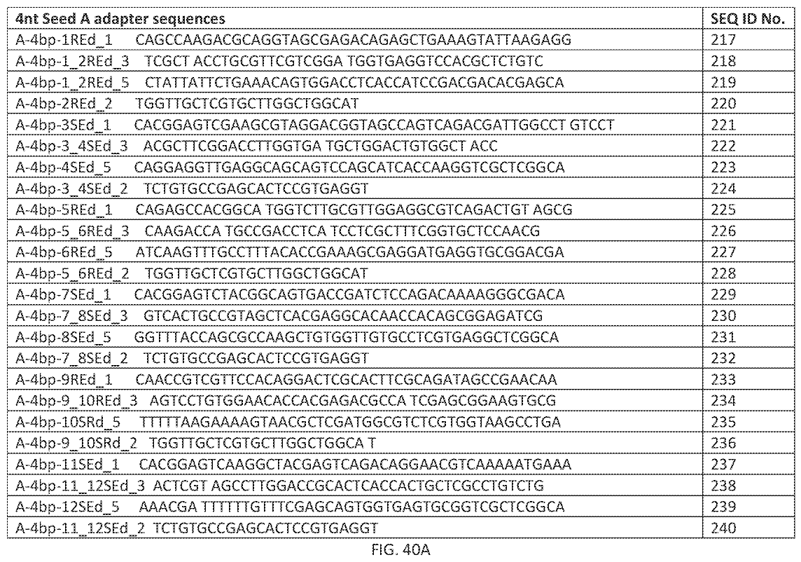

Adapter design and sequences. The seed Ent A adapters served as templates for SEs nanotube growth at the right side of the seeds in FIG. 16A, with corresponding attachment strands for other molecules or structures on the left side of the seeds. The seed Ent B adapters were designed to serve as templates for the SEs nanotube growth from the left side of the seeds as illustrated in FIG. 16B, with attachment strands for other molecules or structures on the right side of the seeds as illustrated. While a variety of attachment chemistries were used on the ends of seeds not used to template growth herein, biotin attachment strands are depicted. Ent Seed A adapter and B adapter sequences are in FIGS. 39A-B.

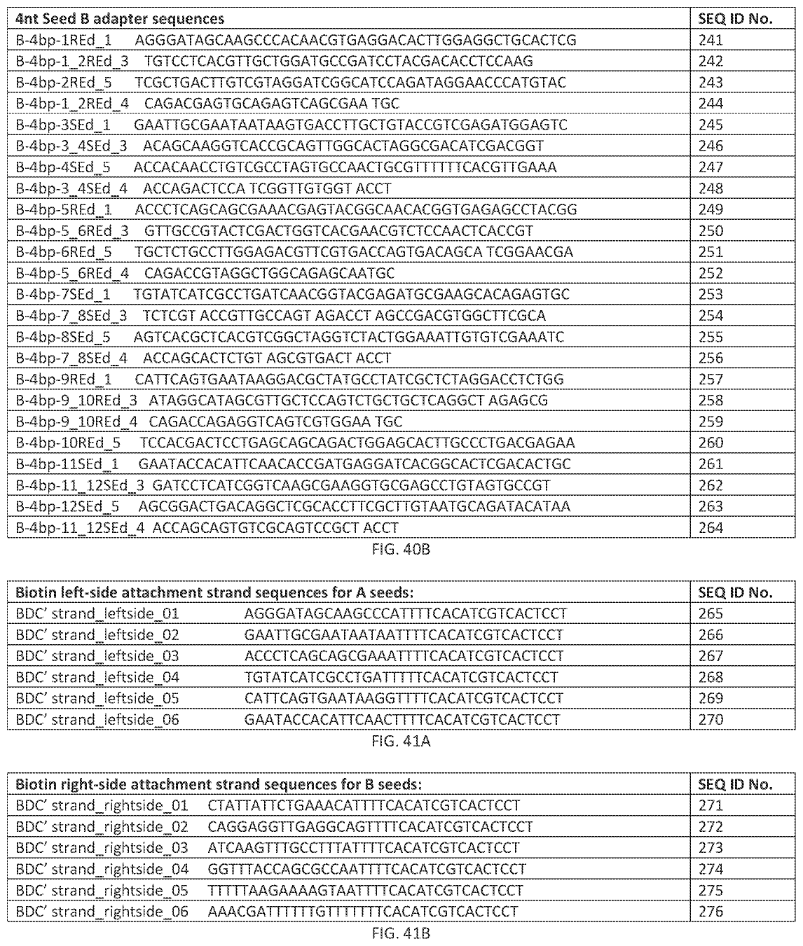

The design of seeds 4nt A adapter and B adapter are listed in FIGS. 40A-B.

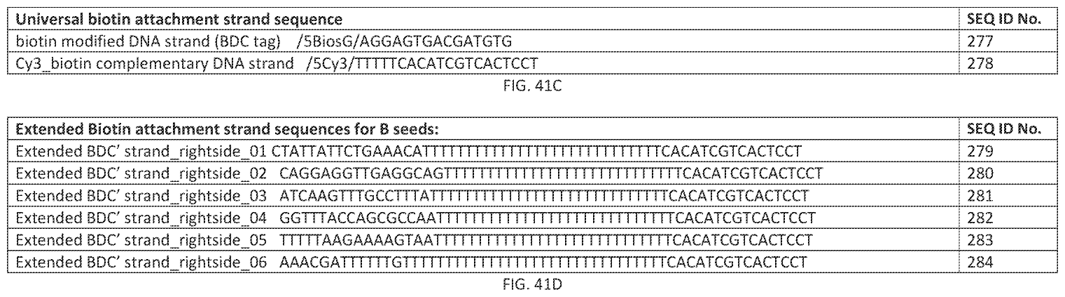

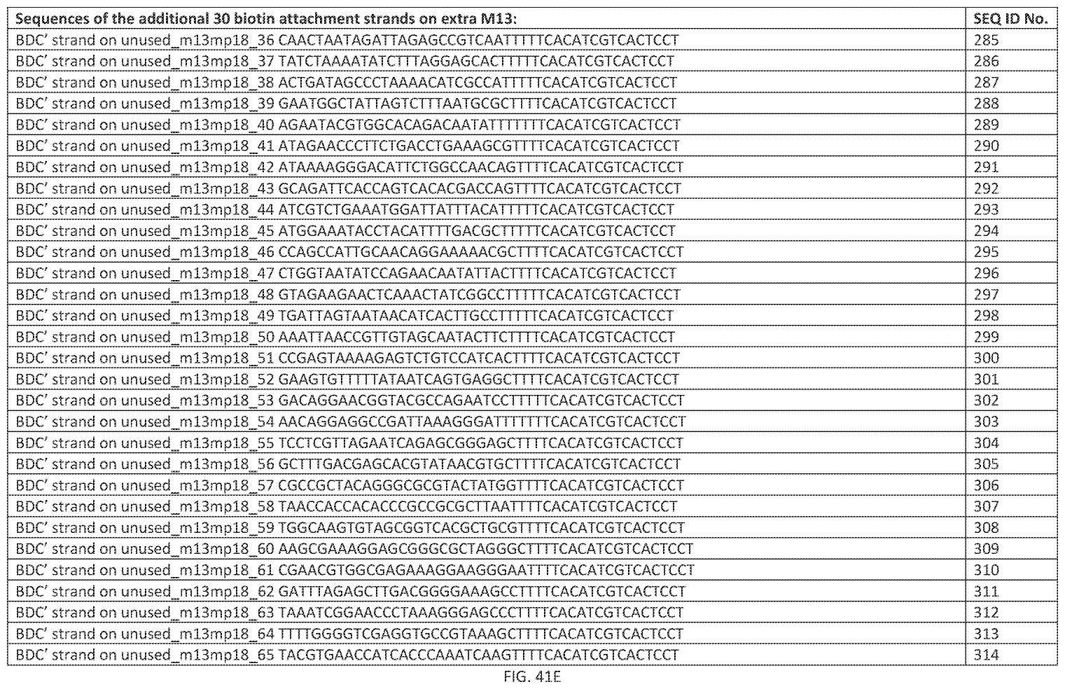

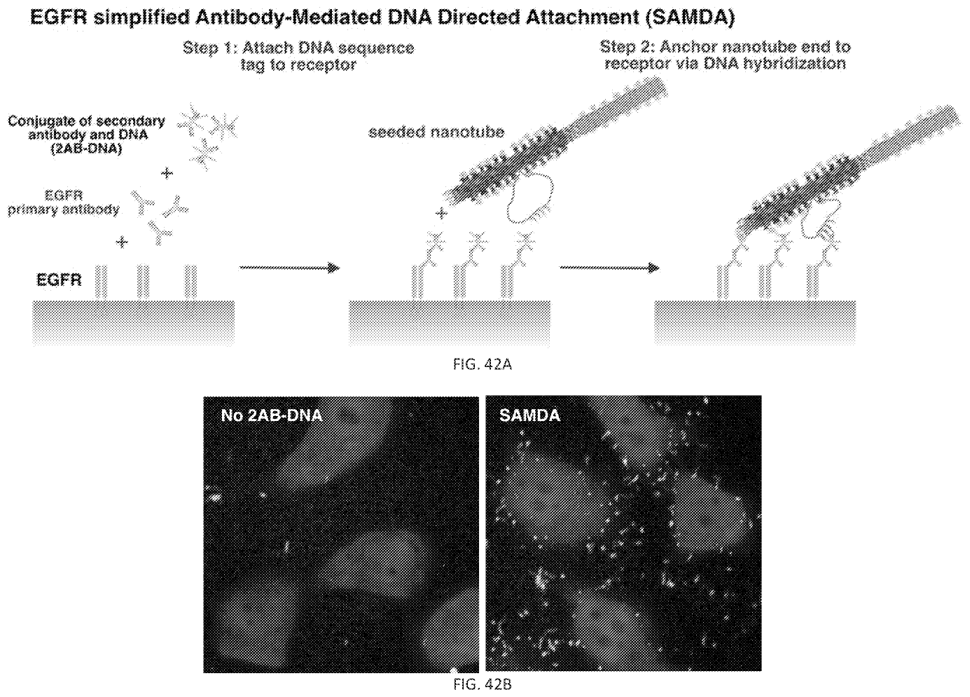

Biotin attachment strand design for AMDA Seeded nanotubes were attached to cells via the hybridization of biotin attachment linker strands presenting BDC′ tags, BDC' strands on seeds to BDC strands presenting the complementary BDC tag on cells. Six BDC′ strands were attached to seeds on their right side, and in most experiments, 30 BDC′ strands were also attached to the center of the unused M13mp18 scaffold (FIG. 21). Biotin attachment strand sequences are in FIGS. 41A-E.

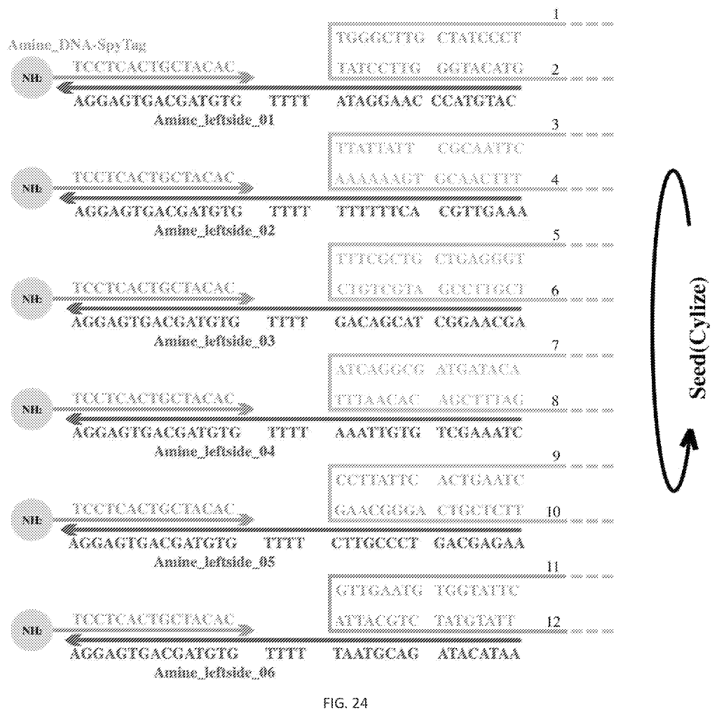

Amino attachment linker strands for SpyTag are listed below and the structure of the amino attachment sites are shown in FIG. 24.

Amino attachment strand sequences:

Amine_leftside_01
(SEQ ID NO: 342)
AGGAGTGACGATGTGTTTTATAGGAACCCATGT AC
Amine_leftside_02
(SEQ ID NO: 343)
AGGAGTGACGATGTGTTTTTTTTTTCACGTTGA AA
Amine leftside_03
(SEQ ID NO: 344)
AGGAGTGACGATGTGTTTTGACAGCATCGGAACGA
Amine leftside_04
(SEQ ID NO: 345)
AGGAGTGACGATGTGTTTTAAATTGTGTCGAAATC
Amine leftside_05
(SEQ ID NO: 346)
AGGAGTGACGATGTGTTTTCTTGCCCTGACGAGAA
Amine leftside_06
(SEQ ID NO: 347)
AGGAGTGACGATGTGTTTTTAATGCAGATACATAA

Amino-DNA Sequence for Conjugation with Spytag or Spycatcher:

Amino_DNA_SpyTag
(SEQ ID NO: 348)
CACATCGTCACTCCT /3AmMO/

### Example 5

### Simplified Antibody-Mediated DNA Directed Attachment (SAMDA)

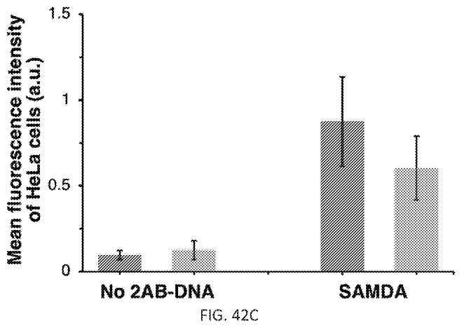

The secondary antibody (2AB) was first conjugated with streptavidin (STA) by a commercial conjugation kit with high yield of secondary antibody-streptavidin conjugates (2AB-STA). Then excess amount of BDC tag was added to this 2AB-STA conjugates solution to form the conjugate of secondary antibody-streptavidin-BDC-A tag (2AB-DNA). Excess BDC-tag was added to block all the binding sits on the streptavidin in the solution. The AMDA method with the use of the secondary antibody and DNA conjugate (2AB-DNA) was termed as simplified AMDA method (FIG. 42A).

Here A seeded nanotubes were specifically anchored to the epidermal growth factor receptor (EGFR) which overexpressed on the HeLa cell membrane by the simplified AMDA method with the use of EGFR primary antibody and the 2AB-DNA conjugation. The PEG coated A seeded nanotube were reliably attached to the cell membrane through this simplified EGFR AMDA but not in controls (FIG. 42B). For nanotube seeds, there were 10-fold more seeds after simplified AMDA vs. the control omitted addition of the conjugate 2AB-DNA. For seeded nanotube, there 5-fold nanotubes on cell than control (FIG. 42C).

### REFERENCES

1. Jennifer Lippincott-Schwartz, Theresa H. Roberts, a. & Hirschberg, K. Secretory Protein Trafficking and Organelle Dynamics in Living Cells. Annual Review of Cell and Developmental Biology 16, 557-589, doi:10.1146/annurev.cellbio.16.1.557 (2000).
2. Shin, Y. & Brangwynne, C. P. Liquid phase condensation in cell physiology and disease. Science 357, eaaf4382, doi:10.1126/science.aaf4382 (2017).
3. Phua, S. C., Lin, Y.-C. & Inoue, T. An intelligent nano-antenna: Primary cilium harnesses TRP channels to decode polymodal stimuli. Cell Calcium 58, 415-422, doi:https://doi.org/10.1016/j.ceca.2015.03.005 (2015).
4. Fletcher, D. A. & Mullins, R. D. Cell mechanics and the cytoskeleton. Nature 463, 485-492, doi:10.1038/nature08908 (2010).
5. Reitsma, S., Slaaf, D. W., Vink, H., van Zandvoort, M. A. M. J. & oude Egbrink, M. G. A. The endothelial glycocalyx: composition, functions, and visualization. Pflugers Archiv—European Journal of Physiology 454, 345-359, doi:10.1007/s00424-007-0212-8 (2007).
6. Elbert, D. L. Bottom-up tissue engineering. Current Opinion in Biotechnology 22, 674-680, doi:https://doi.org/10.1016/j.copbio.2011.04.001 (2011).
7. Bettinger, C. J., Langer, R. & Borenstein, J. T. Engineering Substrate Topography at the Micro- and Nanoscale to Control Cell Function. Angewandte Chemie International Edition 48, 5406-5415, doi:10.1002/anie.200805179 (2009).
8. Feiner, R. & Dvir, T. Tissue—electronics interfaces: from implantable devices to engineered tissues. Nature Reviews Materials 3, 17076, doi:10.1038/natrevmats.2017.76 (2017).
9. Nel, A. E. et al. Understanding biophysicochemical interactions at the nano-bio interface. Nature Materials 8, 543-557, doi:10.1038/nmat2442 (2009).
10. Fattahi, P., Yang, G., Kim, G. & Abidian, M. R. A Review of Organic and Inorganic Biomaterials for Neural Interfaces. Advanced Materials 26, 1846-1885, doi:10.1002/adma.201304496 (2014).
11. Jiang, W., Kim, B. Y. S., Rutka, J. T. & Chan, W. C. W. Nanoparticle-mediated cellular response is size-dependent. Nature Nanotechnology 3, 145, doi:10.1038/nnano.2008.30 https://www.nature.com/articles/nnano.2008.30#supplementary-information (2008).
12. Fan, J., Wang, H.-H., Xie, S., Wang, M. & Nie, Z. Engineering Cell-Surface Receptors with DNA Nanotechnology for Cell Manipulation. ChemBioChem n/a, doi:10.1002/cbic.201900315.
13. Yu, B., Tai, H. C., Xue, W., Lee, L. J. & Lee, R. J. Receptor-targeted nanocarriers for therapeutic delivery to cancer. Mol Membr Biol 27, 286-298, doi:10.3109/09687688.2010.521200 (2010).
14. Dreaden, E. C., Austin, L. A., Mackey, M. A. & El-Sayed, M. A. Size matters: gold nanoparticles in targeted cancer drug delivery. Therapeutic Delivery 3, 457-478, doi:10.4155/tde.12.21 (2012).
15. Habibi, N., Kamaly, N., Memic, A. & Shafiee, H. Self-assembled peptide-based nanostructures: Smart nanomaterials toward targeted drug delivery. Nano Today 11, 41-60, doi:https://doi.org/10.1016/j.nantod.2016.02.004 (2016).
16. Charoenphol, P. & Bermudez, H. Aptamer-Targeted DNA Nanostructures for Therapeutic Delivery. Molecular Pharmaceutics 11, 1721-1725, doi:10.1021/mp500047b (2014).
17. Vogel, V. & Sheetz, M. Local force and geometry sensing regulate cell functions. Nature Reviews Molecular Cell Biology 7, 265-275, doi:10.1038/nrm1890 (2006).
18. Pollard, T. D. & Cooper, J. A. Actin, a Central Player in Cell Shape and Movement. Science 326, 1208-1212, doi:10.1126/science.1175862 (2009).
19. Mohammed, A. M., S̆ulc, P., Zenk, J. & Schulman, R. Self-assembling DNA nanotubes to connect molecular landmarks. Nature Nanotechnology 12, 312, doi:10.1038/nnano.2016.277 https://www.nature.com/articles/nnano.2016.277#supplementary-information (2016).
20. Janmey, P. A. et al. The mechanical properties of actin gels. Elastic modulus and filament motions. Journal of Biological Chemistry 269, 32503-32513 (1994).
21. Rothemund, P. W. K. et al. Design and Characterization of Programmable DNA Nanotubes. Journal of the American Chemical Society 126, 16344-16352, doi:10.1021/ja0443191 (2004).
22. Mohammed, A. M. & Schulman, R. Directing Self-Assembly of DNA Nanotubes Using Programmable Seeds. Nano Letters 13, 4006-4013, doi:10.1021/n1400881w (2013).
23. Axel Ekani-Nkodo, A. K., Deborah Kuchnir Fygenson. Joining and Scission in the Self-Assembly of Nanotubes from DNA Tiles. Physical Review Letters 93, 268301, doi:10.1103/PhysRevLett.93.268301 (2004).
24. Schulman, R. & Winfree, E. Synthesis of crystals with a programmable kinetic barrier to nucleation. Proceedings of the National Academy of Sciences 104, 15236-15241, doi:10.1073/pnas.0701467104 (2007).
25. Barish, R. D., Schulman, R., Rothemund, P. W. K. & Winfree, E. An information-bearing seed for nucleating algorithmic self-assembly. Proceedings of the National Academy of Sciences 106, 6054-6059, doi:10.1073/pnas.0808736106 (2009).
26. Agrawal, D. K. et al. Terminating DNA Tile Assembly with Nanostructured Caps. ACS Nano 11, 9770-9779, doi:10.1021/acsnano.7b02256 (2017).
27. Samuel W Schaffter, L. N. G., Joanna Schneider, Hari K K Subramanian, Rebecca Schulman, Elisa Franco. T7 RNA polymerase non-specifically transcribes and induces disassembly of DNA nanostructures. Nucleic Acids Research 46, 5332-5343, doi:10.1093/nar/gky283 (2018).
28. Jorgenson, T. D., Mohammed, A. M., Agrawal, D. K. & Schulman, R. Self-Assembly of Hierarchical DNA Nanotube Architectures with Well-Defined Geometries. ACS Nano 11, 1927-1936, doi:10.1021/acsnano.6b08008 (2017).
29. Sharma, J. et al. Control of Self-Assembly of DNA Tubules Through Integration of Gold Nanoparticles. Science 323, 112-116, doi:10.1126/science.1165831 (2009).
30. Hariadi, R. F. et al. Mechanical coordination in motor ensembles revealed using engineered artificial myosin filaments. Nature Nanotechnology 10, 696, doi:10.1038/nnano.2015.132 https://www.nature.com/articles/nnano.2015.132#supplementary-information (2015).
31. Stephanopoulos, N. et al. Bioactive DNA-Peptide Nanotubes Enhance the Differentiation of Neural Stem Cells Into Neurons. Nano Letters 15, 603-609, doi:10.1021/n1504079q (2015).
32. Davies, P. F. Flow-mediated endothelial mechanotransduction. Physiological Reviews 75, 519-560, doi:10.1152/physrev.1995.75.3.519 (1995).
33. Su, S. et al. Genetically encoded calcium indicator illuminates calcium dynamics in primary cilia. Nature Methods 10, 1105, doi:10.1038/nmeth.2647 https://www.nature.com/articles/nmeth.2647#supplementary-information (2013).
34. Landry, J. P., Ke, Y., Yu, G.-L. & Zhu, X. D. Measuring affinity constants of 1450 monoclonal antibodies to peptide targets with a microarray-based label-free assay platform. J Immunol Methods 417, 86-96, doi:10.1016/j.jim.2014.12.011 (2015).
35. MacGurn, J. A., Hsu, P.-C. & Emr, S. D. Ubiquitin and Membrane Protein Turnover: From Cradle to Grave. Annual Review of Biochemistry 81, 231-259, doi:10.1146/annurev-biochem-060210-093619 (2012).
36. Singh, R. & Lillard, J. W. Nanoparticle-based targeted drug delivery. Experimental and Molecular Pathology 86, 215-223, doi:https://doi.org/10.1016/j.yexmp.2008.12.004 (2009).
37. Cedervall, T. et al. Understanding the nanoparticle—protein corona using methods to quantify exchange rates and affinities of proteins for nanoparticles. Proceedings of the National Academy of Sciences 104, 2050-2055, doi:10.1073/pnas.0608582104 (2007).
38. Koyfman, A. Y., Braun, G. B. & Reich, N. O. Cell-Targeted Self-Assembled DNA Nanostructures. Journal of the American Chemical Society 131, 14237-14239, doi:10.1021/ja9015638 (2009).
39. Verma, A. & Stellacci, F. Effect of Surface Properties on Nanoparticle—Cell Interactions. Small 6, 12-21, doi:10.1002/sm11.200901158 (2010).
40. Zakeri, B. et al. Peptide tag forming a rapid covalent bond to a protein, through engineering a bacterial adhesin. Proceedings of the National Academy of Sciences 109, E690-E697, doi:10.1073/pnas.1115485109 (2012).
41. Benedetto, S. et al. Quantification of the expression level of integrin receptor αvβ3 in cell lines and MR imaging with antibody-coated iron oxide particles. Magnetic Resonance in Medicine 56, 711-716, doi:10.1002/mrm.21023 (2006).
42. Bremer, E. et al. Simultaneous Inhibition of Epidermal Growth Factor Receptor (EGFR) Signaling and Enhanced Activation of Tumor Necrosis Factor-related Apoptosis-inducing Ligand (TRAIL) Receptor-mediated Apoptosis Induction by an scFv:sTRAIL Fusion Protein with Specificity for Human EGFR. Journal of Biological Chemistry 280, 10025-10033, doi:10.1074/jbc.M413673200 (2005).
43. Zhou, Y. et al. Impact of Intrinsic Affinity on Functional Binding and Biological Activity of EGFR Antibodies. Molecular Cancer Therapeutics 11, 1467-1476, doi:10.1158/1535-7163.Mct-11-1038 (2012).
44. Schreiber, G., Haran, G. & Zhou, H. X. Fundamental aspects of protein-protein association kinetics. Chem Rev 109, 839-860, doi:10.1021/cr800373w (2009).
45. Wang, P. et al. Visualization of the Cellular Uptake and Trafficking of DNA Origami Nanostructures in Cancer Cells. Journal of the American Chemical Society 140, 2478-2484, doi:10.1021/jacs.7b09024 (2018).
46. Vieira, A. V., Lamaze, C. & Schmid, S. L. Control of EGF Receptor Signaling by Clathrin-Mediated Endocytosis. Science 274, 2086-2089, doi:10.1126/science.274.5295.2086 (1996).
47. Zhang, S., Li, J., Lykotrafitis, G., Bao, G. & Suresh, S. Size-Dependent Endocytosis of Nanoparticles. Advanced Materials 21, 419-424, doi:10.1002/adma.200801393 (2009).
48. Mickler, F. M. et al. Tuning Nanoparticle Uptake: Live-Cell Imaging Reveals Two Distinct Endocytosis Mechanisms Mediated by Natural and Artificial EGFR Targeting Ligand. Nano Letters 12, 3417-3423, doi:10.1021/n1300395q (2012).
49. Sorkin, A. & Duex, J. E. Quantitative analysis of endocytosis and turnover of epidermal growth factor (EGF) and EGF receptor. Curr Protoc Cell Biol Chapter 15, Unit-15.14, doi:10.1002/0471143030.cb1514s46 (2010).
50. Rosivall, L., Mirzahosseini, S., Toma, I., Sipos, A. & Peti-Peterdi, J. Fluid flow in the juxtaglomerular interstitium visualized in vivo. American Journal of Physiology-Renal Physiology 291, F1241-F1247, doi:10.1152/ajprenal.00203.2006 (2006).
51. Rydholm, S. et al. Mechanical properties of primary cilia regulate the response to fluid flow. American Journal of Physiology-Renal Physiology 298, F1096-F1102, doi:10.1152/ajprenal.00657.2009 (2010).
52. Young, Y. N., Downs, M. & Jacobs, C. R. Dynamics of the Primary Cilium in Shear Flow. Biophysical Journal 103, 629-639, doi:https://doi.org/10.1016/j.bpj.2012.07.009 (2012).
53. Su, S. et al. Genetically encoded calcium indicator illuminates calcium dynamics in primary cilia. Nature Methods 10, 1105-1107, doi:10.1038/nmeth.2647 (2013).
54. Nauli, S. M. et al. Polycystins 1 and 2 mediate mechanosensation in the primary cilium of kidney cells. Nature Genetics 33, 129, doi:10.1038/ng1076 (2003).
55. Li, Q. et al. Polycystin-2 Cation Channel Function Is under the Control of Microtubular Structures in Primary Cilia of Renal Epithelial Cells. Journal of Biological Chemistry 281, 37566-37575, doi:10.1074/jbc.M603643200 (2006).
56. Hariadi, R. F., Yurke, B. & Winfree, E. Thermodynamics and kinetics of DNA nanotube polymerization from single-filament measurements. Chem Sci 6, 2252-2267, doi:10.1039/c3sc53331j (2015).
57. Green, L. N. et al. Autonomous dynamic control of DNA nanostructure self-assembly. Nature Chemistry 11, 510-520, doi:10.1038/s41557-019-0251-8 (2019).
58. Pacella, M. S. et al. Characterizing the length-dependence of DNA nanotube end-to-end joining rates.Molecular Systems Design & Engineering, doi:10.1039/C9ME00068B (2020).
59. Popp, D., Yamamoto, A., Iwasa, M. & Maeda, Y. Direct visualization of actin nematic network formation and dynamics. Biochemical and Biophysical Research Communications 351, 348-353, doi:https://doi.org/10.1016/j.bbrc.2006.10.041 (2006).
60. Wilkinson, M. H. F. & Schut, F. Digital Image Analysis of Microbes: Imaging, Morphometry, Fluorometry and Motility Techniques and Applications. (Wiley, 1998).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A composition comprising:
a cell; and
a nucleic acid nanotube, wherein a proximal end of the nanotube is attached to a linker configured to bind a moiety located on the surface of the cell.

2. The composition of claim 1, wherein the cell comprises a mammalian cell.

3. The composition of claim 1 or 2, wherein the nucleic acid nanotube is a DNA nanotube.

4. The composition of any of claims 1-3, wherein the nanotube comprises a plurality of polyethylene glycol (PEG) molecules.

5. The composition of claim 4, wherein the PEG molecule has a molecular weight of approximately 20 kDa.

6. The composition of any of claims 1-5, wherein the nanotube further comprises a fluorescent label.

7. The composition of any of claims 1-6, wherein the linker comprises a pair of antibodies comprising a primary antibody configured to bind a moiety located on the surface of the cell and a corresponding secondary antibody.

8. The composition of claim 7, wherein the secondary antibody is streptavidin conjugated.

9. The composition of claim 7, wherein the secondary antibody is biotinylated and the linker further comprises streptavidin bound to the biotinylated secondary antibody.

10. The composition of claim 8 or 9, wherein the linker further comprises a biotinylated single-stranded polynucleotide which binds the streptavidin.

11. The composition of claim 10, wherein the biotinylated polynucleotide is complementary to at least a portion of a single-stranded polynucleotide of the nanotube.

12. The composition of claim 11, wherein the single-stranded polynucleotide of the nanotube is located on the proximal end of the nanotube or exterior to the nanotube.

13. The composition of any of claims 1-12, wherein the moiety is a cell surface protein.

14. The composition of any of claims 1-13, wherein the moiety is a membrane receptor protein, a cell adhesion protein, or a transporter protein.

15. The composition of any of claims 1-14, wherein the DNA nanotube further comprises an object linked to a distal end.

16. The composition of claim 15, wherein the object is a drug, a cell, or a surface of a device or container.

17. A device comprising the composition of any of claims 1-16.

18. A method for preparing a conjugate of a cell and a nucleic acid nanotube, comprising
a) incubating a cell with a primary antibody configured to bind a moiety located on the surface of the cell;
b) incubating the cell with bound primary antibody with a secondary antibody, wherein the secondary antibody comprises streptavidin;
c) incubating a composition resulting from step b) with a biotinylated polynucleotide complementary to at least a portion of a single-stranded polynucleotide of the nanotube or at least a portion of a single-stranded polynucleotide of a nanotube seed; and
d) incubating a composition resulting from step c) with a composition comprising the nanotube or a nanotube seed.

19. A method for preparing a conjugate of a cell and a nucleic acid nanotube, comprising
a) incubating a cell with a primary antibody configured to bind a moiety located on the surface of the cell;
b) incubating the cell with bound primary antibody with a secondary antibody composition, wherein the second antibody is biotinylated;
c) incubating a composition resulting from step b) with streptavidin or neutravidin;
d) incubating a composition resulting from step c) with a biotinylated polynucleotide complementary to at least a portion of a single-stranded polynucleotide of the nanotube or at least a portion of a single-stranded polynucleotide of a nanotube seed; and
e) incubating a composition resulting from step d) with a composition comprising the nanotube or a nanotube seed.

20. A method for preparing a conjugate of a cell and a nucleic acid nanotube, comprising
a) incubating a cell with a primary antibody configured to bind a moiety located on the surface of the cell;
b) incubating the cell with a secondary antibody conjugate, wherein the secondary antibody conjugate comprises:
i. a biotinylated secondary antibody;
ii. streptavidin or neutravidin;
iii. a biotinylated polynucleotide complementary to at least a portion of a single-stranded polynucleotide of the nanotube or at least a portion of a single-stranded polynucleotide of a nanotube seed; and
e) incubating a composition resulting from step b) with a composition comprising the nanotube or a nanotube seed.

21. The method of claim 20, wherein the secondary antibody conjugate is prepared by a method comprising incubating a biotinylated secondary antibody either simultaneously or sequentially with streptavidin or neutravidin and the biotinylated polynucleotide.

22. The method of claim 20 or 21, wherein step a) and step b) are simultaneous.

23. The method of any of claims 18-22, further comprising incubating the resulting composition with nanotube seeds.

24. A method of measuring cellular shear stress, comprising:
obtaining a composition of any of claims 1-16; and
measuring azimuth angle between the nanotube and fluid flow direction with different fluid flow rates.

25. The method of claim 24, further comprising determining a mean total angle of nanotube rotation at each flow rate.

26. The method of claim 24, further comprising plotting the azimuth angle distribution for reach flow rate to determine angle probability versus angle.

27. A kit comprising:
a cell; and
a nucleic acid nanotube or nanotube seeds.

28. The kit of claim 27, further comprising at least one or all of
a primary antibody configured to bind a moiety located on the surface of the cell
a secondary antibody;
a biotinylated single-stranded polynucleotide; and
a plurality of nanotube monomers.

29. The kit of claim 28, wherein the secondary antibody is streptavidin conjugated.

30. The kit of claim 28, wherein the secondary antibody is biotinylated and the kit further comprises streptavidin or neutravidin.

31. The kit of any one of claims 27-30, wherein the biotinylated single-stranded polynucleotide is complementary to at least a portion of a single-stranded polynucleotide of the nanotube or nanotube seeds.
